(19) United States
(12) Patent Application Publication
(10) Pub. No.: US 2023/0309389 A1
(43) Pub. Date: Sep. 28, 2023

(54) CONDENSED CYCLIC COMPOUND, ORGANIC LIGHT EMITTING DEVICE INCLUDING CONDENSED CYCLIC COMPOUND, AND ELECTRONIC APPARATUS INCLUDING ORGANIC LIGHT EMITTING DEVICE

(71) Applicant: Samsung Electronics Co., Ltd., Suwon-si (KR)

(72) Inventors: Satoshi INAYAMA, Yokohama-city (JP); Eigo MIYAZAKI, Yokohama-city (JP); Keisuke KORAI, Yokohama-city (JP); Atsushi IMAMURA, Yokohama-city (JP)

(21) Appl. No.: 18/182,626

(22) Filed: Mar. 13, 2023

(30) Foreign Application Priority Data
Mar. 23, 2022 (JP) 2022-047282
Oct. 7, 2022 (KR) 10-2022-0129029

(51) Int. Cl. H10K 85/60; C07D 491/22; C07D 471/22; C07D 471/20; C09K 11/06

(52) U.S. Cl. CPC H10K 85/657; C07D 491/22; C07D 471/22; H10K 85/6572; C07D 471/20; C09K 11/06; H10K 50/11

(57) ABSTRACT

Provided are a condensed cyclic compound represented by Formula 1, an organic light-emitting device including the condensed cyclic compound, and an electronic apparatus including the organic light-emitting device:

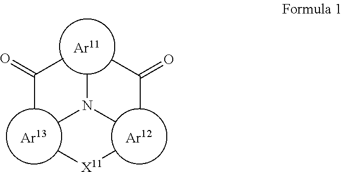

wherein, in Formula 1, the substituents may be understood by referring to the detailed description.

### CROSS-REFERENCE TO RELATED APPLICATIONS

This application claims priority under 35 U.S.C. § 119 to Japanese Patent Application No. 2022-047282, filed on Mar. 23, 2022, in the Japanese Patent Office and Korean Patent Application No. 10-2022-0129029, filed on Oct. 7, 2022, in the Korean Intellectual Property Office, the contents of which are incorporated herein in their entireties by reference.

### BACKGROUND

### 1. Field

The disclosure relates to a condensed cyclic compound, an organic light-emitting device including the condensed cyclic compound, and an electronic apparatus including the organic light-emitting device.

### 2. Description of the Related Art

Organic light-emitting devices (OLEDs) are self-emissive devices that, as compared with devices in the art, have wide viewing angles, high contrast ratios, short response times, and excellent brightness, driving voltage, and response speed characteristics, and produce full-color images.

OLEDs include an anode, a cathode, and an organic layer between the anode and the cathode and including an emission layer. A hole transport region may be between the anode and the emission layer, and an electron transport region may be between the emission layer and the cathode. Holes provided from the anode may move toward the emission layer through the hole transport region, and electrons provided from the cathode may move toward the emission layer through the electron transport region. The holes and the electrons recombine in the emission layer to produce excitons. The excitons may transition from an excited state to a ground state, thus generating light.

### SUMMARY

One or more embodiments relate to a condensed cyclic compound, an organic light-emitting device including the condensed cyclic compound, and an electronic apparatus including the organic light-emitting device, and more particularly to a condensed cyclic compound that may improve the luminescence efficiency of an organic light-emitting device, the condensed cyclic compound having a narrow half-width and improved colorimetric purity.

Additional aspects will be set forth in part in the description which follows and, in part, will be apparent from the description, or may be learned by practice of the presented embodiments of the disclosure.

According to one or more embodiments, a condensed cyclic compound is represented by Formula 1:

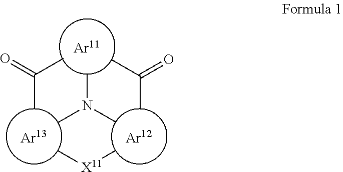

wherein, in Formula 1,
Ar¹¹, Ar¹², and Ar¹³ are each independently a substituted or unsubstituted group derived from an aromatic ring having 6 or more and 30 or less ring-forming atoms or a substituted or unsubstituted group derived from an aromatic ring having 5 or more and 30 or less ring-forming atoms,
X¹¹ is a single bond, —O—, —S—, —Se—, —NR^{X11}—, —CR^{X12}R^{X13}— or —SiR^{X14}R^{X15}—,
when X¹¹ is —O—, —S—, or —CR^{X12}R^{X13}—, Ar¹¹ is a substituted group derived from an aromatic ring having 6 or more and 30 or less ring-forming atoms or a substituted or unsubstituted group derived from an aromatic ring having 5 or more and 30 or less ring-forming atoms,
R^{X11}, R^{X12}, R^{X13}, R^{X14}, and R^{X15} are each independently a hydrogen atom, a deuterium atom, a substituted or unsubstituted alkyl group, a substituted or unsubstituted alkenyl group, a substituted or unsubstituted alkynyl group, a substituted or unsubstituted aryl group, a substituted or unsubstituted arylalkyl group, or a substituted or unsubstituted heteroaryl group,
R^{X12} and R^{X13} are optionally bound to each other to form a ring, and R^{X14} and R^{X15} are optionally bound to each other to form a ring.

According to an aspect of another embodiment, an organic light-emitting device may include a first electrode, a second electrode, and an organic layer between the first electrode and the second electrode and including an emission layer, and the organic light-emitting device may include the condensed cyclic compound.

According to an aspect of another embodiment, an electronic apparatus may include the organic light-emitting device.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a schematic cross-sectional view illustrating an organic light-emitting device according to an exemplary embodiment;

FIG. 2 is a schematic cross-sectional view illustrating an organic light-emitting device according to another exemplary embodiment;

FIG. 3 is a schematic cross-sectional view illustrating an organic light-emitting device according to still another exemplary embodiment;

FIG. 4A shows a 1H-NMR spectrum of Compound 1-4;

FIGS. 4B and 4C are each an enlarged view of the 1H-NMR spectrum in FIG. 4A, from 7 ppm to 10 ppm;

FIG. 5 is a view qualitatively illustrating an energy relationship;

FIG. 6 is a graph of rearrangement energy (eV) calculated by a FWHM-density functional method at photoluminescence (PL) measured in known condensed cyclic compounds R1 to R3; and

FIG. 7 shows electroluminescence (EL) spectra of organic EL devices of Examples 1 and 4 and Comparative Example 1.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

Unless otherwise defined, handling and measurement of physical properties may be performed at room temperature (from about 20° C. or higher and about 25° C. or lower) and at relative humidity (RH) from about 40% or greater and about 50% or less.

The term “X and Y may each independently be”, as used herein, may be understood that X and Y may be identical to or different from each other.

The term “group derived from a ring”, as used herein, refers to a group obtained by removing a hydrogen atom bound to a ring-forming atom in a ring structure.

The term “number of ring-forming atoms”, as used herein, refers to the number of atoms constituting the ring itself of a compound (e.g., a monocyclic compound, a condensed cyclic compound, a cross-linked compound, a carbocyclic compound, and a condensed cyclic compound) having a structure (e.g., a monocyclic ring, a condensed ring, and a ring assembly) in which atoms are bonded in a ring-like manner. The number of ring-forming atoms excludes the number of atoms that do not constitute the ring (e.g., a hydrogen atom that terminates a bond of atoms constituting the ring), and the number of atoms included in a substituent when the ring is substituted with the substituent. Unless otherwise specified, the same definition of the number of ring-forming atoms applies to descriptions provided below.

For example, a benzene ring has 6 ring-forming atoms, a naphthalene ring has 10 ring-forming atoms, a pyridine ring has 6 ring-forming atoms, and a furan ring has 5 ring-forming atoms.

When a benzene ring is substituted with, for example, an alkyl group as a substituent, the number of carbon atoms in the alkyl group is not included in the number of ring-forming atoms of the benzene ring. Accordingly, a benzene ring substituted with an alkyl group has 6 ring-forming atoms. In addition, when a naphthalene ring is substituted with, for example, an alkyl group as a substituent, the number of atoms in the alkyl group is not included in the number of ring-forming atoms of the naphthalene ring. Accordingly, a naphthalene ring substituted with an alkyl group has 10 ring-forming atoms. For example, the number of hydrogen atoms bound to a pyridine ring or the number of atoms constituting a substituent is not included in the number of ring-forming atoms of the pyridine ring. Accordingly, a pyridine ring to which a hydrogen atom or a substituent is bonded has 6 ring-forming atoms.

The term “substitution”, as used herein, refers to, unless otherwise defined, substitution with a deuterium atom, a halogen atom, a cyano group, an alkyl group, a haloalkyl group, an alkenyl group, an alkynyl group, —SiR¹¹R¹²R¹³ group, —NR¹⁴R¹⁵ group, an aryl group, an alkylaryl group, a heteroaryl group, an alkylheteroaryl group, an arylalkyl group, an alkoxy group, an aryloxy group, an alkylaryloxy group, a heteroaryloxy group, an alkylheteroaryloxy group, an alkylthio group, an arylthio group, an alkylarylthio group, a heteroarylthio group, or an alkylheteroarylthio group.

When two or more hydrogen atoms are substituted, the types of substituents may be the same or different.

A substituent is not the same as the group being substituted. For example, an alkyl group is not substituted with an alkyl group.

Examples of the halogen atom as a substituent may include a fluorine atom (F), a chlorine atom (Cl), a bromine atom (Br), and an iodine atom (I).

The alkyl group as a substituent may be linear, branched, or cyclic. A divalent alkyl group is referred to as an alkylene group. The number of carbon atoms in the alkyl group may be, but is not particularly limited to, 1 or more and 30 or less, or 1 or more and 20 or less. In addition, the number of carbons in the alkyl group may be 1 or more and 10 or less, or 1 or more and 6 or less. Examples of the alkyl group may include, but are not particularly limited to, a methyl group, an ethyl group, an n-propyl group, an isopropyl group, an n-butyl group, a sec-butyl group, a tert-butyl group, an isobutyl group, a 2-ethylbutyl group, a 3,3-dimethylbutyl group, an n-pentyl group, an isopentyl group, a neopentyl group, a tert-pentyl group (a t-pentyl group), a pentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, a cyclohexyl group, a 4-methylcyclohexyl group, a 4-tert-butylcyclohexyl group (a 4-t-butylcyclohexyl group), an n-heptyl group, a 1-methylheptyl group, a 2,2-dimethylheptyl group, a 2-ethylheptyl group, a 2-butylheptyl group, an n-octyl group, a tert-octyl group (a t-octyl group), a 2-ethyloctyl group, a 2-butyloctyl group, a 2-hexyloctyl group, a 3,7-dimethyloctyl group, a cyclooctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldecyl group, a 2-octyldecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-heneicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, and an n-triacontyl group.

As a substituent, the haloalkyl group is a group in which at least one hydrogen atom in the alkyl group is substituted with the halogen atom.

The alkenyl group as a substituent may be linear, branched, or cyclic. A divalent alkenyl group is referred to as an alkenylene group The number of carbon atoms in the alkenyl or alkenylene group may be, but is not particularly limited to, 2 or more and 30 or less, or 2 or more and 20 or less. In addition, the number of carbons in the alkenyl group may be 2 or more and 10 or less, or 2 or more and 6 or less. Examples of the alkenyl group may include, but are not particularly limited to, a vinyl group, a 2-prophenyl group, a 2-butenyl group, a 3-butenyl group, a 1-methyl-2-prophenyl group, a 2-methyl-2-prophenyl group, a 2-pentenyl group, a 3-pentenyl group, a 4-pentenyl group, a 1-methyl-2-butenyl group, a 2-methyl-2-butenyl group, a 1-methyl-3-butenyl group, a 2-methyl-3-butenyl group, a 3-methyl-3-butenyl group, a 1,1-dimethyl-2-prophenyl group, a 1,2-dimethyl-2-prophenyl group, and a 1-ethyl-2-prophenyl group.

The alkynyl group as a substituent may have linear, branched, or cyclic. A divalent alkynyl group is referred to as an alkynylene group The number of carbon atoms in the alkynyl or alkynylene group may be, but is not particularly limited to, 2 or more and 30 or less, or 2 or more and 20 or less. In addition, the number of carbons in the alkynyl group may be 2 or more and 10 or less, or 2 or more and 6 or less. Examples of the alkynyl group may include, but are not particularly limited to, a 2-butynyl group, a 3-pentyl group, a hexynyl group, a heptynyl group, an octynyl group, and decynyl group.

The aryl group as a substituent is not particularly limited, but may be, for example, a monovalent group derived from a hydrocarbon ring containing 1 or more aromatic rings. A divalent aryl group is referred to as an arylene group. In addition, the hydrocarbon ring constituting the aryl group may be a condensed ring. In addition, when the aryl group includes two or more aromatic rings, the two or more aromatic rings may be bound to each other via a single bond (in the form of a ring assembly of aromatic hydrocarbon rings).

Examples of the aryl group may include, but are not particularly limited to, a phenyl group, a naphthyl group, a phenanthryl group, biphenylenyl group, a triphenylene group, an anthryl group, a pyrenyl group, a fluorenyl group, an azulenyl group, an acenaphthyl group, a fluoranthenyl group, a naphthacenyl group, a perylenyl group, a pentacenyl group, a quaterphenyl group, and a chrysenyl group.

An alkylaryl group as a substituent is a group in which at least one hydrogen atom of the aryl group is substituted by an alkylene group, and the alkylene group and the aryl group may be the same as described above.

The number of carbons in the alkylaryl group may be, but is not particularly limited to, 7 or more and 30 or less, 7 or more 20 or less, or 7 or more and 10 or less.

Examples of the arylalkyl group include, but are not particularly limited to, a benzyl group, a phenethyl group, a diphenylmethyl group, and the like.

The heteroaryl group as a substituent may be, but is not particularly limited to, a monovalent group derived from a ring including one or more heteroaromatic rings having one or more heteroatoms (e.g., nitrogen atoms (N), oxygen atoms (O), phosphorus atoms (P), sulfur atoms (S), and silicon atoms (Si)) as ring-forming atoms, wherein the remaining ring-forming atoms are carbon atoms (C). A divalent heteroaryl group is referred to as an heteroarylene group. When the heteroaryl group includes two or more heteroatoms, the heteroatoms may be the same as or different from each other. In addition, a ring constituting the heteroaryl group may be a condensed ring. In addition, when the heteroaryl group includes two or more heteroaromatic rings, the two or more heteroaromatic rings may be bound to each other via a single bond. As such, the heteroaryl group may be a monocyclic heteroaryl group or a polycyclic heteroaryl group.

Examples of the heteroaryl group may include, but are not particularly limited to, a thienyl group, a furanyl group, a pyrrolyl group, an imidazole group, a thiazolyl group, an oxazolyl group, an oxadiazolyl group, a triazolyl group, a pyridyl group, a bipyridyl group, a pyrimidyl group, a triazinyl group, an acridinyl group, a pyridazinyl group, a pyridinyl group, a quinolinyl group, a quinazolinyl group, a quinoxalinyl group, a phenoxazinyl group, a phthalazinyl group, a pyridopyrimidinyl group, a pyridopyrazinyl group, a pyrazinopyrazinyl group, an isoquinolinyl group, an indolyl group a benzoxazolyl group, a benzimidazolyl group, a benzothiazolyl group, a benzothiophenyl group, a dibenzothienyl group, a thienothienyl group, a benzofuranyl group, a phenanthrolinyl group, an isoxazolyl group, an oxadiazolyl group, a thiazolyl group, a phenothiazolyl group, a phenothiazinyl group, a dibenzofuranyl group, and combinations thereof.

An alkylheteroaryl group as a substituent is a group in which at least one hydrogen atom of the heteroaryl group is substituted by an alkylene group, and the alkylene group and the heteroaryl group may be the same as described above.

The number of carbons in the arylalkyl group may be, but is not particularly limited to, 7 or more and 30 or less, 7 or more 20 or less, or 7 or more and 10 or less.

Examples of the alkylheteroaryl group include, but are not particularly limited to, a methylpyridine group and the like.

As a substituent, the alkoxy group has a structure of Formula: —O(A₁₀₀) group (wherein A₁₀₀ is an alkyl group), and the alkyl group is the same as described above.

As a substituent, the aryloxy group has a structure of Formula: —O(A₁₀₁) group (wherein A₁₀₁ is an aryl group), and the aryl group is the same as described above.

As a substituent, the alkylaryloxy group has a structure of Formula: —O(A₁₀₂) group (wherein A₁₀₂ is an alkylaryl group), and the alkylaryl group is the same as described above.

As a substituent, the heteroaryloxy group has a structure of Formula: —O(A₁₀₃) group (wherein A₁₀₃ is an heteroaryl group), and the heteroaryl group is the same as described above.

As a substituent, the alkylheteroaryloxy group has a structure of Formula: —O(A₁₀₄) group (wherein A₁₀₄ is an alkylheteroaryl group), and the alkylheteroaryl group is the same as described above.

As a substituent, the alkylthio group has a structure of Formula: —S(A₁₀₅) group (wherein A₁₀₅ is an alkyl group), and the alkyl group is the same as described above.

As a substituent, the arylthio group has a structure of Formula: —S(A₁₀₆) group (wherein A₁₀₆ is an aryl group), and the aryl group is the same as described above.

As a substituent, the alkylarylthio group has a structure of Formula: —S(A₁₀₇) group (wherein A₁₀₇ is an alkylaryl group), and the alkylaryl group is the same as described above.

As a substituent, the heteroarylthio group has a structure of Formula: —S(A₁₀₈) group (wherein A₁₀₈ is a heteroaryl group), and the heteroaryl group is the same as described above.

As a substituent, the alkylheteroarylthio group has a structure of Formula: —S(A₁₀₉) group (wherein A₁₀₉ is an alkylheteroaryl group), and the alkylheteroaryl group is the same as described above.

In the —SiR¹¹R¹²R¹³ group and the —NR¹⁴R¹⁵ group, R¹¹, R¹², R¹³, R¹⁴, and R¹⁵ may each independently be a hydrogen atom, a deuterium atom, an alkyl group, an alkenyl group, an alkynyl group, an aryl group, an arylalkyl group, or a heteroaryl group. The alkyl group, the alkenyl group, the alkynyl group, the aryl group, the arylalkyl group, and the heteroaryl group are each the same as described above.

Condensed Cyclic Compound

A condensed cyclic compound may be represented by Formula 1:

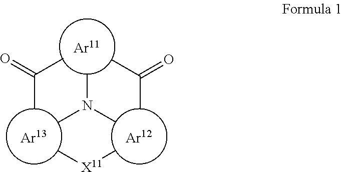

wherein, in Formula 1,
Ar¹¹, Ar¹², and Ar¹³ may each independently be a substituted or unsubstituted group derived from an aromatic ring having 6 or more and 30 or less ring-forming atoms or a substituted or unsubstituted group derived from an aromatic ring having 5 or more and 30 or less ring-forming atoms,
X¹¹ may be a single bond, —O—, —S—, —Se—, —NR^{X11}—, or —SiR^{X14}R^{X15}—,
when X¹¹ is —O—, —S—, or —CR^{X12}R^{X13}—, Ar¹¹ may be a substituted group derived from an aromatic ring having 6 or more and 30 or less ring-forming atoms or a substituted or unsubstituted group derived from an aromatic ring having 5 or more and 30 or less ring-forming atoms,
R^{X11}, R^{X12}, R^{X13}, R^{X14}, and R^{X15} may each independently be a hydrogen atom, a deuterium atom, a substituted or unsubstituted alkyl group, a substituted or unsubstituted alkenyl group, a substituted or unsubstituted alkynyl group, a substituted or unsubstituted aryl group, a substituted or unsubstituted arylalkyl group, or a substituted or unsubstituted heteroaryl group,
R^{X12} and R^{X13} may optionally be bound to each other to form a ring, and R^{X14} and R^{X15} may optionally be bound to each other to form a ring, and
the condensed cyclic compound may exclude the following compounds:

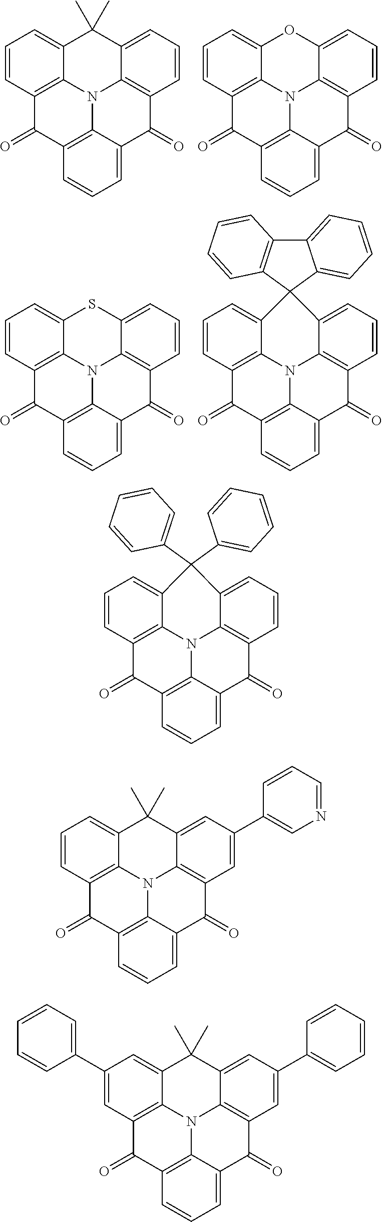

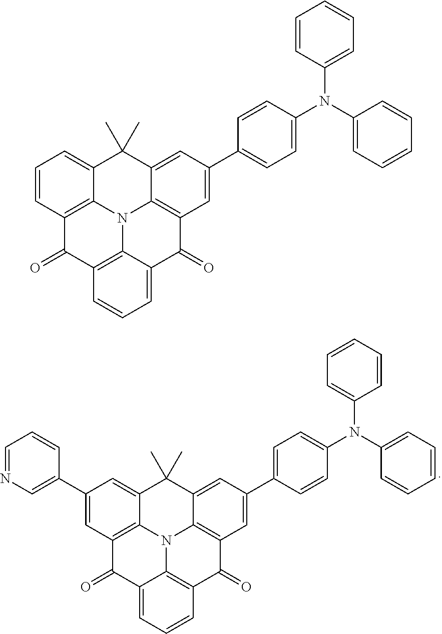 [SMILES: CC1(C)c2cccc3c(=O)c4cccc5c(=O)c6cc(-c7ccc(N(c8ccccc8)c8ccccc8)cc7)cc1c6n(c23)c45 ; CC1(C)c2cc(-c3ccc(N(c4ccccc4)c4ccccc4)cc3)cc3c(=O)c4cccc5c(=O)c6cc(-c7cccnc7)cc1c6n(c23)c45]

According to a first aspect, a condensed cyclic compound may be represented by Formula 1 and Formula 2:

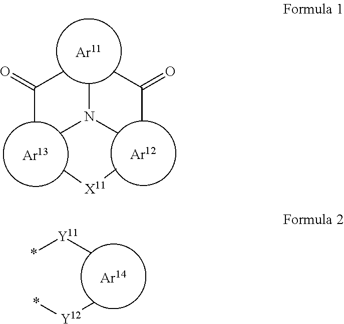

wherein, in Formulae 1 and 2,
Ar¹¹, Ar¹², Ar¹³, and Ar¹⁴ may each independently be a substituted or unsubstituted group derived from an aromatic ring having 6 or more and 30 or less ring-forming atoms or a substituted or unsubstituted group derived from an aromatic ring having 5 or more and 30 or less ring-forming atoms,
X¹¹ may be a single bond, —O—, —S—, —Se—, —NR^{X11}—, —CR^{X12}R^{X13}—, or —SiR^{X14}R^{X15}—,
R^{X11}, R^{X12}, R^{X13}, R^{X14}, and R^{X15} may each independently be a hydrogen atom, a deuterium atom, a substituted or unsubstituted alkyl group, a substituted or unsubstituted alkenyl group, a substituted or unsubstituted alkynyl group, a substituted or unsubstituted aryl group, a substituted or unsubstituted arylalkyl group, or a substituted or unsubstituted heteroaryl group,
Y¹¹ and Y¹² may each be a single bond, —O—, —S—, —Se—, —NR^{Y11}—, —CR^{Y12}R^{Y13}—, or —SiR^{Y14}R^{Y15}—,
R^{Y11}, R^{Y12}, R^{Y13}, R^{Y14}, and R^{Y15} may each independently be a hydrogen atom, a deuterium atom, a substituted or unsubstituted alkyl group, a substituted or unsubstituted alkenyl group, a substituted or unsubstituted alkynyl group, a substituted or unsubstituted aryl group, a substituted or unsubstituted arylalkyl group, or a substituted or unsubstituted heteroaryl group,
at least one of Y¹¹ and Y¹² may be a group other than a single bond, and
two * in Formula 2 may be bound to a ring-forming atom of Ar¹¹ in Formula 1, a ring-forming atom of Ar¹², or a ring-forming atom of Ar¹³.

In Formula 1, an aromatic ring constituting Ar¹¹, Ar¹², Ar¹³ and Ar¹⁴ may be a monocyclic ring or a condensed ring.

For example, the number of ring-forming atoms of the aromatic ring may be 6 or more and 30 or less, 6 or more and 20 or less, or 6 or more and 10 or less.

For example, the aromatic ring having 6 or more and 30 or less ring-forming atoms may be a benzene ring, a pentalene ring, an indene ring, a naphthalene ring, an anthracene ring, an azulene ring, a heptalene ring, an acenaphthalene ring, a phenalene ring, a fluorene ring, a phenanthrene ring, a biphenyl ring, a terpenyl ring, a triphenylene ring, a pyrene ring, a chrysene ring, a picene ring, a perylene ring, a pentaphene ring, a pentacene ring, a tetrapene ring, a hexapene ring, a hexacene ring, a rubicene ring, a trinaphthylene ring, a heptapene ring, or a pyranthrene ring.

In Formula 1, the heteroaromatic ring constituting Ar¹¹, Ar¹², Ar¹³, and Ar¹⁴ may be a ring including one or more heteroatoms (e.g., nitrogen atom (N), oxygen atom (O), phosphorus atom (P), sulfur atom (S), or silicon atom (Si)) as ring-forming atoms and carbon atoms (C) as remaining ring-forming atoms.

The heteroaromatic ring may be a monocyclic ring or a condensed ring.

For example, the number of ring-forming atoms of the heteroaromatic ring may be 5 or more and 30 or less, 5 or more and 20 or less, or 5 or more and 10 or less.

For example, the heteroaromatic ring having 5 or more and 30 or less ring-forming atoms may be a pyridine ring, a pyrazine ring, a pyridazine ring, a pyrimidine ring, a triazine ring, a quinoline ring, an isoquinoline ring, a quinoxaline ring, a quinazoline ring, a naphthyridine ring, an acridine ring, a phenazine ring, a benzoquinoline ring, a benzoisoquinoline ring, a phenanthridine ring, a phenanthroline ring, benzoquinone ring, a coumarin ring, an anthraquinone ring, a fluorenone ring, a furan ring, a thiophene ring, a benzofuran ring, a benzothiophene ring, a dibenzofuran ring, a dibenzothiophene ring, a pyrrole ring, an indole ring, a carbazole ring, an indolocarbazole ring, an imidazole ring, benzimidazole ring, a pyrazole ring, an indazole ring, an oxazole ring, an isooxazole ring, benzooxazole ring, a benzoisooxazole ring, a thiazole ring, an isothiazole ring, a benzothiazole ring, a benzoisothiazole ring, an imidazolinone ring, a benzimidazolinone ring, an imidazopyridine ring, an imidazopyrimidine ring, an imidazophenanthridine ring, an azadibenzofuran ring, an azacarbazole ring, an azadibenzothiophene ring, a diazadibenzofuran ring, a diazacarbazole ring, a diazadibenzothiophene ring, a xanthonering, or a thioxanthone ring.

In some embodiments, in Formula 1, X¹¹ may be a single bond or —CR^{X12}R^{X13}—.

In some embodiments, in Formula 1, when X¹¹ is —CR^{X12}R^{X13}—, R^{X12} and R^{X13} may each independently be a substituted or unsubstituted alkyl group or a substituted or unsubstituted aryl group.

For example, R^{X12} and R^{X13} may be the same group.

For example, R^{X12} and R^{X13} may each be a methyl group, an ethyl group, an n-propyl group, an iso-propyl group, an n-butyl group, a sec-butyl group, a tert-butyl group, an isobutyl group, or a phenyl group.

In some embodiments, one of Y¹¹ and Y¹² may be a single bond, and the other may be —O—, —S—, or —NR^{Y11}—.

In some embodiments, when one of Y¹¹ and Y¹² is —NR^{Y11}—, R^{Y11} may be an alkyl group or an aryl group.

In some embodiments, the group represented by Formula 2 may be present in the compound in the number of 1, 2, or 3. Examples of a binding form of the group represented by Formula 2 to the structure of Formula 1 may include the following forms.

1. Form including one group represented by Formula 2:
(1-1) Form where the group represented by Formula 2 is bound to a ring-forming atom of Ar¹¹ only; or
(1-2) Form where the group represented by Formula 2 is bound to a ring-forming atom of Ar¹³ only.

2. Form including two groups represented by Formula 2:
(2-1) Form where one group represented by Formula 2 is bound to a ring-forming atom of Ar¹¹, and another group represented by Formula 2 is bound to a ring-forming atom of Ar¹²; or
(2-2) Form where one group represented by Formula 2 is bound to a ring-forming atom of Ar¹¹, and another group represented by Formula 2 is bound to a ring-forming atom of Ar¹³; or
(2-3) Form where one group represented by Formula 2 is bound to a ring-forming atom of Ar¹², and another group represented by Formula 2 is bound to a ring-forming atom of Ar¹³.

3. Form including three groups represented by Formula 2:
(3-3) Form where one group represented by Formula 2 is bound to a ring-forming atom of Ar¹¹, another group represented by Formula 2 is bound to a ring-forming atom of A¹², and still another group represented by Formula 2 is bound to a ring-forming atom of A¹³.

The condensed cyclic compound according to the first aspect may be represented by Formula 3:

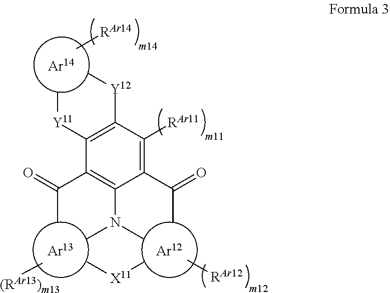

wherein, in Formula 3,
Ar¹², Ar¹³, Ar¹⁴, X¹¹, Y¹¹, and Y¹² may respectively be understood by referring to the descriptions of Ar¹², Ar¹³, Ar¹⁴, X¹¹, Y¹¹, and Y¹² in Formula 1 and 2,
R^{Ar11}, R^{Ar12}, R^{Ar13}, and R^{Ar14} may each independently be a deuterium atom, a halogen atom, a cyano group, an alkyl group, a haloalkyl group, an alkenyl group, an alkynyl group, —SiR¹¹R¹²R¹³, —NR¹⁴R¹⁵, an aryl group, an alkylaryl group, a heteroaryl group, an alkylheteroaryl group, an arylalkyl group, an alkoxy group, an aryloxy group, an alkylaryloxy group, a heteroaryloxy group, an alkylheteroaryloxy group, an alkylthio group, an arylthio group, an alkylarylthio group, a heteroarylthio group, or an alkylheteroarylthio group,
R¹¹, R¹², R¹³, R¹⁴, and R¹⁵ may each independently be a hydrogen atom, a deuterium atom, an alkyl group, an alkenyl group, an alkynyl group, an aryl group, an arylalkyl group, or a heteroaryl group,
m11 may be 0 or 1, and
m12, m13, and m14 may each independently be 0, 1, 2, 3, or 4.

In some embodiments, in Formula 3, m12, m13, and m14 may each independently be 0 or 1.

In some embodiments, the condensed cyclic compound according to the first aspect may be represented by one of Formulae 1-1 to 1-18:

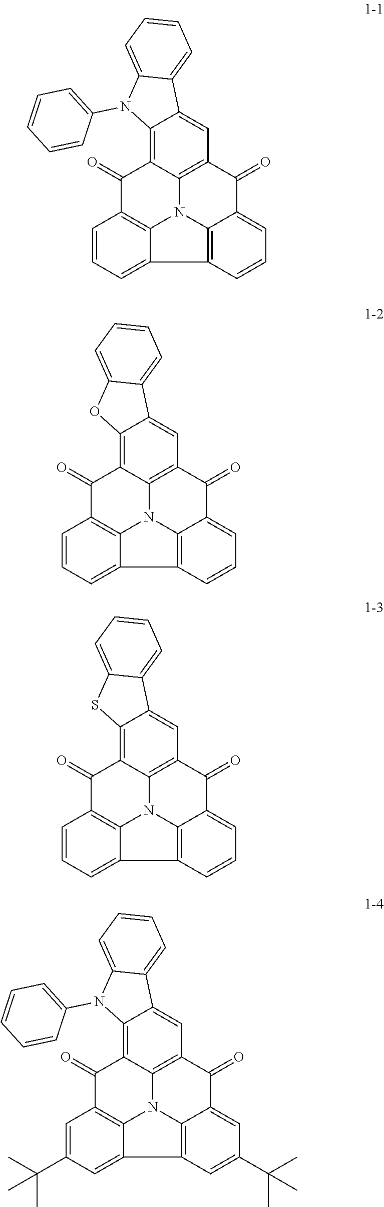

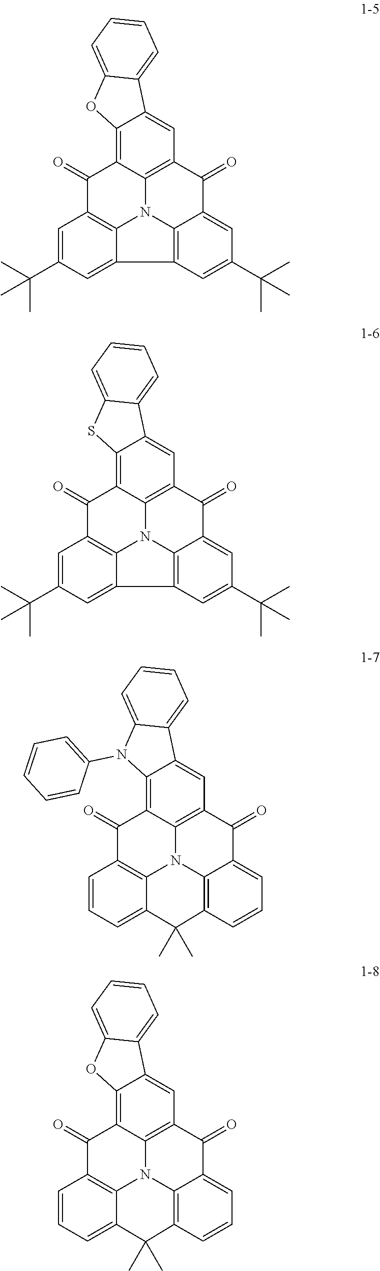

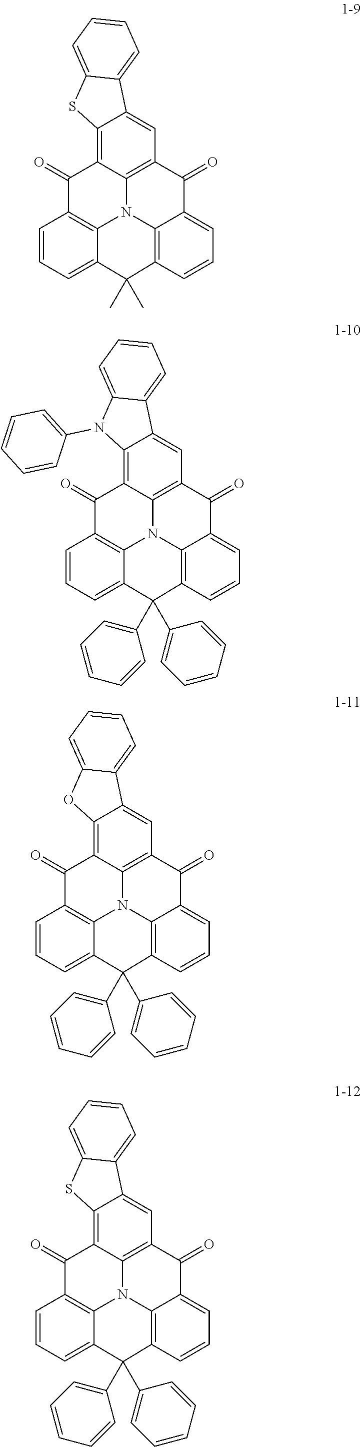

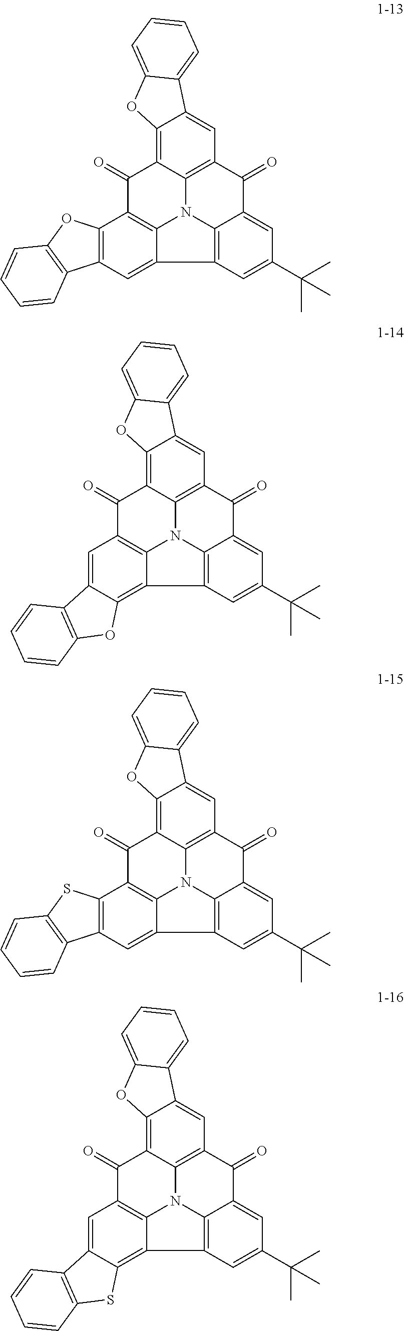 [SMILES: CC(C)(C)c1cc2c(=O)c3cc4c5ccccc5oc4c4c(=O)c5c6oc7ccccc7c6cc6c(c1)c2n(c34)c65 ; CC(C)(C)c1cc2c(=O)c3cc4c5ccccc5oc4c4c(=O)c5cc6c7ccccc7oc6c6c(c1)c2n(c34)c56 ; CC(C)(C)c1cc2c(=O)c3cc4c5ccccc5oc4c4c(=O)c5c6sc7ccccc7c6cc6c(c1)c2n(c34)c65 ; CC(C)(C)c1cc2c(=O)c3cc4c5ccccc5oc4c4c(=O)c5cc6c7ccccc7sc6c6c(c1)c2n(c34)c56]

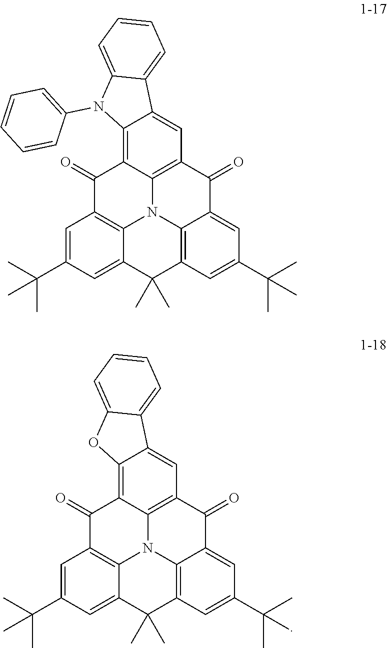

In some embodiments, the condensed cyclic compound according to the first aspect may be Compound 1-4, 1-5, 1-6, 1-17 or 1-18.

As shown in Formula 1, the condensed cyclic compound according to the first aspect may have a condensed ring structure in which Ar¹² and Ar¹³ are bonded by X¹¹. By having such a condensed ring structure, the molecular structure may be more robust (deformation of the molecular structure may be reduced), and high color purity light emission with a narrow spectrum width, in particular, high color purity blue light emission with a narrow spectrum width is realized.

In addition, the condensed cyclic compound according to the first aspect may have a structure where a ring-forming atom of Ar¹¹, a ring-forming atom of Ar¹², or a ring-forming atom of Ar¹³ in Formula 1 may be bound to Formula 2. By connecting these donor substituents, the value of highest occupied molecular orbital (HOMO) energy increases (HOMO level becomes shallow), and the difference in HOMO energy levels between the condensed cyclic compound and general host materials used in organic light-emitting devices may be reduced. As a result, an increase in driving voltage due to hole trap formation may be suppressed.

According to a second aspect, the condensed cyclic compound may be represented by Formula 4:

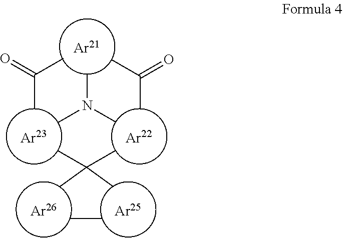

wherein, in Formula 4,
Ar²¹ may be (a) a substituted group derived from an aromatic ring having 6 or more and 30 or less ring-forming atoms or (b) a substituted or unsubstituted group derived from a heteroaromatic ring having 5 or more and 30 or less ring-forming atoms,
wherein (i) at least one hydrogen atom of (a) a group derived from an aromatic ring or (b) a group derived from a heteroaromatic ring may be substituted with an alkyl group, a haloalkyl group, —SiR²¹R²²R²³, —NR²⁴R²⁵, an alkylaryl group, an alkylheteroaryl group, an alkoxy group, an alkylaryloxy group, an alkylheteroaryloxy group, an alkylthio group, an alkylarylthio group, or an alkylheteroarylthio group (where R²¹, R²², R²³, R²⁴, and R²⁵ may each independently be a hydrogen atom, a deuterium atom, an alkyl group, an alkenyl group, an alkynyl group, an aryl group, an arylalkyl group, or a heteroaryl group) may be bound to two * in Formula 5, or (ii) a ring-forming atom of (a) a group derived from an aromatic ring or (b) a group derived from a heteroaromatic ring may be bound to two * in Formula 5,
Ar²², Ar²³, Ar²⁵, and Ar²⁶ may each independently be a substituted or unsubstituted group derived from an aromatic ring having 6 or more and 30 or less ring-forming atoms or a substituted or unsubstituted group derived from an aromatic ring having 5 or more and 30 or less ring-forming atoms,

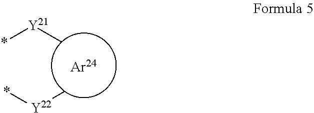

wherein, in Formula 5,
Ar²⁴ may be a substituted or unsubstituted group derived from an aromatic ring having 6 or more and 30 or less ring-forming atoms or a substituted or unsubstituted group derived from an aromatic ring having 5 or more and 30 or less ring-forming atoms,
Y²¹ and Y²² may be a single bond, —O—, —S—, —Se—, —NR^{Y21}—, —CR^{Y22}R^{Y23}—, or —SiR^{Y24}R^{Y25}—,
R^{Y21}, R^{Y22}, R^{Y23}, R^{Y24}, and R^{Y25} may each independently be a hydrogen atom, a deuterium atom, a substituted or unsubstituted alkyl group, a substituted or unsubstituted alkenyl group, a substituted or unsubstituted alkynyl group, a substituted or unsubstituted aryl group, a substituted or unsubstituted arylalkyl group, or a substituted or unsubstituted heteroaryl group, and
at least one of Y²¹ and Y²² may be a group other than a single bond.

In some embodiments, in Formula 5, one of Y₂₁ and Y₂₂ may be a single bond, and the other may be —O—, —S—, or —NR^{Y11}—.

In some embodiments, when one of Y²¹ and Y²² is —NR^{Y11}—, R^{Y21} may be an alkyl group, an aryl group, or an alkylaryl group.

The condensed cyclic compound according to the second aspect may be represented by Formula 6:

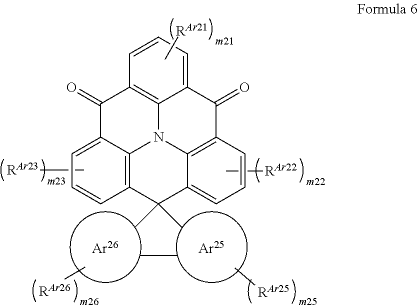

wherein, in Formula 6,
Ar²⁵ and Ar²⁶ may respectively be understood by referring to the descriptions of Ar²⁵ and Ar²⁶ in Formula 4,
R^{Ar21} may be an alkyl group, a haloalkyl group, —SiR²¹R²²R²³, —NR²⁴R²⁵, an alkylaryl group, an alkylheteroaryl group, an alkoxy group, an alkylaryloxy group, an alkylheteroaryloxy group, an alkylthio group, an alkylarylthio group, or an alkylheteroarylthio group,
R^{Ar22}, R^{Ar23}, R^{Ar25}, and R^{Ar26} may each independently be a deuterium atom, a halogen atom, a cyano group, an alkyl group, a haloalkyl group, an alkenyl group, an alkynyl group, —SiR²¹R²²R²³, —NR²⁴R²⁵, an aryl group, an alkylaryl group, a heteroaryl group, an alkylheteroaryl group, an arylalkyl group, an alkoxy group, an aryloxy group, an alkylaryloxy group, a heteroaryloxy group, an alkylheteroaryloxy group, an alkylthio group, an arylthio group, an alkylarylthio group, a heteroarylthio group, or an alkylheteroarylthio group,
R²¹, R²², R²³, R²⁴, and R²⁵ may each independently be a hydrogen atom, a deuterium atom, an alkyl group, an alkenyl group, an alkynyl group, an aryl group, an arylalkyl group, or a heteroaryl group,
m21 may be 1, 2, or 3,
m22 and m23 may each independently be 0, 1, 2, or 3, and
m25 and m26 may each independently be 0, 1, 2, 3, or 4.

In some embodiments, in Formula 6, m22, m23, m25, and m26 may each independently be 0 or 1.

The condensed cyclic compound according to the second aspect may be represented by Formula 7:

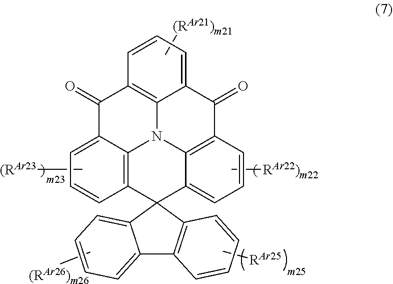

wherein, in Formula 7,
R^{Ar21}, R^{Ar22}, R^{Ar23}, R^{Ar25}, R^{Ar26}, m21, m22, m23, m25, and m26 may respectively be understood by referring to the descriptions of R^{Ar21}, R^{Ar22}, R^{Ar23}, R^{Ar25}, R^{Ar26}, m21, m22, m23, m25, and m26 in Formula 6.

In some embodiments, the condensed cyclic compound according to the second aspect may be represented by one of Formulae 2-1 to 2-29:

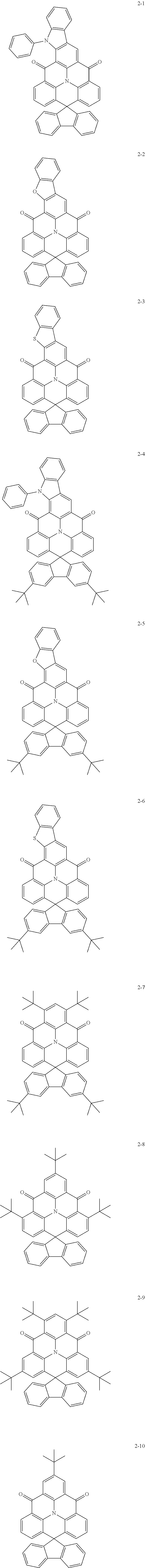

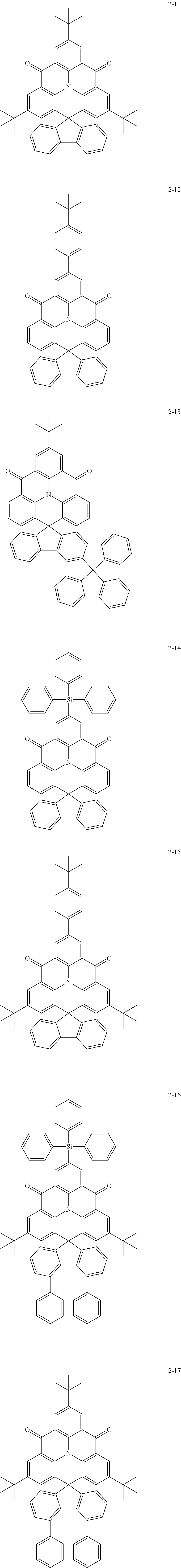

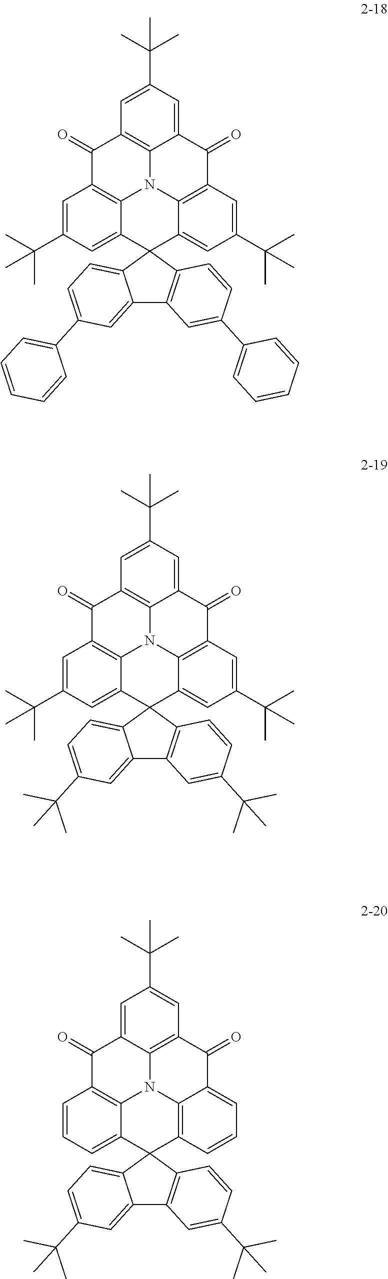

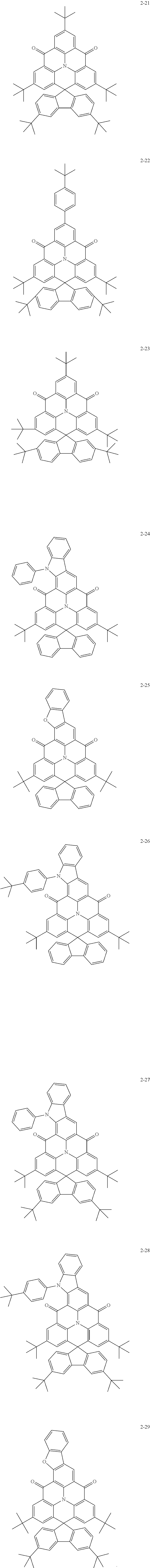

In some embodiments, the condensed cyclic compound according to the second aspect may be Compound 2-10, 2-11, 2-15, 2-19, 2-24, or 2-25.

The condensed cyclic compound according to the second aspect may have, as shown in Formula 4, i) a spiro ring structure including Ar²⁵ and Ar²⁶ and ii) Ar²¹ including a bulky substituent or the group represented by Formula 5 connected to Ar²¹.

A known Compound DiKTa has a large intermolecular interaction, and has characteristics of easy aggregation due to intermolecular interaction in a dispersed state in a high-concentration solution or thin film. Accordingly, the known Compound DiKTa has a problem in that high-purity blue light emission may not be obtained due to a long emission wavelength or a wide spectrum width due to molecular aggregation.

On the other hand, in the condensed cyclic compound according to the second aspect, by introducing a spiro ring structure orthogonal to the molecular plane and a bulky substituent or the group represented by Formula 5, intermolecular interactions may be reduced due to steric hindrance. As a result, a longer emission wavelength and spread of the spectral width due to molecular aggregation are suppressed, and high-purity blue emission is realized.

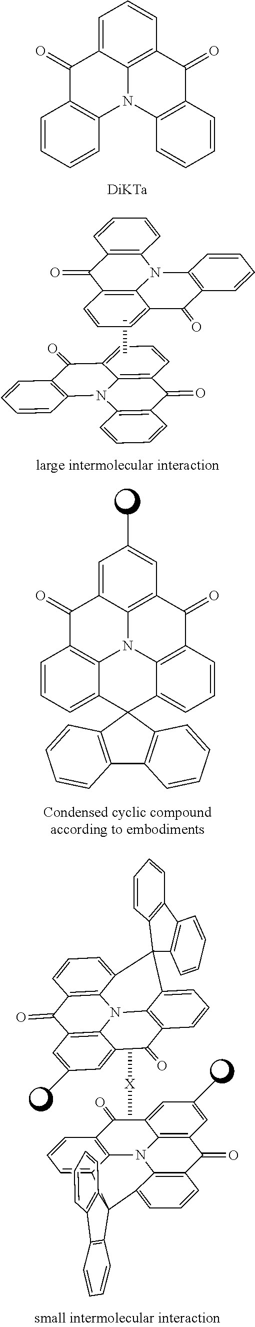

The condensed cyclic compound according to an embodiment may exhibit high colorimetric purity. Here, colorimetric purity may be indexed by rearrangement energy (g) or fluorescence spectral width (full width at half maximum of a fluorescence spectral peak, FWHM) in photoluminescence (PL). The smaller the rearrangement energy of the condensed cyclic compound and the narrower the FWHM, the higher the colorimetric purity.

In particular, the rearrangement energy of the condensed cyclic compound may be about 0 eV or more and about 0.110 eV or less, about 0 eV or more and about 0.100 eV or less, or about 0 eV or more and about 0.090 eV or less. In particular, the rearrangement energy of the condensed cyclic compound may be about 0 eV or more and about 0.080 eV or less or about 0 eV or more and about 0.075 eV or less. The FWHM of the condensed cyclic compound may be greater than about 0 nm and about 30 nm or less, or for example, greater than about 0 nm and about 25 nm or less, or greater than about 0 nm and about 20 nm or less. When the rearrangement energy and FWHM are within any of these ranges, emission of improved colorimetric purity may be realized.

The HOMO level of the condensed cyclic compound according to an embodiment may be, but is not particularly limited to, about −5.80 eV or more and about −4.40 eV or less, for example, about −5.60 eV or more and about −4.50 eV or less, or for example, about −5.40 eV or more and about −4.46 eV or less. When the HOMO level is within any of these ranges, the difference in HOMO energy with general host materials used in organic light-emitting devices may be small. Accordingly, the exiplex phenomenon is suppressed.

The LUMO level of the condensed cyclic compound according to an embodiment may be, but is not particularly limited to, about −2.40 eV or more and about −0.80 eV or less, for example, about −2.20 eV or more and about −1.00 eV or less, or for example, about −2.10 eV or more and about −1.16 eV or less. For example, the LUMO level of the condensed cyclic compound according to an embodiment may be about −2.00 eV or more and about −1.30 eV or less. When the LUMO level is within any of these ranges, the difference in LUMO energy with general host materials may be small. As a result, an increase in driving voltage due to electron trap formation may be further suppressed.

The peak wavelength in photoluminescence of the condensed cyclic compound may be, but is not particularly limited to, about 360 nm or more and about 515 nm or less, about 380 nm or more and about 505 nm or less, or for example, about 400 nm or more and about 500 nm or less. The peak wavelength in photoluminescence of the condensed cyclic compound may be, for example, about 420 nm or more and about 490 nm or less or about 425 nm or more and about 480 nm or less. When the peak wavelength is within any of these ranges, satisfactory emission, in particular, satisfactory blue emission may be obtained. The peak wavelength may be obtained by converting the adiabatic first excited singlet state (S₁) energy (hereinafter, also referred to as ‘adiabatic S₁ excitation energy’) level (eV) into an optical wavelength (nm).

The peak wavelength in photoluminescence (PL) and the FWHM in PL may each be measured and/or calculated using a spectrofluorophotometer F-7000 manufactured by Hitachi High-Tech Science Co., Ltd. The measurement and/or calculation methods are described in the Examples.

The HOMO, LUMO, peak wavelength, and ΔE_{ST} were calculated by using Gaussian 16 (Gaussian Inc.) according to a density functional theory (DFT). The detailed calculation method is the same as described in the Examples.

The synthesis method of the condensed cyclic compound according to one or more embodiments is not particularly limited and may be synthesized according to a known synthesis method. In particular, the condensed cyclic compound may be synthesized according to or in view of the method described in the Examples. For example, in the method described in the Examples, the condensed cyclic compound according to one or more embodiments may be synthesized through modifications such as changing raw materials and reaction conditions, adding or excluding some processes, or appropriately combining with other known synthesis methods.

The method of identifying a structure of the condensed cyclic compound according to one or more embodiments is not particularly limited. The condensed cyclic compound according to one or more embodiments may be identified by a known method, for example, NMR or LC-MS.

Material for Organic Light-Emitting Device

Another embodiment relates to a material for an organic light-emitting device including the condensed cyclic compound. The material for the organic light-emitting device may include the condensed cyclic compound and other materials used in an organic light-emitting device.

The other materials used in an organic light-emitting device may be, but are not limited to, materials known in the art. For example, as the other materials used in an organic light-emitting device, materials constituting each layer described in the below description of the organic light-emitting device may be used. Among the materials constituting each layer, a dopant material or a host material described in the below description of an emission layer of the organic light-emitting device may be used. In addition, a TADF material, a phosphorescent material, or a host material described in the below description of the emission layer of the organic light-emitting device may be used. In this regard, the phosphorescent material may be a platinum complex described herein.

Therefore, as an embodiment, a material for an organic light-emitting device that further includes a TADF material or a phosphorescent material described herein in addition to the condensed cyclic compound may be used. In particular, by including a TADF material or a phosphorescent material in addition to the condensed cyclic compound as an emission layer material, luminescence efficiency and/or lifespan of an organic light-emitting device may be remarkably improved.

The material for an organic light-emitting device may be a liquid material further including a solvent. The solvent may be, but is not particularly limited to, a solvent having a boiling point of about 100° C. or more and about 350° C. or less at atmospheric pressure (101.3 kPa, 1 atm). In an embodiment, the boiling point of the solvent at atmospheric pressure may be about 150° C. or more and about 320° C. or less, or about 180° C. or more and about 300° C. or less. When the above range is satisfied, the processability or film-forming capability of a wet film forming method may be improved, especially in an inkjet method.

The solvent having a boiling point of about 100° C. or more and about 350° C. or less at atmospheric pressure is not particularly limited, and any suitable solvent known in the art may be appropriately used. Hereinafter, the solvent having a boiling point of about 100° C. or more and about 350° C. or less at atmospheric pressure will be described in detail, but embodiments of the disclosure are not limited thereto.

Examples of a hydrocarbon-based solvent may include octane, nonane, decane, undecane, dodecane, and the like. Examples of an aromatic hydrocarbon-based solvent may include toluene, xylene, ethylbenzene, n-propyl benzene, iso-propyl benzene, mesitylene, n-butyl benzene, sec-butyl benzene, 1-phenyl pentane, 2-phenyl pentane, 3-phenyl pentane, phenyl cyclopentane, phenyl cyclohexane, 2-ethyl biphenyl, 3-ethyl biphenyl, and the like. Examples of an ether-based solvent may include 1,4-dioxane, 1,2-diethoxyethane, diethyleneglycol dimethyl ether, diethyleneglycol diethyl ether, anisole, ethoxybenzene, 3-methylanisole, m-dimethoxy benzene, and the like. Examples of a ketone-based solvent may include 2-hexanone, 3-hexanone, cyclohexanone, 2-heptanone, 3-heptanone, 4-heptanone, cycloheptanone, and the like. Examples of an ester-based solvent may include butyl acetate, butyl propionate, heptyl butyrate, propylene carbonate, methyl benzoate, ethyl benzoate, 1-propyl benzoate, 1-butyl benzoate, and the like. Examples of a nitrile-based solvent may include benzonitrile, 3-methyl benzonitrile, and the like. Examples of an amide-based solvent may include dimethyl formamide, dimethyl acetamide, N-methyl pyrrolidone, and the like. Such solvent may be used alone or in combination of two or more.

The material for an organic light-emitting device according to an embodiment may be a material for an emission layer.

The material for an organic light-emitting device according to an embodiment may not be a liquid composition. That is, the material for an organic light-emitting device may be substantially free of a solvent. In this regard, the term “material substantially free of a solvent” indicates that the amount of the solvent is less than 1 wt % based on the total weight of the composition. When the material for an organic light-emitting device is not a liquid composition, the organic light-emitting device may be substantially free of a solvent, and may not include a solvent (wherein the amount of the solvent is 0 wt % based on the total weight of the composition).

An amount of the condensed cyclic compound based on the total weight (in the case of a liquid composition, the total weight excluding the solvent) of the material for an organic light-emitting device (in particular, the material for an emission layer) is the same as an amount of the condensed cyclic compound based on the total weight of the emission layer of the organic light-emitting device described herein.

In addition, an amount of the TADF material or the phosphorescent material (specifically, the phosphorescent material) based on the total weight (in the case of a liquid composition, the total weight excluding the solvent) of the material for an organic light-emitting device (in particular, the material for an emission layer) is the same as an amount of the TADF material or the phosphorescent material (specifically, the phosphorescent material) based on the total weight of the emission layer of the organic light-emitting device described herein.

A content (parts by weight) of the TADF material or the phosphorescent material (specifically, the phosphorescent material) based on 100 parts by weight of the condensed cyclic compound in the material for an organic light-emitting device (in particular, the material for an emission layer) may also be the same.

In addition, an amount of the host material based on the total weight (in the case of a liquid composition, the total weight excluding the solvent) of the material for an organic light-emitting device (in particular, the material for an emission layer) is the same as an amount of the host material based on the total weight of the emission layer of the organic light-emitting device described herein.

A content (parts by weight) of the host material based on 100 parts by weight of the condensed cyclic compound in the material for an organic light-emitting device (in particular, the material for an emission layer) may also be the same as a content (parts by weight) of the host material based on 100 parts by weight of the condensed cyclic compound in the emission layer of the organic light-emitting device described herein.

When the amounts of the condensed cyclic compound, the TADF material or the phosphorescent material, and the host material in the material for an organic light-emitting device are within the above ranges, respectively, an organic light-emitting device having improved luminescence efficiency and/or lifespan may be obtained according to the emission color purity.

Organic Light-Emitting Device

Another embodiment relates to an organic light-emitting device including an organic layer including the condensed cyclic compound. The organic light emitting device may have a narrow emission spectrum, and may realize luminescence with high colorimetric purity. In addition, the organic light-emitting device may realize improved luminescence efficiency.

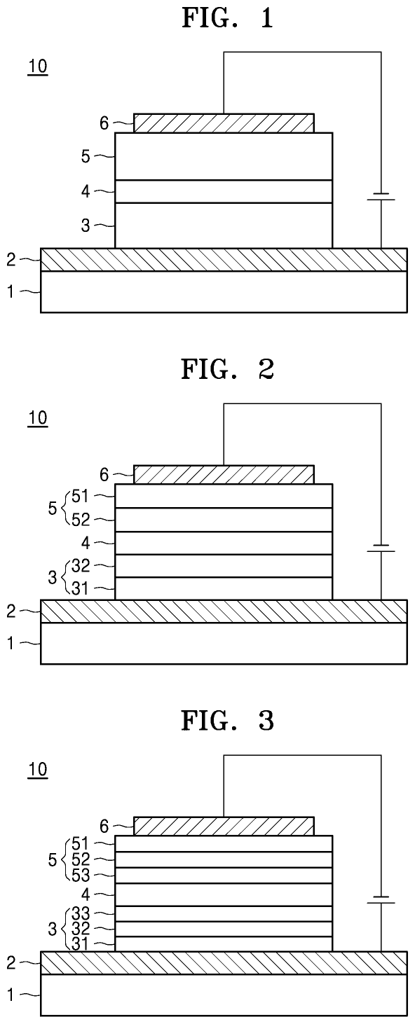

Descriptions of FIGS. 1 to 3

Hereinafter, with reference to FIGS. 1 to 3, an embodiment of an organic light-emitting device 10 will be described in detail.

FIG. 1 is a schematic view of an organic light-emitting device according to an embodiment. The organic light-emitting device 10 according to an embodiment may include a substrate 1, a first electrode 2, a hole transport region 3, an emission layer 4, an electron transport region 5, and a second electrode 6, which are sequentially layered in the stated order.

FIG. 2 is a schematic view of an organic light-emitting device according to another embodiment. The organic light-emitting device 10 according to an embodiment may include the substrate 1, the first electrode 2, the hole transport region 3, the emission layer 4, the electron transport region 5, and the second electrode 6. As shown in FIG. 2, the hole transport region 3 may include a hole injection layer 31 and a hole transport layer 32, which are sequentially layered in the stated order. In addition, as shown in FIG. 2, the electron transport region 5 may include an electron transport layer 52 and an electron injection layer 51, which are sequentially layered in the stated order.

FIG. 3 is a schematic view of an organic light-emitting device according to still another embodiment. The organic light-emitting device 10 according to an embodiment may include the substrate 1, the first electrode 2, the hole transport region 3, the emission layer 4, the electron transport region 5, and the second electrode 6. As shown in FIG. 3, the hole transport region 3 may include the hole injection layer 31, the hole transport layer 32, and an electron blocking layer 33, which are sequentially layered in the stated order. In addition, as shown in FIG. 2, the electron transport region 5 may include a hole blocking layer 53, the electron transport layer 52, and the electron injection layer 51, which are sequentially layered in the stated order.

An embodiment may include, for example, an organic electroluminescence device including a first electrode, a second electrode, and a single or a plurality of emission layers. The second electrode may be one the first electrode.

In the present specification, “on” may not apply only to a case of “just on” another part and may also include a case where another part may be present therebetween. Similarly, when a part such as a layer, a membrane, a regions, a plate, or the like is described as being “below” or “under” another part, a case of “just under” another part and also a case where another part present therebetween may be included.

In the present specification, “arrangement” may include a case where a portion is arranged not only on an upper part but also on a lower part.

The organic light-emitting device 10 may include the condensed cyclic compound according to one or more embodiments. For example, the condensed cyclic compound according to one or more embodiments may include an organic layer between the first electrode 2 and the second electrode 6. In some embodiments, the condensed cyclic compound may include the emission layer 4.

An embodiment in which the emission layer includes the condensed cyclic compound according to one or more embodiments will be described below.

Emission Layer 4

The emission layer 4 may emit light by fluorescence or phosphorescence.

The emission layer 4 may be a single layer including a single material or a single layer including a plurality of different materials. In addition, the emission layer 4 may have a multilayer structure having multiple layers including a plurality of different materials.

In the emission layer 4, the condensed cyclic compound may be used alone or two or more thereof may be combined.

The content of the condensed cyclic compound based on the total weight of the emission layer may be, but is not particularly limited to, 0.05 wt % or more. In particular, the content of the condensed cyclic compound based on the total weight of the emission layer may be 0.1 wt % or more, 0.2 wt % or more, 50 wt % or less, 30 wt %, or less, or 25 wt % or less. Within these ranges, an organic light-emitting device having improved colorimetric purity, luminescence efficiency and/or lifespan may be obtained.

In some embodiments, the emission layer 4 may further include a host, wherein the host and the condensed cyclic compound may be different from each other. The host may not emit light, and the condensed cyclic compound may emit light. That is, the condensed cyclic compound may be a dopant.

In some embodiments, the emission layer 4 may further include a host and a dopant, wherein the host, the dopant, and the condensed cyclic compound may be different from one another. In this embodiment, the host and the condensed cyclic compound may not each emit light, and the dopant may emit light.

In the Examples, the host and the dopant will be described in more detail.

The emission layer 4 may include a known host material and a known dopant material.

For example, the emission layer may include, in addition to the condensed cyclic compound, an anthracene derivative, a pyrene derivative, a fluoranthene derivative, a chrysene derivative, a dihydrobenzoanthracene derivative, or a triphenylene derivative.

For example, the emission layer may include, as the host material, at least one of bis[2-(diphenylphosphino)phenyl]etheroxide (DPEPO), 4,4′-bis(carbazol-9-yl)biphenyl (CBP), 3,3′-bis(carbazol-9-yl)biphenyl (mCBP), 1,3-bis(carbazol-9-yl)benzene (mCP), 2,8-bis(diphenylphosphoryl)dibenzo[b,d]furan (PPF), 4,4′,4″-tris(carbazol-9-yl)triphenylamine (TcTa), and 1,3,5-tris(N-phenylbenzimidazole-2-yl)benzene (TPBi). However, embodiments are not limited thereto. The emission layer may include, for example, tris(8-hydroxyquinolino)aluminum (Alq3), 4,4′-bis(N-carbazolcarbazolyl)-1,1′-biphenyl (CBP), poly(n-vinylcarbazole (PVK), 9,10-di(naphthalen-2-yl)anthracene (ADN), 4,4′,4″-tris(carbazol-9-yl)-triphenylamine (TCTA), 1,3,5-tris(N-phenylbenzimidazol-2-yl) benzene (TPBi), 3-tert-butyl-9,10-di(naphth-2-yl)anthracene (TBADN), distyryl arylene (DSA), 4,4′-bis(9-carbazolyl)-2,2′-dimethyl-biphenyl (CDBP), 2-methyl-9,10-bis(naphthalen-2-yl)anthracene (MADN), bis[2-(diphenylphosphino)phenyl]etheroxide (DPEPO), hexaphenylcyclotriphosphazene (CP1), 1,4-bis(triphenylsilyl)benzene (IGH2), hexaphenylcyclotrisiloxane (DPSiO₃), octaphenylcyclotetrasiloxane (DPSiO₄), or 2,8-bis(diphenylphosphoryl) dibenzofuran (PPF).

In addition, the emission layer may include a material having a HOMO of −5.2 eV or less as a host material. In addition, the emission layer may include a material having a LUMO of −1.4 eV or less as a host material. By using a host material with low HOMO and LUMO and high electron transportability, an organic light-emitting device, especially a blue organic light-emitting device, may improve driving durability. Examples thereof include, but are not particularly limited to, Compound A represented by the following Formula disclosed in “An Alternative Host Material for Long-Lifespan Blue Organic Light-Emitting Diodes Using Thermally Activated Delayed Fluorescence” Soo-Ghang Ihn, Namheon Lee, Soon Ok Jeon, Myungsun Sim, Hosuk Kang, Yongsik Jung, Dal Ho Huh, Young Mok Son, Sae Youn Lee, Masaki Numata, Hiroshi Miyazaki, Rafael Gomez-Bombarelli, Jorge Aguilera-Iparraguirre, Timothy Hirzel, Alan Aspuru-Guzik, Sunghan Kim, and Sangyoon Lee, Advanced Science News 2017, 4, 1600502. When an emission layer is formed in combination with such a host material, in a blue light-emitting material in the related art, a deep hole trap may occur, which may cause undesirable effects such as an increase in driving voltage. On the other hand, as the condensed cyclic compound has weak hole trapping properties, it is expected to suppress an increase in driving voltage.

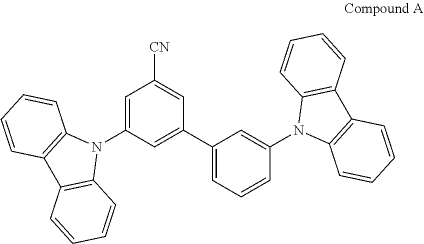 [SMILES: N#Cc1cc(-c2cccc(-n3c4ccccc4c4ccccc43)c2)cc(-n2c3ccccc3c3ccccc32)c1]

In addition, the emission layer may include the following compounds as a host material:

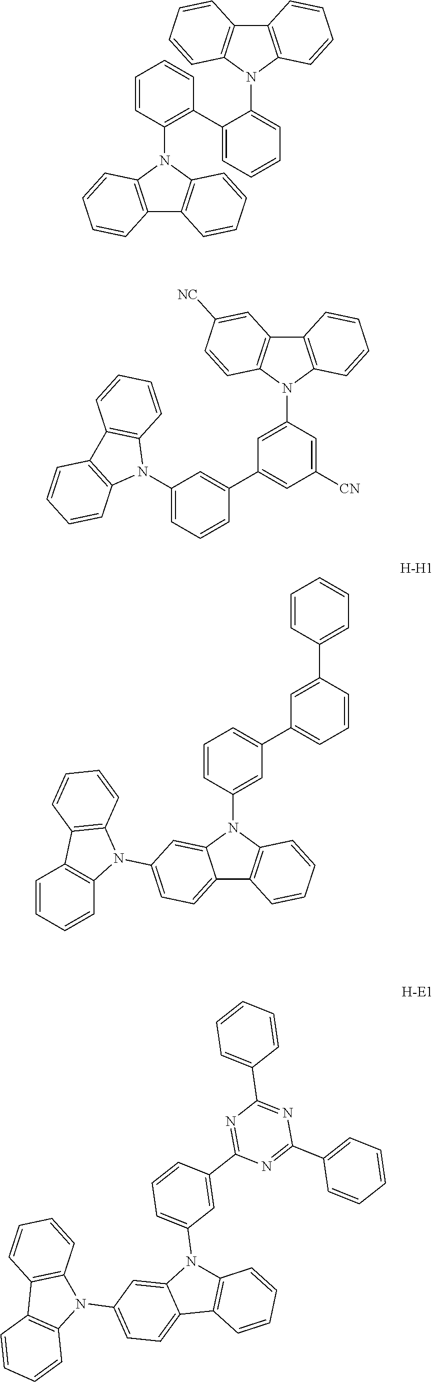 [SMILES: c1ccc(-n2c3ccccc3c3ccccc32)c(-c2ccccc2-n2c3ccccc3c3ccccc32)c1 ; N#Cc1cc(-c2cccc(-n3c4ccccc4c4ccccc43)c2)cc(-n2c3ccccc3c3cc(C#N)ccc32)c1 ; c1ccc(-c2cccc(-c3cccc(-n4c5ccccc5c5ccc(-n6c7ccccc7c7ccccc76)cc54)c3)c2)cc1 ; c1ccc(-c2nc(-c3ccccc3)nc(-c3cccc(-n4c5ccccc5c5ccc(-n6c7ccccc7c7ccccc76)cc54)c3)n2)cc1]

Among these compounds, the emission layer may include Compound H-H1 and/or Compound H-E1 as a host material, and in particular, a combination of Compound H-H1 and Compound H-E1.

The content of the host material based on the total weight of the emission layer may be, but is not particularly limited to 5 wt % or more. In some embodiments, the content of the host material may be 10 wt % or more or 20 wt % or more. In addition, the content of the host material based on the total weight of the emission layer may be 99 wt % or less. In some embodiments, the content may be 95 wt % or less or 90 wt % or less. Within these ranges, an organic light-emitting device having improved luminescence colorimetric purity, luminescence efficiency, and/or lifespan may be obtained.

When the emission layer includes a host material, the content thereof may be, but is not particularly limited to 1,000 parts by weight or more or 200,000 parts by weight or less based on 100 parts by weight of the condensed cyclic compound. In some embodiments, the content may be 2,000 parts by weight or more, 3,000 parts by weight or more, 150,000 parts by weight or less, or 100,000 parts by weight or less based on 100 parts by weight of the condensed cyclic compound. Within these ranges, an organic light-emitting device having improved luminescence colorimetric purity, luminescence efficiency, and/or lifespan may be obtained.

The emission layer is not particularly limited, but may contain, for example, a known dopant material. For example, the emission layer may include a styryl derivative (for example, 1,4-bis[2-(3-N-ethylcarbazolyl)vinyl]benzene (BCzVB), 4-(di-p-trilamino)-4′-[(di-p)-trilamino)styryl]stylbene (DPAVB), or N-(4-((E)-2-(6-((E)-4-(diphenylamino)styryl)naphthalen-2-yl)vinyl)phenyl)-N-phenylbenzeneamine (N-BDAVBi)), perylene or a derivative thereof (for example, 2,5,8,11-tetra-tert-butylperylene (TBP)), or pyrene or a derivative thereof (for example, 1,1-dipyrene, 1,4-dipyrenylbenzene, or 1,4-bis(N, N-diphenylamino)pyrene).

In addition, the emission layer may further include a thermally activated delayed fluorescent material (TADF Compound) or a phosphorescent material other than the condensed cyclic compound. The term “thermally activated delayed fluorescence” refers to a phenomenon in which reverse intersystem crossing occurs between triplet excitons and singlet excitons in a compound with a small energy difference (ΔE_{ST}) between the singlet level and the triplet level, and the term “TADF material” refers to a material in which such a phenomenon occurs.

As is known in the related art, in the emission layer of an organic light-emitting device, singlet excitons and triplet excitons are generated at a ratio of 1:3 by recombination of holes and electrons. In a device including only a fluorescent material as a luminescent material, only singlet excitons are involved in light emission, whereas in a device including a TADF material or a phosphorescent material as a luminescent material, both singlet excitons and triplet excitons may be used for light emission. Accordingly, the luminescence efficiency of the device including the TADF material or the phosphorescent material as a luminescent material may be significantly improved. Excitons generated on the TADF material or the phosphorescent material generally have a long lifespan of 1 μs or more. The excitons are in an unstable state with high energy, and thus, material degradation may occur while the excitons are present, leading to a reduction in device lifespan. When a TADF material or a phosphorescent material is present in the emission layer, in addition to the condensed cyclic compound, excitons are generated with high efficiency on the TADF material or the phosphorescent material, and energy may be transferred to the condensed cyclic compound through a Förster resonance energy transfer (FRET) mechanism. As a result, high-efficiency fluorescence may be obtained from the condensed cyclic compound, and the time for excitons to exist on the TADF material or the phosphorescent material may be shortened. Thus, the possibility of material deterioration may be significantly reduced, and the device lifespan may be significantly improved.

The amount of the TADF material or the phosphorescent material (in particular, the phosphorescent material) based on the total weight of the emission layer may be, but is not particularly limited to 0.1 wt % or more. In an embodiment, the amount may be 0.5 wt % or more, 1 wt % or more, 3 wt % or more, or 5 wt % or more. In addition, the amount of the TADF material or the phosphorescent material (in particular, the phosphorescent material) based on the total weight of the emission layer may be 50 wt % or less. In an embodiment, the amount may be 40 wt % or less, or 30 wt % or less. In addition, when the emission layer includes both the TADF material and the phosphorescent material, the total amount thereof may be within the above ranges. Within these ranges, an organic light-emitting device having improved luminescence colorimetric purity, luminescence efficiency, and/or lifespan may be obtained.

When the emission layer includes a TADF material or a phosphorescent material (e.g., a phosphorescent material), the content thereof may be, but is not particularly limited to, 100 parts by weight or more based on 100 parts by weight of the condensed cyclic compound. In some embodiments, the content may be 100 parts by weight or more or 200 parts by weight or more, based on 100 parts by weight of the condensed cyclic compound. The content of a TADF material or a phosphorescent material (e.g., a phosphorescent material) may be 1,000 parts by weight or less based on 100 parts by weight of the condensed cyclic compound. In some embodiments, the content may be 7,500 parts by weight or less or 5,000 parts by weight or less, based on 100 parts by weight of the condensed cyclic compound. In addition, when the emission layer includes both the TADF material and the phosphorescent material, the total amount thereof may be within the above ranges. Within these ranges, an organic light-emitting device having improved luminescence colorimetric purity, luminescence efficiency, and/or lifespan may be obtained.

Examples of the TADF material may include the following compounds:

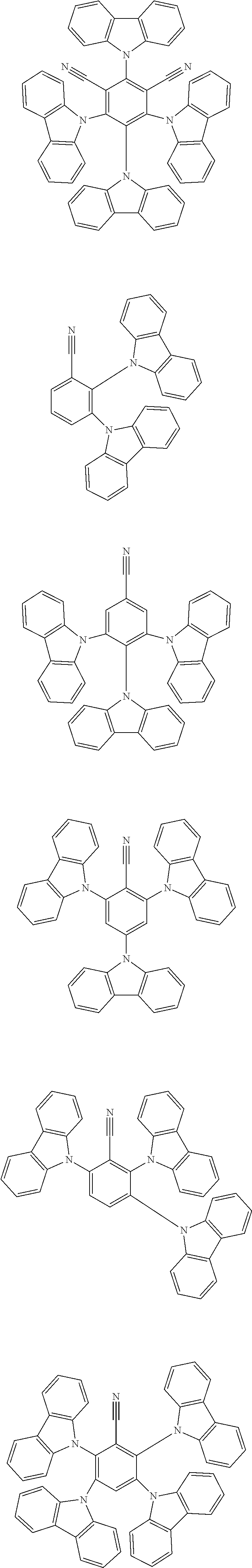 [SMILES: N#Cc1c(-n2c3ccccc3c3ccccc32)c(C#N)c(-n2c3ccccc3c3ccccc32)c(-n2c3ccccc3c3ccccc32)c1-n1c2ccccc2c2ccccc21 ; N#Cc1cccc(-n2c3ccccc3c3ccccc32)c1-n1c2ccccc2c2ccccc21 ; N#Cc1cc(-n2c3ccccc3c3ccccc32)c(-n2c3ccccc3c3ccccc32)c(-n2c3ccccc3c3ccccc32)c1 ; N#Cc1c(-n2c3ccccc3c3ccccc32)cc(-n2c3ccccc3c3ccccc32)cc1-n1c2ccccc2c2ccccc21 ; N#Cc1c(-n2c3ccccc3c3ccccc32)ccc(-n2c3ccccc3c3ccccc32)c1-n1c2ccccc2c2ccccc21 ; N#Cc1c(-n2c3ccccc3c3ccccc32)c(-n2c3ccccc3c3ccccc32)cc(-n2c3ccccc3c3ccccc32)c1-n1c2ccccc2c2ccccc21]

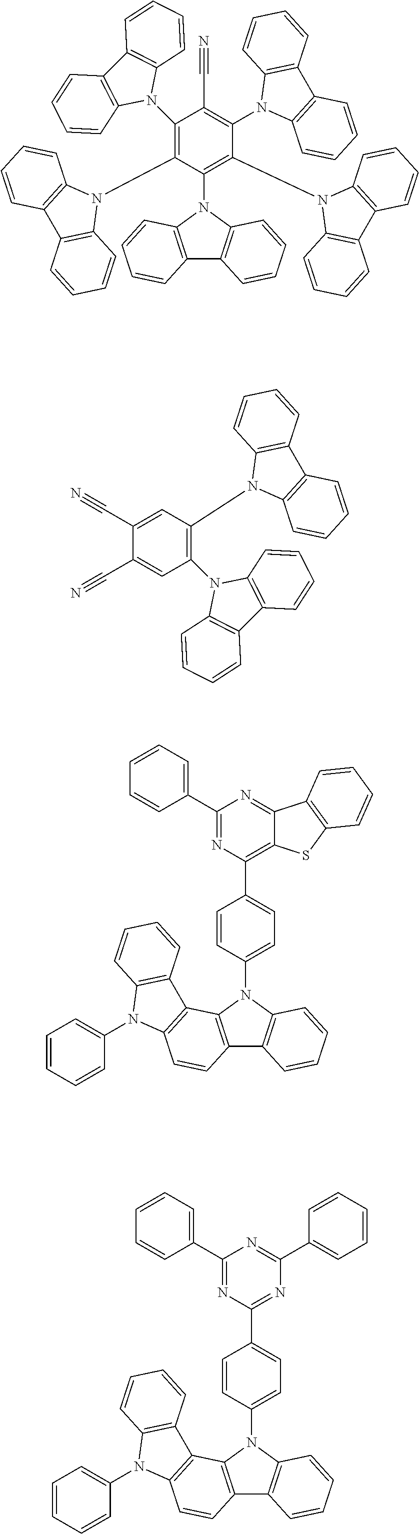 [SMILES: N#Cc1c(-n2c3ccccc3c3ccccc32)c(-n2c3ccccc3c3ccccc32)c(-n2c3ccccc3c3ccccc32)c(-n2c3ccccc3c3ccccc32)c1-n1c2ccccc2c2ccccc21 ; N#Cc1cc(-n2c3ccccc3c3ccccc32)c(-n2c3ccccc3c3ccccc32)cc1C#N ; c1ccc(-c2nc(-c3ccc(-n4c5ccccc5c5ccc6c(c7ccccc7n6-c6ccccc6)c54)cc3)c3sc4ccccc4c3n2)cc1 ; c1ccc(-c2nc(-c3ccccc3)nc(-c3ccc(-n4c5ccccc5c5ccc6c(c7ccccc7n6-c6ccccc6)c54)cc3)n2)cc1]

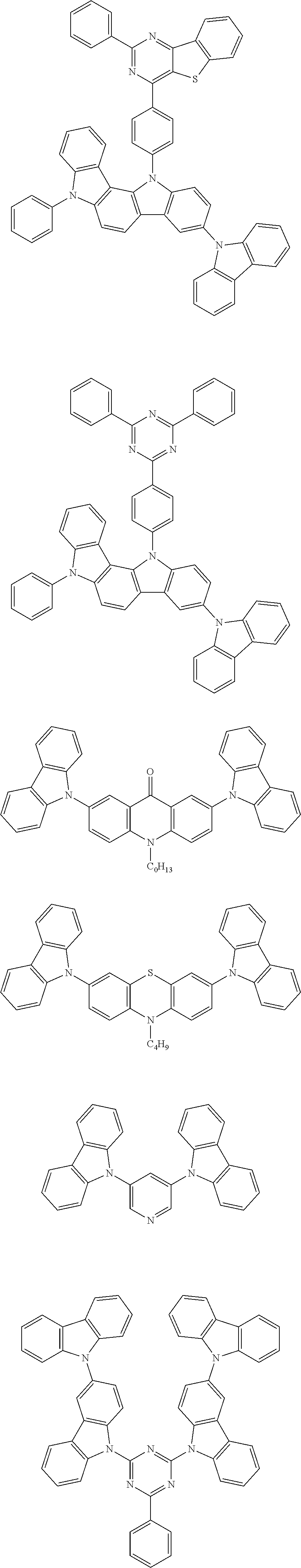

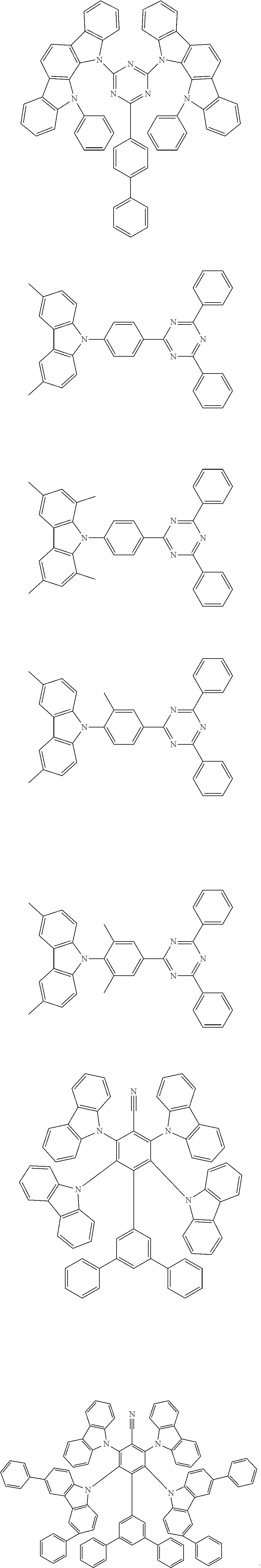

The TADF material may be used alone or in combination of two or more TADF materials.

In addition, the emission layer may include a phosphorescent material (phosphorescent compound) in addition to the condensed cyclic compound. The phosphorescent material (phosphorescent compound) is not particularly limited, and a known phosphorescent compound may be used. Among known phosphorescent compounds, a phosphorescent complex may be used, and in particular, a platinum complex may be used.

Examples of the phosphorescent material (phosphorescent compound) may include the following compounds:

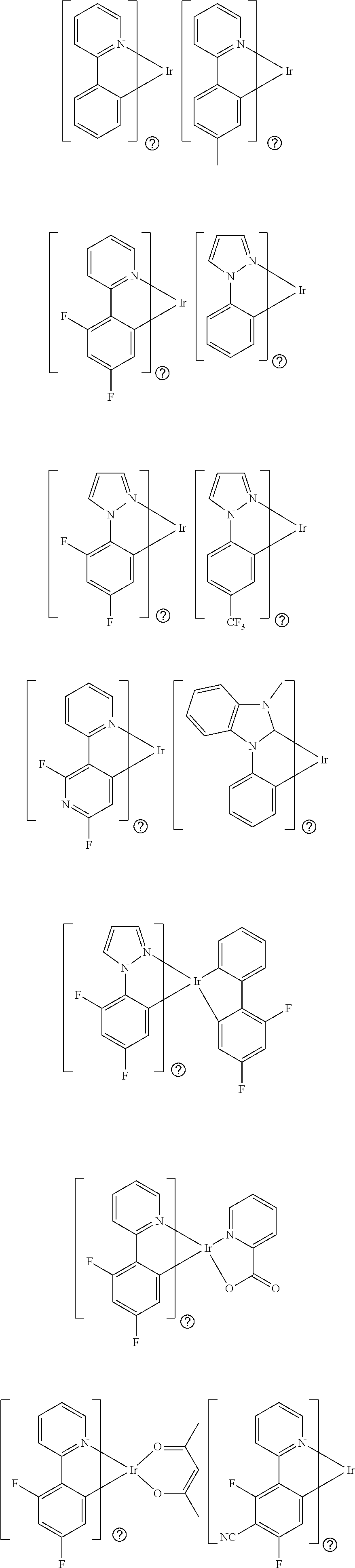

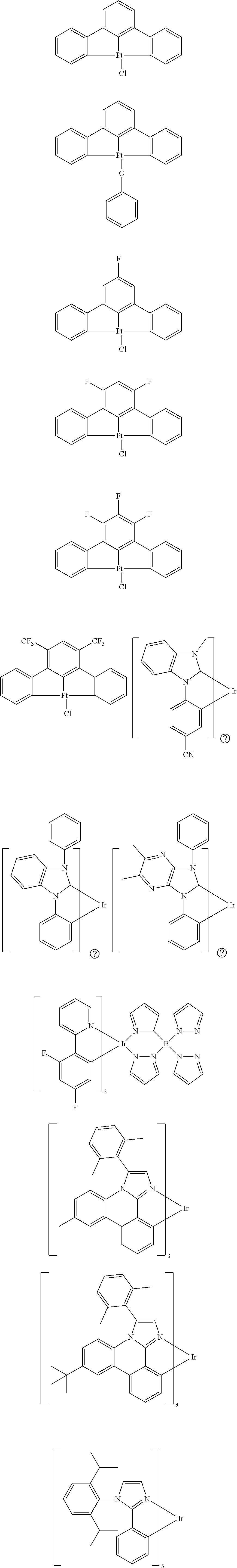

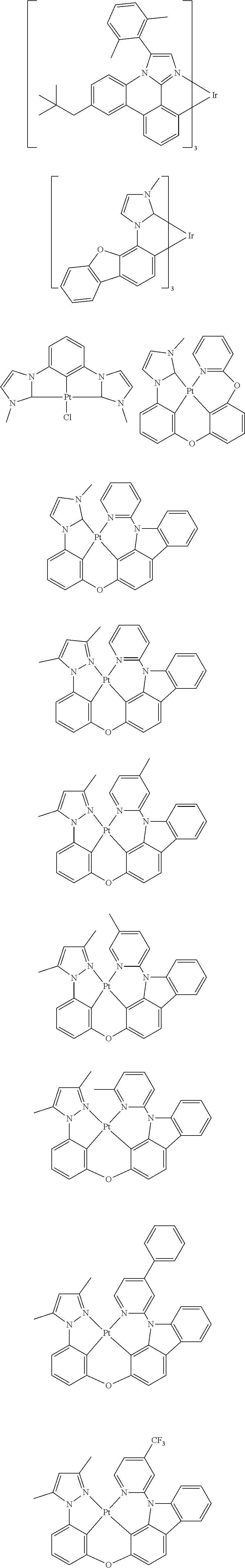

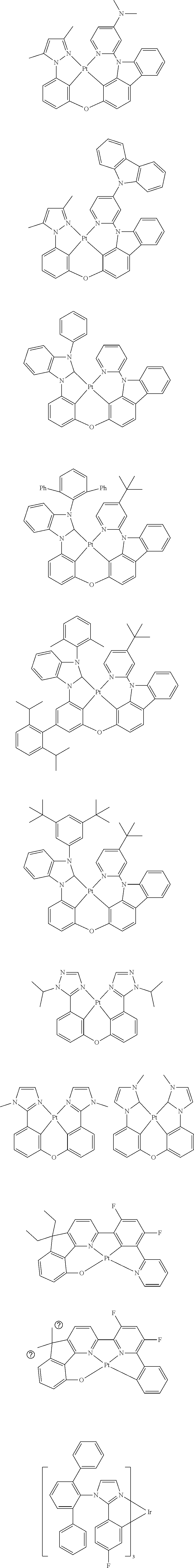

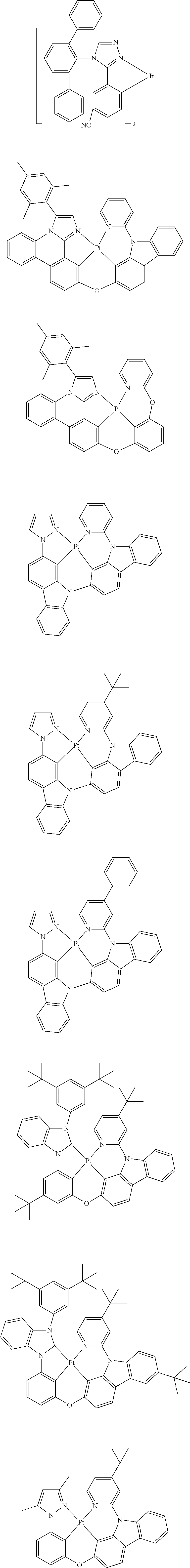

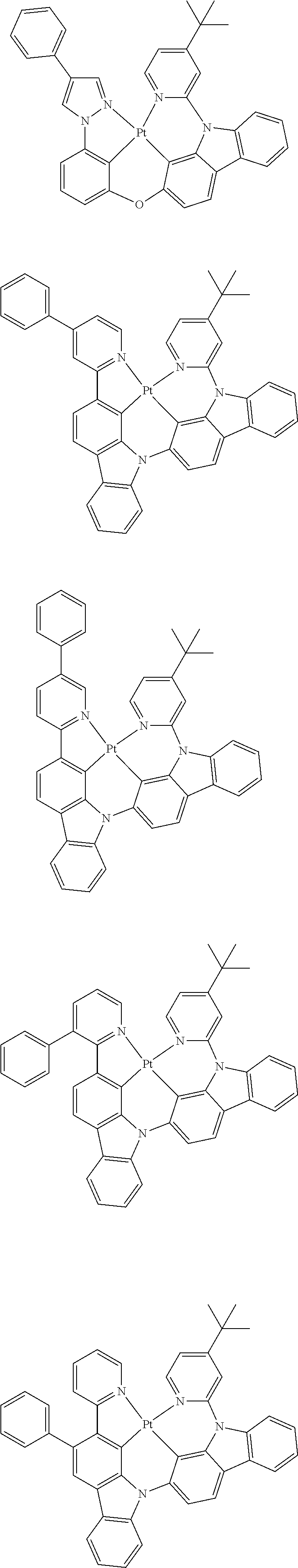

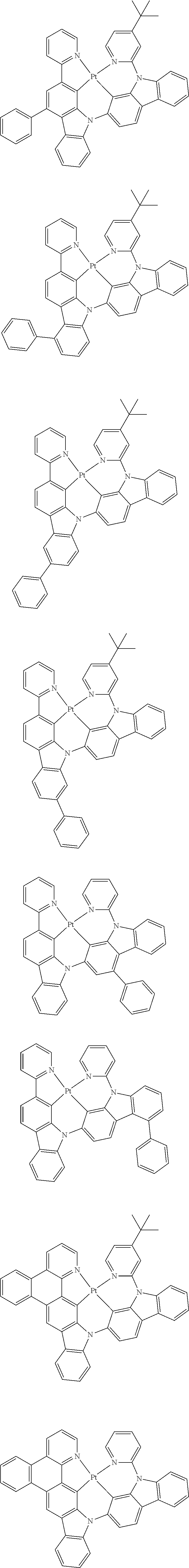

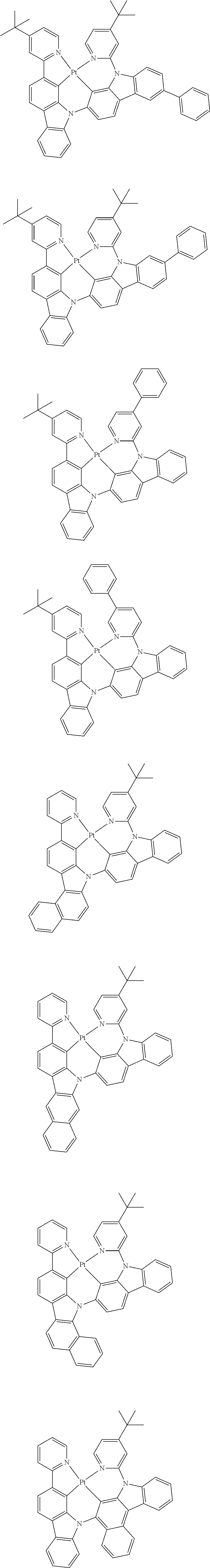

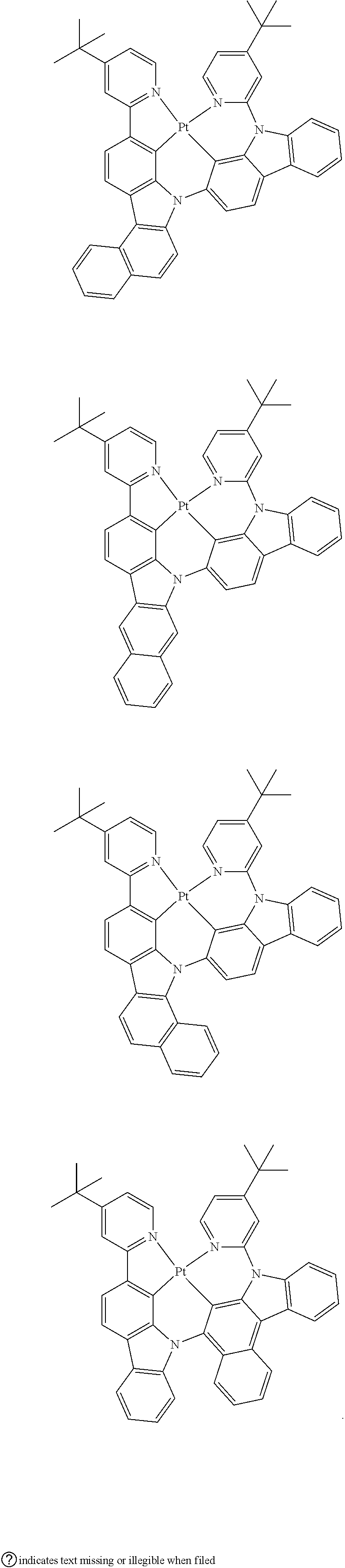

The phosphorescent material (phosphorescent compound) may be used alone or in combination of two or more phosphorescent materials.

The thickness of the emission layer is not particularly limited and may be in a range about 1 nm to about 100 nm, or for example, about 10 nm to about 30 nm.

The emission wavelength of the organic light-emitting device is not particularly limited. However, the organic light-emitting device may emit light having a peak in a wavelength region of about 360 nm or more and about 515 nm or less, about 380 nm or more and about 505 nm or less, about 400 nm or more and about 500 nm or less, about 420 nm or more and about 490 nm or less, or about 420 nm or more and about 480 nm or less.

The FWHM of an emission spectrum of the organic light-emitting device may be about 30 nm or less, about 25 nm or less, about 20 nm or less, or about 0 nm or greater.

Hereinafter, each region and each layer other than the emission layer 4 will be described in detail.

Substrate 1

The organic light-emitting device 10 may include a substrate 1. The substrate 1 may be any suitable substrate generally used in organic light-emitting devices. For example, the substrate 1 may be a glass substrate, a silicon substrate, or a transparent plastic substrate having excellent mechanical strength, thermal stability, transparency, surface smoothness, ease of handling, and water repellency, but embodiments are not limited thereto.

First Electrode 2

A first electrode 2 may be formed on the substrate 1. The first electrode 2 may be an anode and be formed of a material with a relatively high work function selected from a metal, an alloy, a conductive compound, and a combination thereof, for facilitating hole injection. The first electrode 2 may be a pixel electrode. The first electrode 2 may be a reflective electrode, a semi-transmissive electrode, or a transmissive electrode.

The materials for forming the first electrode 2 are not particularly limited and may be, for example, indium tin oxide (ITO), indium zinc oxide (IZO), tin oxide (SnO₂), zinc oxide (ZnO), indium tin zinc oxide (ITZO), or the like, having excellent transparency and conductivity, when the first electrode 2 is a transparent electrode. When the first electrode 2 is a semi-transmissive or reflective electrode, Ag, Mg, Cu, Al, Pt, Pd, Au, Ni, Nd, Ir, Cr, Li, Ca, In, LiF/Ca, LiF/Al, Mo, Ti, or a mixture thereof (e.g., a mixture of Ag and Mg or a mixture of Mg and In) may be included.

The first electrode 2 may be a single layer including a single material or a single layer including a plurality of different materials. In some embodiments, the first electrode 2 may have a multi-layer structure including a plurality of layers including various different materials.

The thickness of the first electrode 2 is not particularly limited and may be about 10 nm or greater and about 1,000 nm or lower, or about 100 nm or greater and about 300 nm or lower.

Hole Transport Region 3

A hole transport region 3 may be disposed on the first electrode 2.

The hole transport region 3 may include at least one selected from the hole injection layer 31, the hole transport layer 32, an electron blocking layer 33, and a hole buffer layer (not shown).

The hole transport region 3 may be a single layer including a single material or a single layer including a plurality of different materials. In some embodiments, the hole transport region 3 may have a multi-layer structure including a plurality of layers including various different materials.

The hole transport region 3 may include the hole injection layer 31 only or the hole transport layer 32 only. In some embodiments, the hole transport region 3 may be a single layer including a hole injection material and a hole transporting material. The hole transport region 3 may have a hole injection layer/hole transport layer structure, a hole injection layer/hole buffer layer structure, a hole injection layer/hole transport layer/hole buffer layer structure, or a hole injection layer/hole transport layer/electron blocking layer structure, wherein layers of each structure are sequentially stacked on the first electrode 2 in each stated order.

Layers forming the hole injection layer 31 and other layers included in the hole transport region 3 are not particularly limited, and a known hole injection material and/or a hole transporting material may be included.

Examples of the hole injection material may include, for example, a phthalocyanine compound such as copper phthalocyanine, N1,N1′-(biphenyl-4,4′-diyl)bis(N1-phenyl-N4,N4-di-m-tolylbenzene-1,4-diamine (DNTPD), 4,4′,4″-tris(3-methylphenylphenylamino)triphenylamine (m-MTDATA), 4,4′,4″-tris(N, N-diphenylamino))triphenylamine (TDATA), 4,4′,4″-tris{N-(2-naphthyl)-N-phenylamino}-triphenylamine (2-TNATA), poly(3,4-ethylene dioxythiophene)/poly(4-styrene sulfonate) (PEDOT/PSS), polyaniline/dodecylbenzenesulfonic acid (PANI/DBSA), polyaniline/camphor sulfonate (PANI/CSA), PANI/PSS (polyaniline)/poly(4-styrene sulfonate), N,N′-di(naphthalen-1-yl)-N,N′-diphenyl-benzidine (NPB), polyether ketone including triphenylamine (TPAPEK), 4-isopropyl-4′-methyldiphenyl iodonium tetrakis(pentafluorophenyl)borate, dipyrazino[2,3-f:2′,3′-h]quinoxaline-2,3,6,7,10,11-hexacarbonitrile (HAT-CN), and 1,3,4,5,7,8-hexafluorotetracyano-2,6-naphthoquinodimethane (F6-TCNNQ).

Examples of the hole transporting material may include a carbazole-based derivative such as N-phenylcarbazole or polyvinylcarbazole, a fluorene-based derivative, N, N′-bis(3-methylphenyl)-N, N′-diphenyl-[1),1-biphenyl]-4,4′-diamine (TPD), a triphenylamine-based derivative such as 4,4′,4″-tris(N-carbazolyl)triphenylamine (TCTA), N,N′-di(naphthalen-1-yl)-N,N′-diphenyl-benzidine (NPB), 4,4′-cyclohexylidene bis[N,N-bis(4-methylphenyl)benzene amine] (TAPC), 4,4′-bis[N, N′-(3-tril)amino]-3,3′-dimethylbiphenyl (HMTPD), 1,3-bis(N-carbazolyl)benzene (mCP), Compound H1, Compound H2, and Compound HT01:

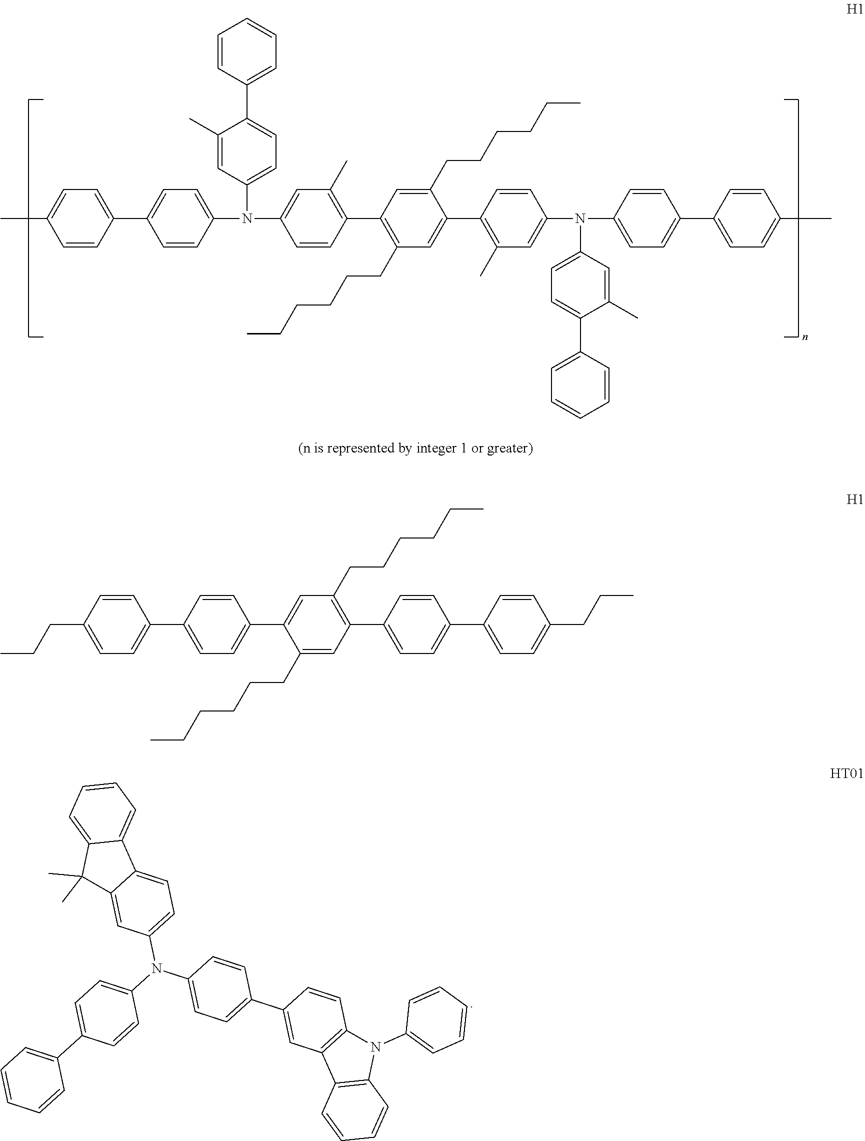

The hole transport region 3 may include a charge generating material as well as the aforementioned materials, to improve conductive properties of the hole transport region. The charge generating material may be substantially homogeneously or non-homogeneously dispersed in the hole transport region 3.

The charge generating material is not particularly limited and may be, for example, a p-dopant. Examples of the p-dopant include a quinone derivative, such as tetracyanoquinodimethane (TCNQ) or 2,3,5,6-tetrafluoro-tetracyano-1,4-benzoquinonedimethane (F4-TCNQ); a metal oxide, such as a tungsten oxide or a molybdenum oxide; and a compound containing a cyano group, but embodiments are not limited thereto:

The hole buffer layer (not shown) may increase luminescence efficiency by compensating for an optical resonance distance according to the wavelength of light emitted by the emission layer 4. Materials for forming the hole buffer layer (not shown) are not particularly limited, and a known hole buffer layer material may be used, e.g., the compounds that may be included in the hole transport region 3.

The electron blocking layer 33 may prevent electron injection from the electron transport region 5 to the hole transport region 3. Materials included in the electron blocking layer 33 are not particularly limited, and a known electron blocking layer material may be used. For example, the host materials that may be included in the emission layer and Compound H-H1 as a host material may be included.

The thickness of the hole transport region 3 is not particularly limited and may be about 1 nm or greater and about 1,000 nm or lower, or for example, about 10 nm or greater and about 500 nm or lower. In addition, the thickness of the hole injection layer 31 is not particularly limited and may be about 3 nm or greater and about 100 nm or lower. In addition, the thickness of the hole transport layer 32 is not particularly limited and may be about 3 nm or greater and about 100 nm or lower. In addition, the thickness of the electron blocking layer 33 is not particularly limited and may be about 1 nm or greater and about 100 nm or lower. In addition, the thickness of the hole buffer layer (not shown) is not particularly limited, as long as the hole buffer layer may not adversely affect on functions of an organic light-emitting device. When the thicknesses of the hole transport region 3, the hole injection layer 31, the hole transport layer 32, or the electron blocking layer 33 are within these ranges, excellent hole transport characteristics may be obtained without a substantial increase in driving voltage.

Emission Layer 4

The hole transport region 3 may be on the mission layer 4. The emission layer 4 may be understood by referring to the description of the emission layer 4 described above.

Electron Transport Region 5

The electron transport region 5 may be on the emission layer 4. The electron transport region 5 may include at least one selected from a hole blocking layer 53, the electron transport layer 52, and the electron injection layer 51.

The electron transport region 5 may be a single layer including a single material or a single layer including a plurality of different materials. In some embodiments, the electron transport region 5 may have a multi-layer structure including a plurality of layers including various different materials.

The electron transport region 5 may include the electron transport layer 52 only or the electron injection layer 51 only. In some embodiments, the electron transport region 5 may be a single layer including an electron injection material and an electron transporting material. In some embodiments, the electron transport region 5 may include an electron transport layer/electron injection layer structure or a hole blocking layer/electron transport layer/electron injection layer structure, which are sequentially stacked on the emission layer 4.

The electron injection layer 51 is not particularly limited and may include, for example, a known electron injection material. For example, the electron injection layer material may include Yb, a lithium compound, e.g., (8-hydroxyquinolinato)lithium (Liq) and lithium fluoride (LiF), sodium chloride (NaCl), cesium fluoride (CsF), rubidium fluoride (RbCl), lithium oxide (Li₂O), or barium oxide (BaO).

In some embodiments, the electron injection layer 51 may include the electron transport material and an insulating organic metal salt. The metal salt is not particularly limited and may be, for example, a material having an energy band gap or 4 eV or higher. The organic metal salt may include, for example, an acetate metal salt, a benzoate metal salt, an acetate metal salt, an acetyl acetonate metal salt, or a stearate metal salt.

The electron transport layer 52 is not particularly limited and may include, for example, a known electron transport material. Examples of the electron transport material may include an anthracene-based compound, tris(8-hydroxyquinolinolate)aluminum) (Alq3), 1,3,5-tri[(3-pyridyl)-pen-3-yl]benzene, 2,4,6-tris(3′-pyridine-3-yl)biphenyl-3-yl)-1,3,5-triazine, 2-(4-(N-phenylbenzimidazolyl-1-ylphenyl)-9,10-dinaphthylanthracene, 1,3,5-tri(1-phenyl-1H-benzo[d]imidazol-2-yl)phenyl (TPBi), 2,9-dimethyl-4,7-diphenyl-1,10-phenanthroline (BCP), 4,7-diphenyl-1,10-phenanthroline (Bphen), 3-(4-biphenyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenyl)-5-(4-tert-butylphenyl)-1,3,4-oxadiazole (tBu-PBD), bis(2-methyl-8-quinolinolate-N1,O8)-(1,1′-biphenyl-4-orato)aluminum (BAlq), berylliumbis(benzoquinoline-10-orato) (Bebq2), 9,10-di(naphthalen-2-yl)anthracene (ADN), lithum quinolate (LiQ), Compound ET1, and the like:

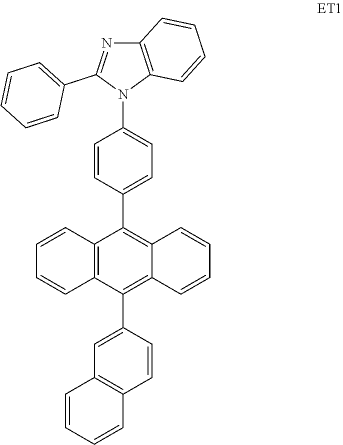 [SMILES: c1ccc(-c2nc3ccccc3n2-c2ccc(-c3c4ccccc4c(-c4ccc5ccccc5c4)c4ccccc34)cc2)cc1]

The hole blocking layer 53 may prevent hole injection from the hole transport region 3 to the electron transport region 5. Materials included in the hole blocking layer 53 is not particularly limited, and a known hole blocking material may be used. The hole blocking layer 53 may be, for example, 2,9-dimethyl-4,7-diphenyl-1,10-phenanthroline (BCP), 4,7-diphenyl-1,10-phenanthroline (Bphen), or the like. In addition, examples of the hole blocking material may include the host materials that may be included in the emission layer, and Compound H-E1 as a host material.

The thickness of the electron transport region 5 is not particularly limited and may be about 0.1 nm or greater and about 210 nm or lower, or for example, about 100 nm or greater and about 150 nm or lower. The thickness of the electron transport layer 52 is not particularly limited and may be about 10 nm or greater and about 100 nm or lower, or for example, about 15 nm or greater and about 50 nm or lower. The thickness of the hole blocking layer 53 is not particularly limited and may be about 10 nm or greater and about 100 nm or lower, or for example, about 15 nm or greater and about 50 nm or lower. The thickness of the electron injection layer 51 is not particularly limited and may be about 0.1 nm or greater and about 10 nm or lower, or for example, about 0.3 nm or greater and about 9 nm or lower. When the thickness of the electron injection layer 51 is within any of these ranges, excellent electron injection characteristics may be obtained without a substantial increase in driving voltage. When the thicknesses of the electron transport region 5, the electron injection layer 51, the electron transport layer 52, or the hole blocking layer 53 are within these ranges, excellent hole transport characteristics may be obtained without a substantial increase in driving voltage.

Second Electrode 6

A second electrode 6 may be formed on the electron injection layer 51. The second electrode 6 may be a cathode and be formed of a material with a relatively low work function selected from a metal, an alloy, or a conductive compound, for facilitating electron injection. The second electrode 6 may be a common electrode. The second electrode 6 may be a reflective electrode, a semi-transmissive electrode, or a transmissive electrode. The second electrode 6 may have a single-layered structure or a multi-layered structure including a plurality of layers. Materials for forming the second electrode 6 are not particularly limited, and for example, the second electrode 6 may include a transparent metal oxide, e.g., ITO, IZO, ZnO, or ITZO, when the second electrode 6 is a transparent electrode. When the second electrode 6 is a semi-transmissive or reflective electrode, Ag, Mg, Cu, Al, Pt, Pd, Au, Ni, Nd, Ir, Cr, Li, Ca, In, LiF/Ca, LiF/, Mo, Ti, or a mixture thereof (e.g., a mixture of Ag and Mg or a mixture of Mg and In) may be included.

The second electrode 6 may be a single layer including a single material or a single layer including a plurality of different materials. In some embodiments, the second electrode 6 may have a multi-layer structure including a plurality of layers including various different materials.

In addition, the thickness of the second electrode 6 is not particularly limited and may be about 10 nm or greater and about 1,000 nm or lower.

The second electrode 6 may be further connected to an ancillary electrode (not shown). When the second electrode 6 is connected to an ancillary electrode, a resistance of the second electrode 6 may be further reduced.

An encapsulation layer (not shown) may be further on the second electrode 6. The encapsulation layer (not shown) may be, for example, α-NPD, NPB, TPD, m-MTDATA, Alq3, CuPc, N4,N4,N4′,N4′-tetra(phenyl-4-yl)biphenyl-4,4′-diamine (TPD15), TCTA, or N, N′-bis(naphthalene-1-yl).

Furthermore, a stacking structure of the organic light-emitting device 10 according to an embodiment is not limited to the foregoing description. The organic light-emitting device 10 according to an embodiment may have a different stacking structure known in the art. For example, the organic light-emitting device 10 may not include at least one selected from the hole injection layer 31, the hole transport layer 32, the electron transport layer 52, and the electron injection layer 51 or may further include another layer. In some embodiments, each layer of the organic light-emitting device 10 may be formed as a single layer or as multiple layers.

Methods of forming each layer of the organic light-emitting device 10 according to one or more embodiments are not particularly limited. For example, vacuum-deposition, solution coating, a laser printing method, Langmuir-Blodgett (LB) method, or laser induced thermal imaging (LITI), may be used in forming each layer thereof.

The solution coating may include spin coating, casting, micro-gravure coating, gravure coating, bar coating, roll coating, wire bar coating, dip coating, spray coating, screen printing, flexographic printing, offset printing, or ink-jet printing.

The vacuum deposition may be performed at a deposition temperature in a range of about 100° C. to about 500° C., at a vacuum pressure in a range of about 10⁻⁸ torr to about 10⁻³ torr, and at a deposition rate in a range of about 0.01 nm per second (nm/sec) to about 10 nm/sec, though the conditions may vary depending on a compound that is used and a structure and thermal properties of a desired layer.

In some embodiments, the first electrode 2 may be an anode, and the second electrode 6 may be a cathode.

For example, the first electrode 2 may be an anode, the second electrode 6 may be a cathode, and an organic layer may include the emission layer 4 between the first electrode 2 and the second electrode 6 and may further include a hole transport region between the first electrode 2 and the emission layer 4 and an electron transport region between the emission layer 4 and the second electrode 6, wherein the hole transport region 3 may include at least one selected from the hole injection layer 31, the hole transport layer 32, a hole buffer layer, and an electron blocking layer, and the electron transport region 5 may include at least one selected from a hole blocking layer, the electron transport layer 52, and the electron injection layer 51.

In some embodiments, the first electrode 2 may be a cathode, and the second electrode 6 may be an anode.

In the organic light-emitting device 10 of FIGS. 1 to 3, since a voltage is applied to each of the first electrode 2 and the second electrode 6, holes provided from the first electrode 2 may move toward the emission layer 4 through the hole transport region 3, and electrons provided from the second electrode 6 may move toward the emission layer 4 through the electron transport region 5. The holes and the electrons may recombine in the emission layer 4 to produce excitons, and these excitons may transition from an excited state to a ground state to thereby generate light.

Hereinbefore, the organic light-emitting device 10 has been described with reference to FIGS. 1 to 3, but embodiments are not limited thereto.

Electronic Apparatus

The organic light-emitting device may be included in various electronic apparatuses.

The electronic apparatus may further include a thin-film transistor, in addition to the organic light-emitting device. The thin-film transistor may include a source electrode, a drain electrode, and an activation layer, wherein one of the source electrode and the drain electrode may be electrically connected to one of the first electrode and the second electrode of the organic light-emitting device.

Hereinafter, an organic light-emitting device, according to an embodiment, will be described in more detail with reference to Synthesis Examples and Examples; however, the disclosure is not limited thereto. The wording “B was used instead of A” used in describing Synthesis Examples means that an identical molar equivalent of B was used in place of A.

### EXAMPLES

### Synthesis Example 1: Synthesis of Intermediate (1)

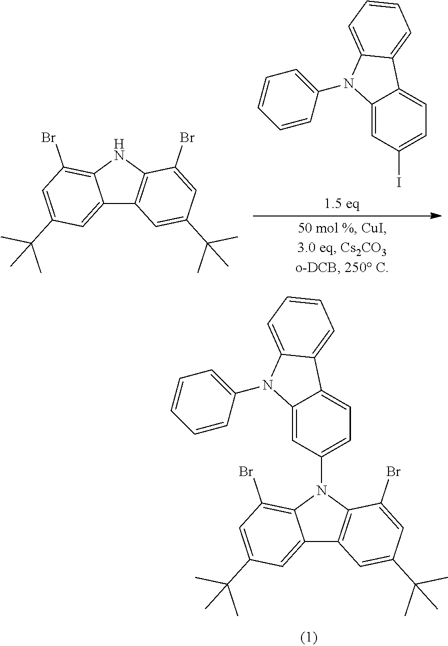 [SMILES: CC(C)(C)c1cc(Br)c2[nH]c3c(Br)cc(C(C)(C)C)cc3c2c1 ; Ic1ccc2c3ccccc3n(-c3ccccc3)c2c1 ; CC(C)(C)c1cc(Br)c2c(c1)c1cc(C(C)(C)C)cc(Br)c1n2-c1ccc2c3ccccc3n(-c3ccccc3)c2c1]

0.50 g (1.14 mmol) of 1,8-dibromo-3,6-bis(1,1-dimethylethyl)-9H-carbazole, 0.63 g (1.72 mmol) of 2-iodo-9-phenyl-9H-carbazole, 1.68 g (3.43 mmol) of cesium carbonate, and 170 mg (0.57 mmol) of copper iodide were added to a reaction tube. Then, 2.25 mL of o-dichloro benzene was added thereto, followed by heating and stirring at a temperature of 25° C. for 1 hour under microwave irradiation. After cooling to room temperature, water was added to the reaction solution. After separating the organic layer, the aqueous layer was extracted by using toluene. The organic layer was washed with saturated brine and dried using anhydrous sodium sulfate. After removing a drying agent, the organic solvent was distilled off. Intermediate (1) was obtained through purification using silica gel column chromatography (eluent: dichloromethane-hexane) (0.61 g, yield: 78%).

### Synthesis Example 2: Synthesis of Intermediate (2)

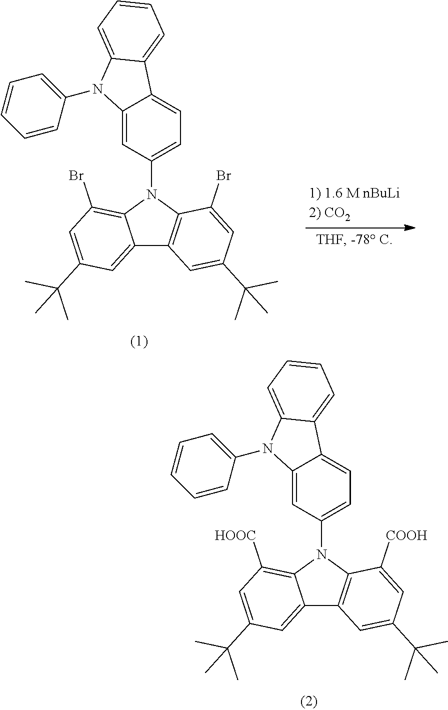 [SMILES: CC(C)(C)c1cc(Br)c2c(c1)c1cc(C(C)(C)C)cc(Br)c1n2-c1ccc2c3ccccc3n(-c3ccccc3)c2c1 ; CC(C)(C)c1cc(C(=O)O)c2c(c1)c1cc(C(C)(C)C)cc(C(=O)O)c1n2-c1ccc2c3ccccc3n(-c3ccccc3)c2c1]

1.50 g (2.46 mmol) of Intermediate (1) was added to a 100 mL 3-neck flask, followed by addition of 15 mL of THF. After cooling to a temperature of −78° C., 2.0 mL of 1.6 M n-butyllithium hexane solution (3.24 mmol, 2.2 eq.) was added dropwise thereto, followed by stirring for 1 hour. Then, dry ice was added, and the temperature was returned to room temperature, followed by stirring overnight. 20 mL of 1 M hydrochloric acid aqueous solution was added thereto. After separating the organic layer, the aqueous layer was extracted by using toluene. After washing the organic layer with water, the organic layer was dried with a drying agent (anhydrous sodium sulfate). The drying agent was separated by filtration, and the organic solvent was distilled off to obtain Intermediate (2) (0.90 g, yield: 100%).

### Synthesis Example 3: Synthesis of Compound 1-4

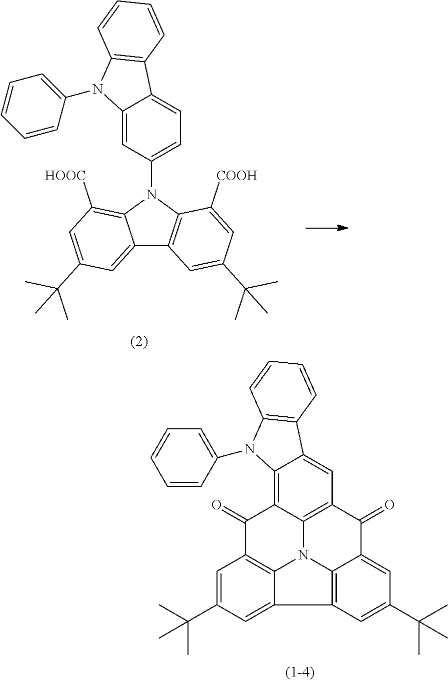 [SMILES: CC(C)(C)c1cc(C(=O)O)c2c(c1)c1cc(C(C)(C)C)cc(C(=O)O)c1n2-c1ccc2c3ccccc3n(-c3ccccc3)c2c1 ; CC(C)(C)c1cc2c(=O)c3cc4c5ccccc5n(-c5ccccc5)c4c4c(=O)c5cc(C(C)(C)C)cc6c(c1)c2n(c56)c34]

1.50 g (2.46 mmol) of Intermediate (2) was added to a 300 mL 3-neck flask, followed by adding 200 mL of dichloromethane. After adding N,N′-dimethylformamide (1 drop) and 0.95 mL (11.09 mmol) of oxalyl chloride, the mixture was heated under reflux. After 3 hours, tin (IV) tetrachloride (11.1 mL of 1 M dichloromethane solution, 11.1 mmol) was added thereto, and then the mixture was heated under reflux for additional 6 hours. After cooling to room temperature, 100 mL of 1 M aqueous hydrochloric acid solution was added. After separating an organic layer, the aqueous layer was extracted by using dichloromethane. After washing the organic layer with a saturated aqueous ammonium chloride solution, the organic layer was dried with a drying agent (anhydrous sodium sulfate). The drying agent was separated by filtration, and the organic solvent was distilled off after passing through a silica gel pad. The residue was recrystallized by using dichloromethane-methanol to obtain Compound 1-4 (1.10 g, yield: 78%).

¹H-NMR: δ 1.55 (s, 9H), 1.63 (s, 9H), 7.28-7.31 (m, 1H), 7.45-7.51 (m, 4H), 7.59-7.67 (m, 3H), 8.19 (d, 1H, J=1.2 Hz), 8.32-8.35 (m, 1H), 8.53 (d, 1H, J=1.5 Hz), 8.58 (d, 1H, J=1.2 Hz), 8.63 (d, 1H, J=1.5 Hz) 9.57 (s, 1H); LC-MS: 573 ([M+H]⁺).

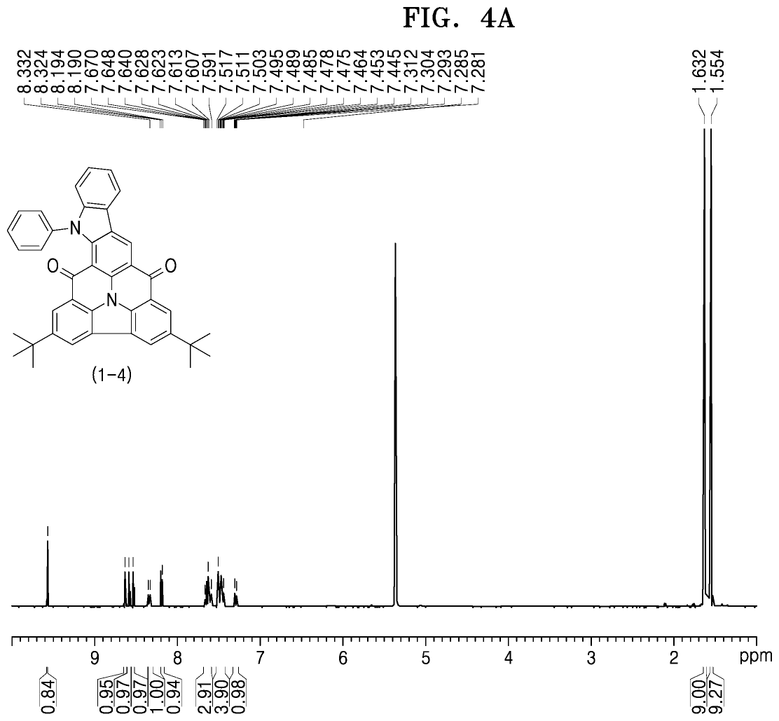

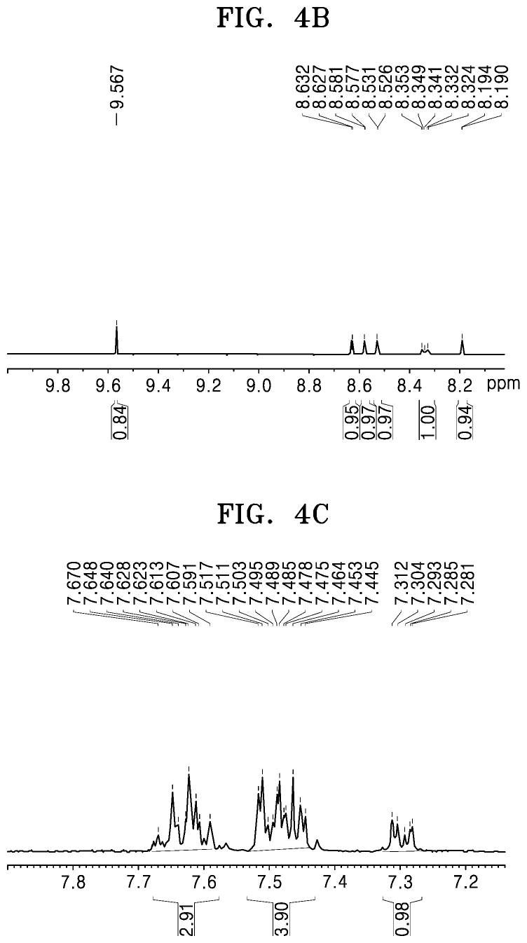

FIG. 4 shows 1H-NMR spectrum of Compound 1-3

### Synthesis Example 4: Synthesis of Intermediate (3)

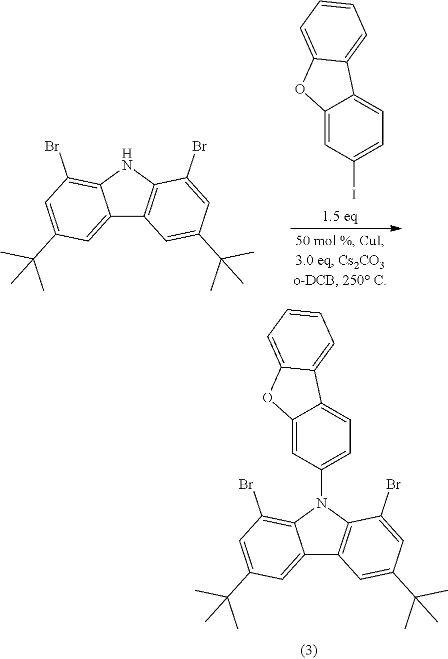 [SMILES: CC(C)(C)c1cc(Br)c2[nH]c3c(Br)cc(C(C)(C)C)cc3c2c1 ; Ic1ccc2c(c1)oc1ccccc12 ; CC(C)(C)c1cc(Br)c2c(c1)c1cc(C(C)(C)C)cc(Br)c1n2-c1ccc2c(c1)oc1ccccc12]

Intermediate (3) was obtained in the same manner as in Synthesis Example 1, except that 3.25 g (7.43 mmol) of 1,8-dibromo-3,6-bis(1,1-dimethylethyl)-9H-carbazole, 3.28 g (11.15 mmol) of 3-iodo dibenzofuran, 7.27 g (22.30 mmol) of cesium carbonate, and 710 mg (3.72 mmol) of copper iodide were added to a reaction tube, and 7.43 mL of o-dichloro benzene was added thereto (3.20 g, yield: 71%).

### Synthesis Example 5: Synthesis of Intermediate (4)

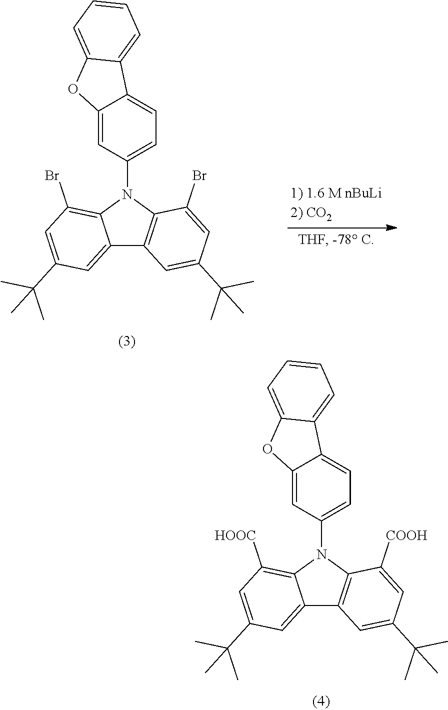 [SMILES: CC(C)(C)c1cc(Br)c2c(c1)c1cc(C(C)(C)C)cc(Br)c1n2-c1ccc2c(c1)oc1ccccc12 ; CC(C)(C)c1cc(C(=O)O)c2c(c1)c1cc(C(C)(C)C)cc(C(=O)O)c1n2-c1ccc2c(c1)oc1ccccc12]

1.00 g (1.66 mmol) of Intermediate (3) was added to a 100 mL 3-neck flask, followed by addition of 16 mL of THF. After cooling to a temperature of −78° C., 2.28 mL of 1.6 M n-butyl lithium in hexanes solution (3.65 mmol, 2.2 eq.) was added dropwise thereto, followed by stirring for 1 hour. Then, dry ice was added, and the temperature was returned to room temperature, followed by stirring overnight. After adding water, THF was concentrated, the aqueous layer was washed with hexane, and 20 mL of 1 M hydrochloric acid aqueous solution was added to the aqueous layer. After adding dichloromethane and separating an organic layer, the aqueous layer was extracted by using dichloromethane. After washing the organic layer with water, the organic layer was dried with a drying agent (anhydrous sodium sulfate). The drying agent was separated by filtration, and the organic solvent was distilled off to obtain Intermediate (4) (0.88 g, yield: 100%).

### Synthesis Example 6: Synthesis of Compound 1-5

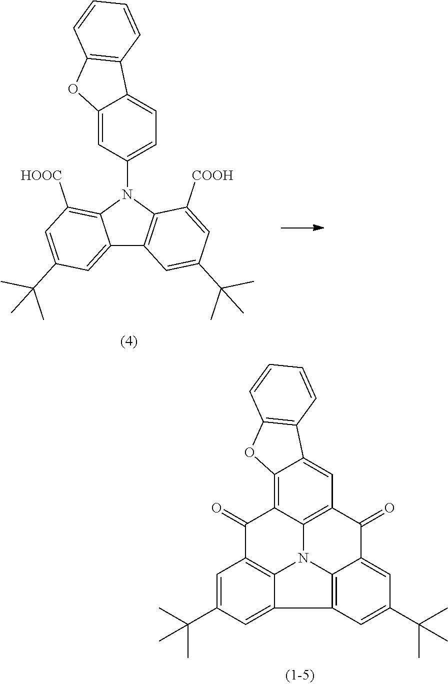 [SMILES: CC(C)(C)c1cc(C(=O)O)c2c(c1)c1cc(C(C)(C)C)cc(C(=O)O)c1n2-c1ccc2c(c1)oc1ccccc12 ; CC(C)(C)c1cc2c(=O)c3cc4c5ccccc5oc4c4c(=O)c5cc(C(C)(C)C)cc6c(c1)c2n(c56)c34]

Compound 1-5 was obtained in the same manner as in Synthesis Example 3, except that 0.70 g (1.31 mmol) of Intermediate (4) was used instead of Intermediate (2) (0.37 g, yield: 57%). LC-MS: 498 ([M+H]⁺).

### Synthesis Example 7: Synthesis of Intermediate (5)

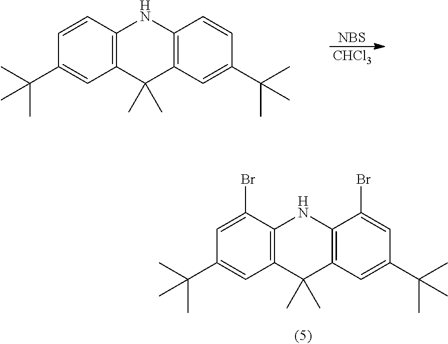

10.0 g (31.1 mmol) of 2,7-bis(1,1-dimethylethyl)-9,10-dihydro-9,9-dimethyl acridine was added to a 1 L 3-neck flask, followed by adding 500 mL of chloroform and cooling to a temperature of 0° C. 11.6 g (65.3 mmol) of NBS was added thereto, followed by stirring at a temperature of 0° C. for 30 minutes. Thereafter, the mixture was returned to room temperature and stirred for 5 hours, and 200 mL of a 20 wt % aqueous solution of sodium bisulfate was added. After separating the organic layer, the aqueous layer was extracted by using chloroform. After washing the organic layer with water, the organic layer was dried with a drying agent (anhydrous sodium sulfate). The drying agent was separated by filtration, and the organic solvent was distilled off after passing through a silica gel pad. The residue was recrystallized by using dichloromethane-methanol to obtain Intermediate (5) (10.5 g, yield: 70%).

### Synthesis Example 8: Synthesis of Intermediate (6)

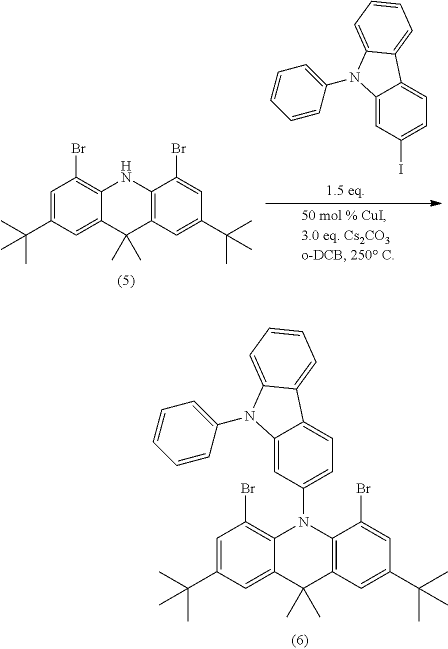 [SMILES: CC(C)(C)c1cc(Br)c2c(c1)C(C)(C)c1cc(C(C)(C)C)cc(Br)c1N2 ; Ic1ccc2c3ccccc3n(-c3ccccc3)c2c1 ; CC(C)(C)c1cc(Br)c2c(c1)C(C)(C)c1cc(C(C)(C)C)cc(Br)c1N2c1ccc2c3ccccc3n(-c3ccccc3)c2c1]

Intermediate (6) was obtained in the same manner as in Synthesis Example 1, except that 0.65 g (1.36 mmol) of Intermediate (5) was used instead of 0.50 g (1.14 mmol) of 1,8-dibromo-3,6-bis(1,1-dimethylethyl)-9H-carbazole, and 0.75 g (2.03 mmol) of 2-iodine-9-phenyl-9H-carbazole, 1.33 g (4.07 mmol) of cesium carbonate, and 130 mg (0.68 mmol) of copper iodide were added to a reaction tube, and 2.71 mL of o-dichloro benzene was added thereto (0.72 g, yield: 74%).

### Synthesis Example 9: Synthesis of Intermediate (7)

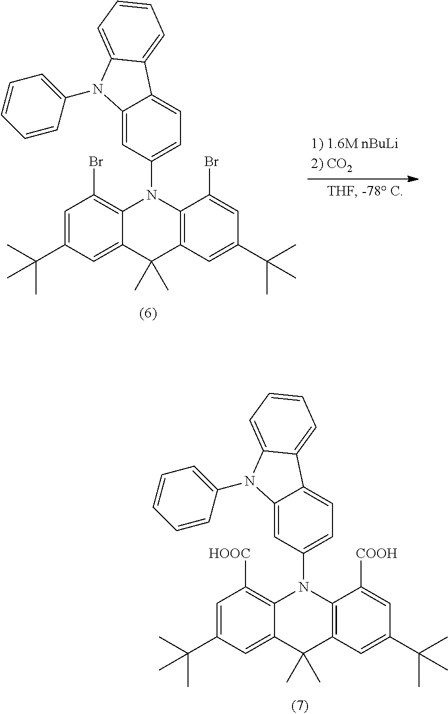 [SMILES: CC(C)(C)c1cc(Br)c2c(c1)C(C)(C)c1cc(C(C)(C)C)cc(Br)c1N2c1ccc2c3ccccc3n(-c3ccccc3)c2c1 ; CC(C)(C)c1cc(C(=O)O)c2c(c1)C(C)(C)c1cc(C(C)(C)C)cc(C(=O)O)c1N2c1ccc2c3ccccc3n(-c3ccccc3)c2c1]

1.00 g (1.39 mmol) of Intermediate (6) was added to a 100 mL 3-neck flask, followed by addition of 15 mL of THF. After cooling to a temperature of −78° C., 1.90 mL of 1.6 M n-butyl lithium in hexanes solution (3.05 mmol, 2.2 eq.) was added dropwise thereto, followed by stirring for 1 hour. Then, dry ice was added, and the temperature was returned to room temperature, followed by stirring overnight. After adding water, THF was concentrated, the aqueous layer was washed with hexane, and 20 mL of 1 M hydrochloric acid aqueous solution was added to the aqueous layer. After adding dichloromethane and separating an organic layer, the aqueous layer was extracted by using dichloromethane. After washing the organic layer with water, the organic layer was dried with a drying agent (anhydrous sodium sulfate). The drying agent was separated by filtration, and the organic solvent was distilled off to obtain Intermediate (7) (0.90 g, yield: 100%).

### Synthesis Example 10: Synthesis of Compound 1-17

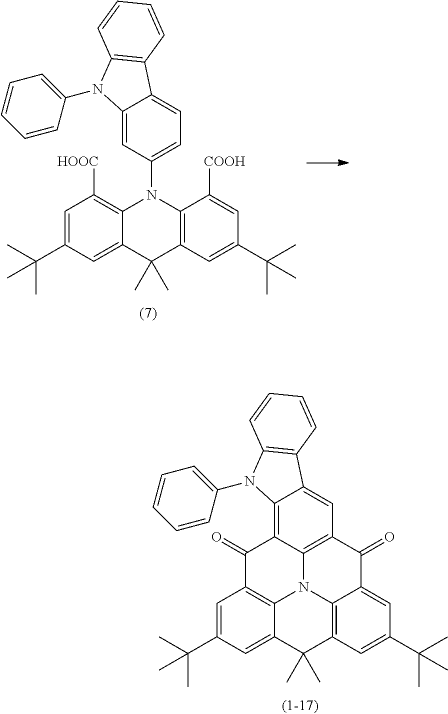 [SMILES: CC(C)(C)c1cc(C(=O)O)c2c(c1)C(C)(C)c1cc(C(C)(C)C)cc(C(=O)O)c1N2c1ccc2c3ccccc3n(-c3ccccc3)c2c1 ; CC(C)(C)c1cc2c3c(c1)c(=O)c1cc4c5ccccc5n(-c5ccccc5)c4c4c(=O)c5cc(C(C)(C)C)cc(c5n3c14)C2(C)C]

Compound 1-17 was obtained in the same manner as in Synthesis Example 3, except that 0.90 g (1.38 mmol) of Intermediate (7) was used instead of Intermediate (2) (0.44 g, yield: 52%). LC-MS: 615 ([M+H]⁺).

### Synthesis Example 11: Synthesis of Intermediate (8)

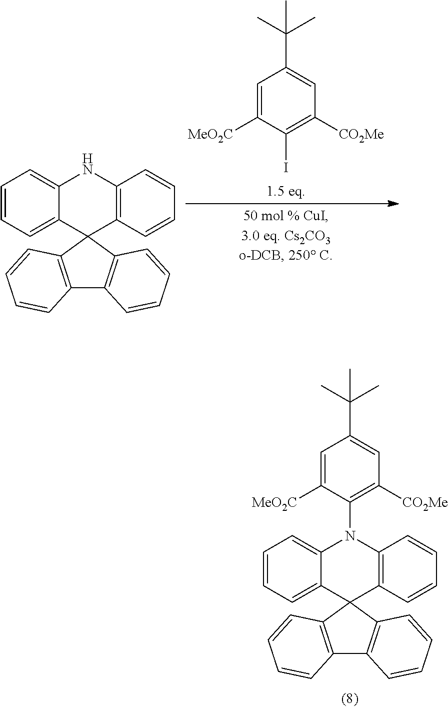 [SMILES: COC(=O)c1cc(C(C)(C)C)cc(C(=O)OC)c1I ; c1ccc2c(c1)Nc1ccccc1C21c2ccccc2-c2ccccc21 ; COC(=O)c1cc(C(C)(C)C)cc(C(=O)OC)c1N1c2ccccc2C2(c3ccccc3-c3ccccc32)c2ccccc21]

Intermediate (8) was obtained in the same manner as in Synthesis Example 1 except that 5.0 g (15 mmol) of 10H-spiro[acridine-9,9′-fluorene] was used instead of 1,8-dibromo-3,6-bis(1,1-dimethylethyl)-9H-carbazole, 8.5 g (23 mmol) of 5-tert-butyl-2-iodoisophtalate dimethyl was used instead of iodo-9-phenyl-9H-carbazole, 14.6 g (30 mmol) of cesium carbonate, and 1.4 g (7.5 mmol) of copper iodide to a reaction tube, and 25 mL of o-dichloro benzene was added thereto (0.4 g, yield: 5%).

### Synthesis Example 12: Synthesis of Intermediate (9)

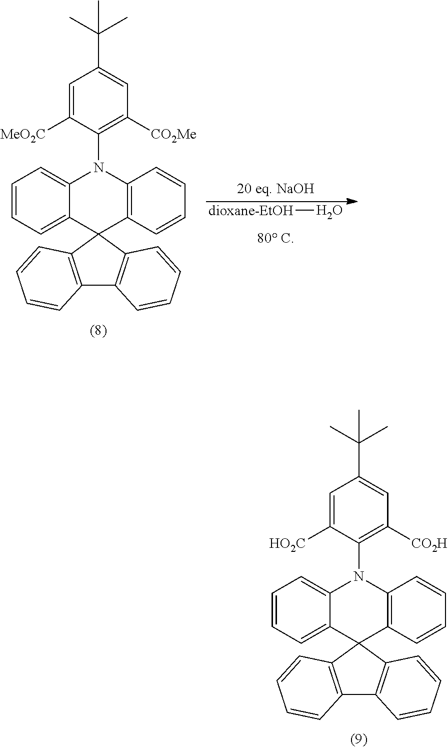

0.41 g (0.71 mmol) of Intermediate (8) and 0.56 g (14 mmol) of sodium hydroxide were added to a reaction tube, and 6 mL of dioxane, 3 mL of ethanol, and 2 mL of water were added thereto. Then, the mixture was heated up to 80° C. for 8 hours. After distilling off the solvent, 1M hydrochloric acid aqueous solution was added, and the precipitated solid was filtered to obtain Intermediate (9) (yield: 0.39 g, yield: 100%).

### Synthesis Example 13: Synthesis of Compound 2-10

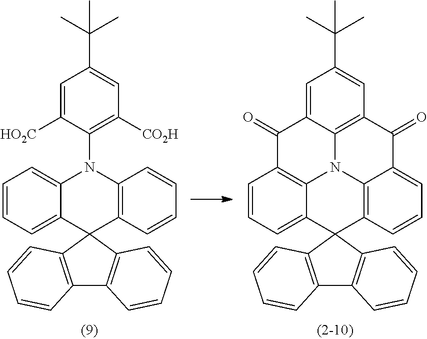 [SMILES: CC(C)(C)c1cc(C(=O)O)c(N2c3ccccc3C3(c4ccccc4-c4ccccc43)c3ccccc32)c(C(=O)O)c1 ; CC(C)(C)c1cc2c(=O)c3cccc4c3n3c5c(cccc5c(=O)c(c1)c23)C41c2ccccc2-c2ccccc21]

Compound 2-10 was obtained in the same manner as in Synthesis Example 3, except that 0.39 g (0.71 mmol) of Intermediate (9) was used instead of Intermediate (2) (0.052 g, yield: 14%). LC-MS: 516 ([M+H]⁺).

### Synthesis Example 14: Synthesis of Intermediate (10)

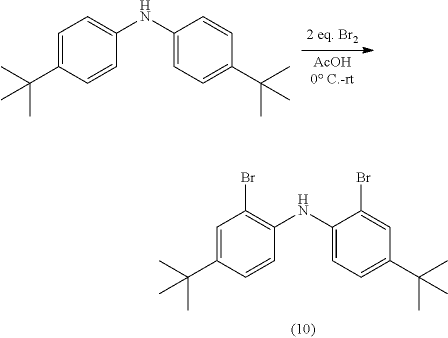

80.0 g (0.28 mol) of bis(4-tert-butylphenyl)amine was added to a 3 L 3-neck flask, followed by adding 1 L of acetic acid and cooling to a temperature of 0° C. A solution in which 91 g (0.57 mol) of bromine was diluted in 200 mL of acetic acid was added dropwise thereto at a temperature of 0° C. for an hour and a half. Then, the temperature was returned to room temperature, followed by stirring overnight. After adding 1 L of 1 wt % aqueous solution of sodium thiosulfate, the precipitated solid was filtered out. The residue was recrystallized by using dichloromethane-methanol to obtain Intermediate (10) (82 g, yield: 66%).

### Synthesis Example 15: Synthesis of Intermediate (11)

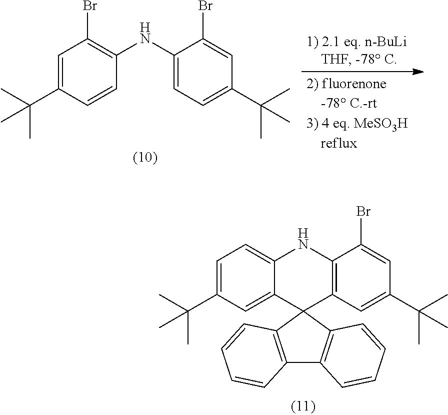

20.0 g (0.046 mol) of Intermediate (10)(bis(2-bromo-4-tert-butylphenyl)amine) was added to a 1 L 3-neck flask, followed by adding 0.5 L of THF. Then, the temperature was cooled to −78° C. n-butyllithium (1.6 M in hexanes solution, 0.57 mol, 60 mL) was added dropwise to, followed by stirring at a temperature of −78° C. for an 1 hour and a half. After adding 8.3 g (0.046 mmol) of fluorenone, the temperature was gradually raised to room temperature, followed by stirring at room temperature for 16 hours. A solution diluted in 200 mL of acetic acid was added dropwise thereto at a temperature of 0° C. for an hour and a half. Then, the temperature was returned to room temperature, followed by stirring overnight. After adding 1 L of 1 wt % aqueous solution of sodium thiosulfate, the precipitated solid was filtered. The residue was recrystallized by using dichloromethane-methanol to obtain Intermediate (11) (82 g, yield: 66%).

### Synthesis Example 16: Synthesis of Intermediate (12)

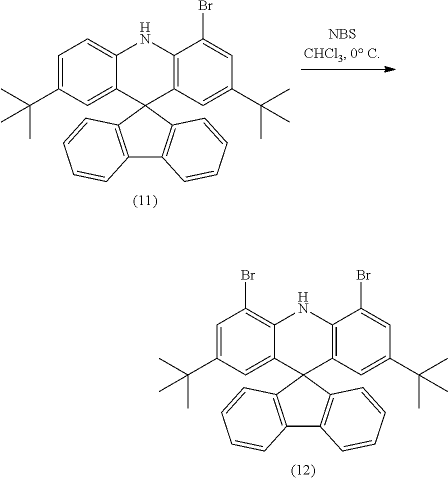

9.4 g (18 mmol) of Intermediate (11) was added to a 500 mL 3-neck flask, followed by adding 200 mL of chloroform and cooling to a temperature of 0° C. 3.2 g (18 mmol) of NBS was added thereto, followed by stirring at a temperature of 0° C. for 30 minutes. Thereafter, the mixture was returned to room temperature and stirred for 5 hours, and 200 mL of a 20 wt % aqueous solution of sodium hydrogen sulphate was added. After separating the organic layer, the aqueous layer was extracted by using chloroform. After washing the organic layer with water, the organic layer was dried with a drying agent (anhydrous sodium sulfate). The drying agent was separated by filtration, and the organic solvent was distilled off after passing through a silica gel pad. The residue was recrystallized by using chloroform-methanol to obtain Intermediate (12) (10.2 g, yield: 94%).

### Synthesis Example 17: Synthesis of Intermediate (13)

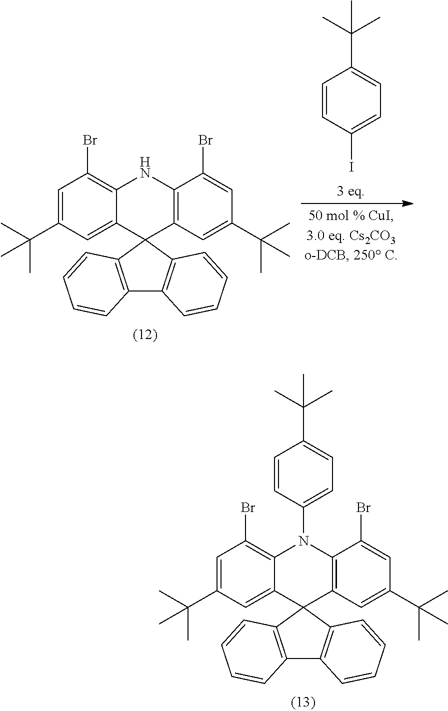

Intermediate (13) was obtained in the same manner as in Synthesis Example 1, except that 2.0 g (3.3 mmol) of Intermediate (12), 2.6 g (10 mmol) of 4-tert-butyliodine benzene, 3.3 g (10 mmol) of cesium carbonate, and 320 mg (1.6 mmol) of copper iodide were added to a reaction tube, and 5.4 mL of o-dichloro benzene was added thereto (2.0 g, yield: 82%).

### Synthesis Example 18: Synthesis of Intermediate (14)

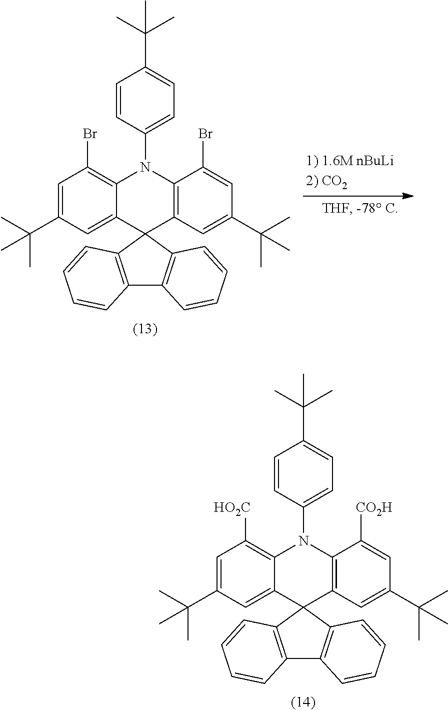

2.0 g (2.7 mmol) of Intermediate (13) was added to a 100 mL 3-neck flask, followed by addition of 50 mL of THF. After cooling to a temperature of −78° C., 3.8 mL of 1.6 M n-butyl lithium in hexanes solution (6.0 mmol, 2.2 eq.) was added dropwise thereto, followed by stirring for 1 hour. Then, dry ice was added, and the temperature was returned to room temperature, followed by stirring overnight. After adding water, THF was concentrated, the aqueous layer was washed with hexane, and 20 mL of 1 M hydrochloric acid aqueous solution was added to the aqueous layer. After adding dichloromethane and separating an organic layer, the aqueous layer was extracted by using dichloromethane.

After washing the organic layer with water, the organic layer was dried with a drying agent (anhydrous sodium sulfate). The drying agent was separated by filtration, and the organic solvent was distilled off to obtain Intermediate (14) (1.1 g, yield: 57%).

### Synthesis Example 19: Synthesis of Compound 2-11

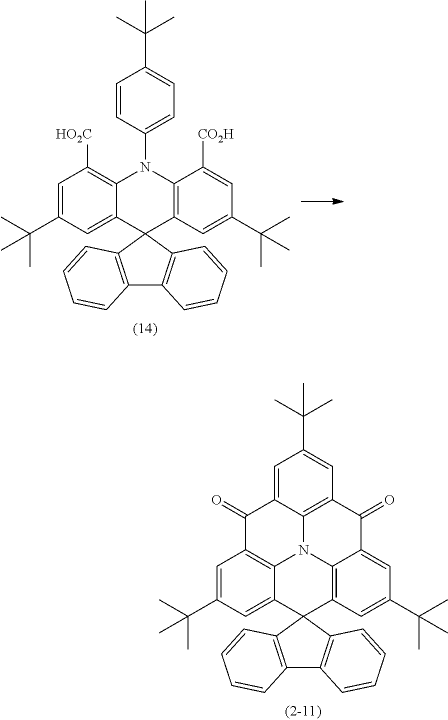

Compound 2-11 was obtained in the same manner as in Synthesis Example 3, except that 1.0 g (1.5 mmol) of Intermediate (14) was used (0.52 g, yield: 54%). LC-MS: 628 ([M+H]⁺).

### Synthesis Example 20: Synthesis of Intermediate (15)

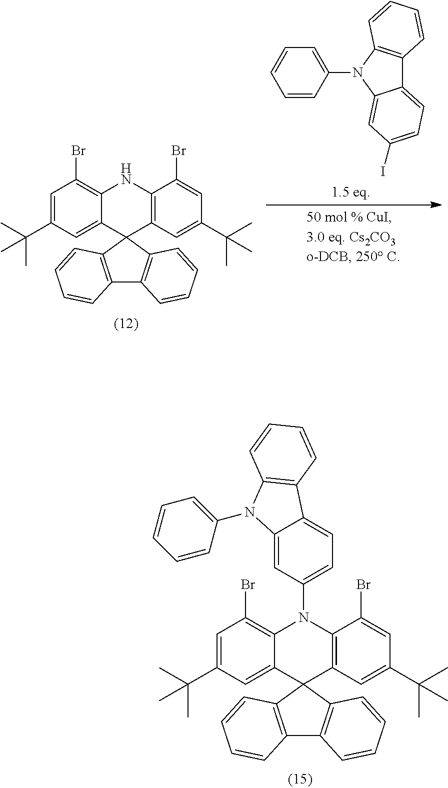 [SMILES: CC(C)(C)c1cc(Br)c2c(c1)C1(c3ccccc3-c3ccccc31)c1cc(C(C)(C)C)cc(Br)c1N2 ; Ic1ccc2c3ccccc3n(-c3ccccc3)c2c1 ; CC(C)(C)c1cc(Br)c2c(c1)C1(c3ccccc3-c3ccccc31)c1cc(C(C)(C)C)cc(Br)c1N2c1ccc2c3ccccc3n(-c3ccccc3)c2c1]

Intermediate (15) was obtained in the same manner as in Synthesis Example 1, except that 2.00 g (3.33 mmol) of Intermediate (12), 1.84 g (4.99 mmol) of 2-iodine-9-phenyl-9H-carbazole, 3.25 g (9.98 mmol) of cesium carbonate, and 320 mg (1.66 mmol) of copper iodide were added to a reaction tube, and 4.75 mL of o-dichloro benzene was added thereto (2.30 g, yield: 82%).

### Synthesis Example 21: Synthesis of Intermediate (16)

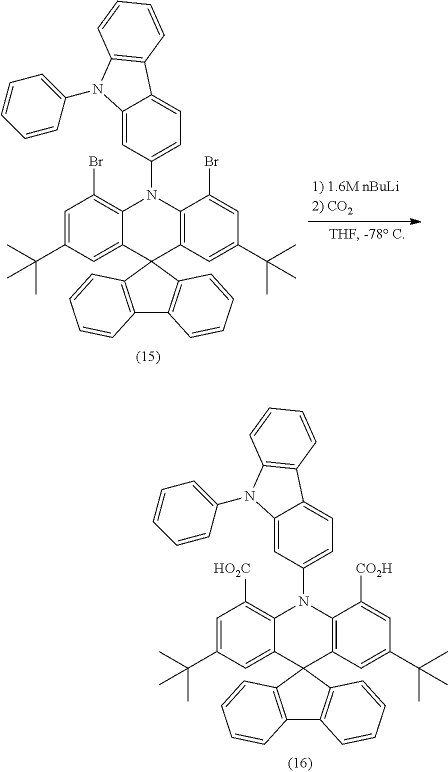 [SMILES: CC(C)(C)c1cc(Br)c2c(c1)C1(c3ccccc3-c3ccccc31)c1cc(C(C)(C)C)cc(Br)c1N2c1ccc2c3ccccc3n(-c3ccccc3)c2c1 ; CC(C)(C)c1cc(C(=O)O)c2c(c1)C1(c3ccccc3-c3ccccc31)c1cc(C(C)(C)C)cc(C(=O)O)c1N2c1ccc2c3ccccc3n(-c3ccccc3)c2c1]

2.30 g (2.73 mmol) of Intermediate (15) was added to a 100 mL 3-neck flask, followed by addition of 27 mL of THF. After cooling to a temperature of −78° C., 3.75 mL of 1.6 M n-butyl lithium in hexane solution (6.0 mmol, 2.2 eq.) was added dropwise thereto, followed by stirring for 1 hour. Then, dry ice was added, and the temperature was returned to room temperature, followed by stirring overnight. After adding water, THF was concentrated, the aqueous layer was washed with hexane, and 20 mL of 1 M hydrochloric acid aqueous solution was added to the aqueous layer. After adding dichloromethane and separating an organic layer, the aqueous layer was extracted by using dichloromethane. After washing the organic layer with water, the organic layer was dried with a drying agent (anhydrous sodium sulfate). The drying agent was separated by filtration, and the organic solvent was distilled off to obtain Intermediate (16) (2.11 g, yield: 100%).

### Synthesis Example 22: Synthesis of Compound 2-24

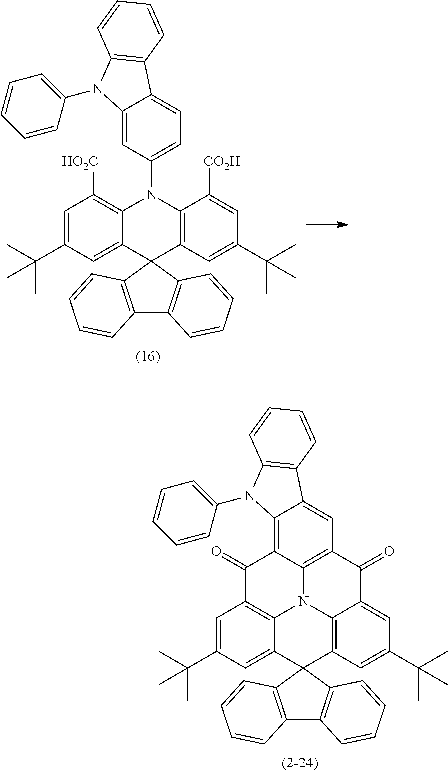 [SMILES: CC(C)(C)c1cc(C(=O)O)c2c(c1)C1(c3ccccc3-c3ccccc31)c1cc(C(C)(C)C)cc(C(=O)O)c1N2c1ccc2c3ccccc3n(-c3ccccc3)c2c1 ; CC(C)(C)c1cc2c3c(c1)c(=O)c1cc4c5ccccc5n(-c5ccccc5)c4c4c(=O)c5cc(C(C)(C)C)cc(c5n3c14)C21c2ccccc2-c2ccccc21]

Compound 2-24 was obtained in the same manner as in Synthesis Example 3, except that 2.11 g (2.73 mmol) of Intermediate (16) was used (1.40 g, yield: 70%). LC-MS: 737 ([M+H]⁺).

[Other Condensed Cyclic Compounds According to Embodiments]

Condensed cyclic compounds according to embodiments other than the above compounds also have a skeletal structure common to each compound shown above or a similar skeletal structure, so in each synthesis method, other condensed cyclic compounds according to embodiments may be synthesized by appropriately changing raw materials, reaction conditions, and the like to be used, or by appropriately combining the above synthetic methods and known synthetic methods.

[Simulation Evaluation of Condensed Cyclic Compounds]

According to “High-Performance Dibenzoheteraborin-Based Thermally Activated Delayed Fluorescence Emitters: Molecular Architectonics for Concurrently Achieving Narrowband Emission and Efficient Triplet-Singlet Spin Conversion” In Seob Park, Kyohei Matsuo, Naoya Aizawa, and Takuma Yasuda, Advanced Functional Materials 2018, 28, 1802 031, the fluorescence spectral width (full width at half maximum, FWHM) was found to be closely related with the rearrangement energy [E(S₀@S₁)−E(S₀@ S₀)] expressed as the difference from the energy [E(S₀@S₁)] of the ground state (S₀) in a stable structure of a first excited singlet state (S₁) and the energy [E(S₀@S₀)] of the ground state (S₀) in a stable structure of the ground state (S₀).

Confirmation of Relationship Between Rearrangement Energy and Fluorescence Spectrum Width

First, as shown in the following, the relationship between the rearrangement energy [E(S₀@S₁)−E(S₀@S₀)] and the fluorescence spectral width (FWHM) was identified.

Calculation by Density Functional Theory (DFT)

The following calculation was performed for the known condensed cyclic compounds R¹ to R³ described below by DFT.

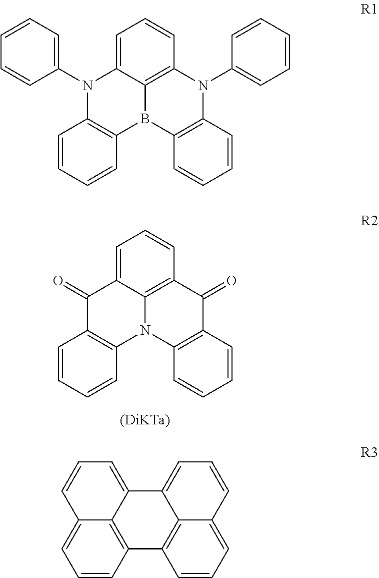

The rearrangement energy [E(S₀@S₁)]−[E(S₀@S₀)](eV) was calculated from the difference of the energy [E(S₀@S₁)] of the ground state (S₀) in a stable structure of a first excited singlet state (S₁) and the energy [E(S₀@S₀)] of the ground state (S₀) in a stable structure of the ground state (S₀).

The adiabatic first excited singlet state (S₁) energy [E(S₁@S₁)]−[E(S₀@S₀)] (eV) was calculated from the difference between the calculated energy [E(S₁@S₁)] of the first excited singlet state (S₁) in a stable structure of the first excited singlet state (S₁) and the energy [E(S₀@S₀)] of the ground state (S₀) in a stable structure of the ground state (S₀).

Then, the fluorescence wavelength (nm) was calculated by converting the adiabatic first excited singlet state (S₁) energy (eV) into the optical wavelength (nm).

In addition, the oscillator intensity f in the stable structure of the first excited singlet state (S₁) was calculated.

In addition, the highest occupied molecular orbital (HOMO) energy and the lowest unoccupied molecular orbital (LUMO) energy were calculated.

Here, the calculation by DFT was performed using Gaussian 16 (Gaussian Inc.) as the calculation software, and the following (I), (II) and (III) calculation methods were used:
(I) S₀ calculation method: structural optimization calculation by DFT including functional B3LYP, basis function 6-31G(d, p), and toluene solvent effect (PCM);
(II) S₁ calculation method: structural optimization calculation by DFT including functional B3LYP, basis function 6-31G(d, p), and time-dependent DFT (TDDFT);
(III) S₀ calculation method: structural optimization calculation by DFT including functional B3LYP, basis function 6-31G(d, p), and the input structure by DFT including toluene solvent effect (PCM);
In detail, the calculation of each item was performed using the following calculation methods.
the energy [E(S₀@S₀)] of the ground state (S₀) in a stable structure of the ground state (S₀): the (I) calculation method;
the energy [E(S₁@S₁)] of the first excited singlet state (S₁) in a stable structure of the excited singlet state (S₁): the (II) calculation method;
the energy [E(S₀@S₁)] of the ground state (S₀) in a stable structure of the first excited singlet state (S₁): the (II) and (III) calculation methods;
the rearrangement energy [E(S₀@S₁)]−[E(S₀@S₀)]: the (I), (II), and (III) calculation methods;
the adiabatic first excited singlet state energy [E(S₁@S₁)]−[E(S₀@S₀)]: the (I) and (II) calculation methods;
the fluorescence wavelength (nm): the (I) and (II) calculation methods;
the oscillator intensity fin a stable structure of the first excited singlet state (S₁): the (II) calculation method; and
HOMO and LUMO: the (I) calculation method.

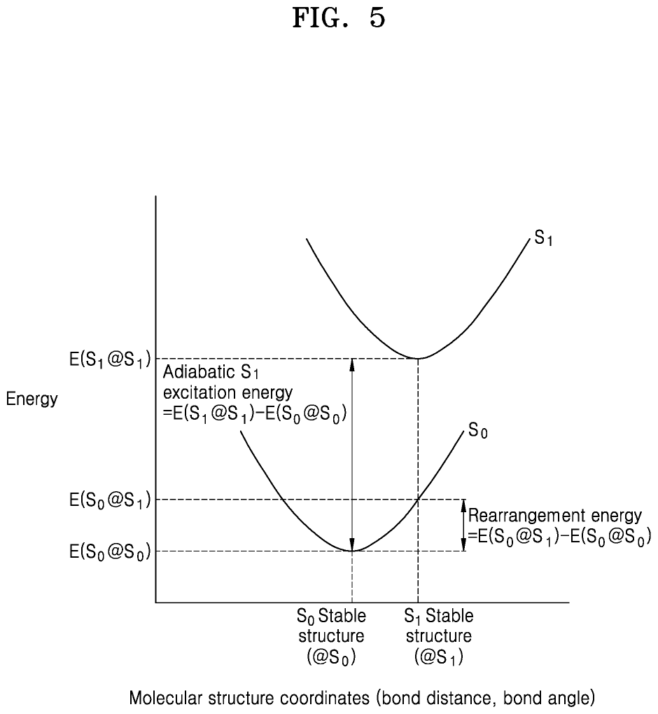

FIG. 5 is a view qualitatively illustrating an energy relationship.

The results of evaluation are shown in Table 1.

### Measurement of Fluorescence Spectral Width (FWHM)

With respect to each 1×10⁻⁵ M (=mol/dm³, mol/L) toluene solution of condensed cyclic compounds R1 to R3, measurement was performed at room temperature with an excitation wavelength of 320 nm by spectrofluorescence photometer F7000 manufactured by Hitachi High-Tech Science Co., Ltd., thereby evaluating the fluorescence emission peak wavelength (nm) and fluorescence spectral width (FWHM) in photoluminescence (PL).

The results of evaluation are shown in Table 1.

TABLE 1

| | Calculation by density functional method | | | | | | Peak Wavelength | |
|---|---|---|---|---|---|---|---|---|
| | | | Adiabatic excited | | Rearrangement | Fluorescence | PL Peak | |
| | HOMO | LUMO | singlet state (S₁) | Oscillator | Energy | Wavelength | Wavelength | PL EWHM |
| | (eV) | (ev) | energy (eV) | Intensity | (eV) | (nm) | (nm) | (nm) |
| R1 | −4.88 | −1.23 | 2.99 | 0.214 | 0.109 | 415 | 453 | 22 |
| R2 | −5.94 | −2.36 | 2.96 | 0.161 | 0.132 | 419 | 451 | 26 |
| R3 | −5.02 | −1.96 | 2.62 | 0.491 | 0.164 | 474 | 445 | 42 |

From the results of Table 1, it was confirmed that the color of the fluorescence wavelength (nm) calculated by the DFT and the measured peak wavelength show values close to some extent. Accordingly, it was confirmed that the color estimated by the calculation by the DFT and the actual color were the same color.

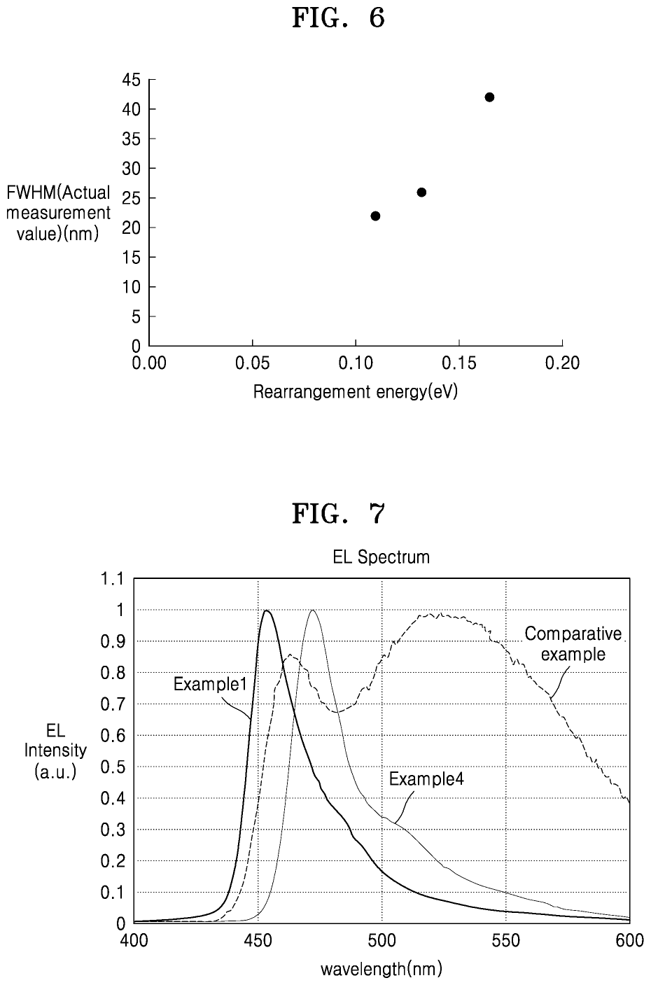

Here, a graph of the rearrangement energy (eV) calculated by the fluorescence FWHM-DFT in the measured PL of the condensed cyclic compounds R1 to R3 is shown in FIG. 6. As shown in FIG. 6, the rearrangement energy (eV) calculated by the DFT and the fluorescence FWHM are related, and the smaller the rearrangement energy (eV), the smaller the fluorescence FWHM, that is, it was confirmed that the fluorescence spectral width was narrowed.

### Calculation of Rearrangement Energy and Fluorescence Wavelength of Compounds According to Embodiments and Comparative Compound R2

For Compounds 1-4, 1-5, and 1-17 and Comparative Compound R2 according to embodiments, HOMO (eV), rearrangement energy (eV), fluorescence wavelength (nm) and FWHM were calculated in the same manner as in the method described in [Confirmation of relationship between rearrangement energy and fluorescence spectral width]. The results thereof are shown in Table 2.

For Compounds 2-24, 2-11, 2-10, and 2-19 and Comparative Compound R2 according to embodiments, HOMO (eV), rearrangement energy (eV), fluorescence wavelength (nm) and FWHM were calculated in the same manner as in the method described in [Confirmation of relationship between rearrangement energy and fluorescence spectral width]. The results thereof are shown in Table 3.

TABLE 2

| | | | | Fluorescence | |
|---|---|---|---|---|---|
| | | HOMO | Rearrangement | Wavelength | |
| Compound | Structure | (eV) | Energy (eV) | (nm) | FWHM |
| 1-4 | 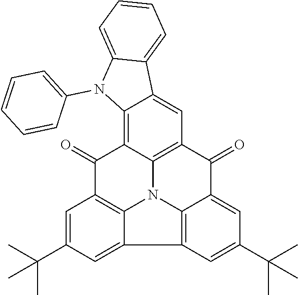 [SMILES: CC(C)(C)c1cc2c(=O)c3cc4c5ccccc5n(-c5ccccc5)c4c4c(=O)c5cc(C(C)(C)C)cc6c(c1)c2n(c56)c34] | −5.50 | 0.085 | 429 | 22 |
| 1-5 | 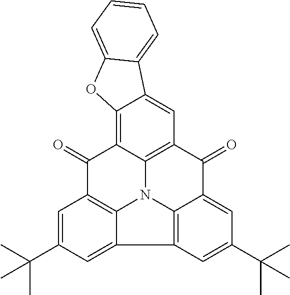 [SMILES: CC(C)(C)c1cc2c(=O)c3cc4c5ccccc5oc4c4c(=O)c5cc(C(C)(C)C)cc6c(c1)c2n(c56)c34] | −5.68 | 0.070 | 428 | 19 |
| 1-17 | 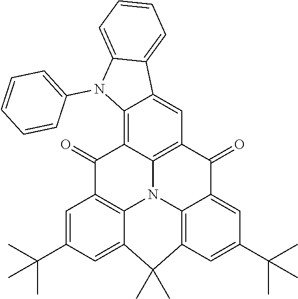 [SMILES: CC(C)(C)c1cc2c3c(c1)c(=O)c1cc4c5ccccc5n(-c5ccccc5)c4c4c(=O)c5cc(C(C)(C)C)cc(c5n3c14)C2(C)C] | −5.36 | 0.076 | 451 | 25 |
| R2 (DiKTa) | 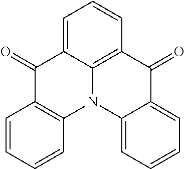 [SMILES: O=c1c2ccccc2n2c3ccccc3c(=O)c3cccc1c32] | −5.94 | 0.132 | 438 | 44 |

TABLE 3

| | | | | Fluorescence | |
|---|---|---|---|---|---|
| | | HOMO | Rearrangement | Wavelength | |
| Compound | Structure | (ev) | Energy (eV) | (nm) | FWHM |
| 2-24 | 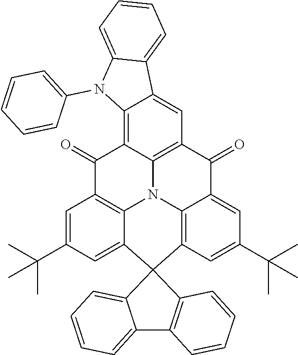 [SMILES: CC(C)(C)c1cc2c3c(c1)c(=O)c1cc4c5ccccc5n(-c5ccccc5)c4c4c(=O)c5cc(C(C)(C)C)cc(c5n3c14)C21c2ccccc2-c2ccccc21] | −5.37 | 0.075 | 450 | 26 |
| 2-11 | 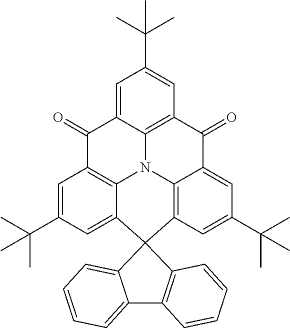 [SMILES: CC(C)(C)c1cc2c3c(c1)c(=O)c1cc(C(C)(C)C)cc4c(=O)c5cc(C(C)(C)C)cc(c5n3c14)C21c2ccccc2-c2ccccc21] | −5.56 | 0.098 | 449 | 23 |
| 2-10 | 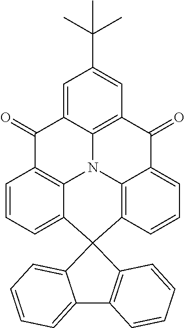 [SMILES: CC(C)(C)c1cc2c(=O)c3cccc4c3n3c5c(cccc5c(=O)c(c1)c23)C41c2ccccc2-c2ccccc21] | −5.63 | 0.076 | 455 | 30 |
| 2-19 | 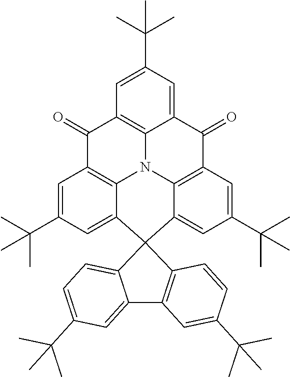 [SMILES: CC(C)(C)c1ccc2c(c1)-c1cc(C(C)(C)C)ccc1C21c2cc(C(C)(C)C)cc3c(=O)c4cc(C(C)(C)C)cc5c(=O)c6cc(C(C)(C)C)cc1c6n(c23)c45] | −5.50 | 0.104 | 452 | 23 |
| R2 (DiKTa) | 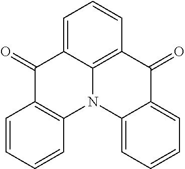 [SMILES: O=c1c2ccccc2n2c3ccccc3c(=O)c3cccc1c32] | −5.94 | 0.132 | 438 | 44 |

As shown in Tables 2 and 3, the compounds according to embodiments each had a rearrangement energy of 0.110 eV or less, which is smaller than that of Comparative Compound R2. Therefore, referring to FIG. 6, it is estimated that the FWHM of each of the compounds according to embodiments is smaller than that of Comparative Compound R2, which is known in the art. Therefore, the compounds according to embodiments are considered to exhibit significantly higher color purity than Comparative Compound R2.

The compounds according to embodiments each had a fluorescence wavelength of 425 nm or more and 480 nm or less, and it was confirmed that the compounds each have an appropriate blue fluorescence wavelength.

From the above results, it can be seen that the compounds according to embodiments each had a small rearrangement energy. Therefore, it was confirmed that the compound of the compounds according to embodiments are expected as a blue light-emitting material capable of realizing a narrow emission spectrum and high color purity.

Evaluation of Condensed Cyclic Compound: Measurement of Fluorescence Spectral Width (FWHM)

With respect to each 1×10⁻⁷ M (=mol/dm³, mol/L) toluene solution of Compound 1-4 and 1-5, measurement was performed at room temperature with an excitation wavelength of 320 nm by spectrofluorescence photometer F7000 manufactured by Hitachi High-Tech Science Co., Ltd., thereby evaluating the fluorescence emission peak wavelength (nm) and fluorescence spectral width (FWHM) in photoluminescence (PL). The results thereof are shown in Table 4. In addition, Table 4 also shows the measurement results of the condensed cyclic compound R² measured in the same manner as above.

With respect to each 2×10⁻⁷ M (=mol/dm³, mol/L) toluene solution of Compound 2-10, 2-11, and 2-24, measurement was performed at room temperature with an excitation wavelength of 320 nm by spectrofluorescence photometer F7000 manufactured by Hitachi High-Tech Science Co., Ltd., thereby evaluating the fluorescence emission peak wavelength (nm) and fluorescence spectral width (FWHM) in photoluminescence (PL). The results thereof are shown in Table 5. In addition, Table 5 also shows the measurement results of the condensed cyclic compound R2 measured in the same manner as above.

In addition, in this evaluation, the peak wavelength of fluorescence emission may be, but is not particularly limited to, within the blue emission region, and specifically may be about 440 nm or more and about 470 nm or less.

In this evaluation, it is preferable that the spectral width FWHM of fluorescence emission is smaller, and the small spectral width FWHM may be judged as high color purity.

TABLE 4

| | PL peak | PL | |
|---|---|---|---|
| | wavelength | FWHM | |
| Compound | (nm) | (nm) | |
| Compound 1-4 | 443 | 14 | the present invention |
| Compound 1-5 | 442 | 15 | the present invention |
| Comparative | 449 | 26 | known condensed |
| compound R2 | | | cyclic compound |

TABLE 5

| | PL peak | PL | |
|---|---|---|---|
| | wavelength | FWHM | |
| Compound | (nm) | (nm) | |
| Compound 2-10 | 459 | 22 | the present invention |
| Compound 2-11 | 463 | 19 | the present invention |
| Compound 2-24 | 457 | 18 | the present invention |
| Comparative | 449 | 26 | known condensed |
| compound R2 | | | cyclic compound |

### Example 1: Manufacture of Organic Light-Emitting Device

An ITO glass substrate was cut to a size of 50 millimeters (mm)×50 mm×0.5 mm. Then the glass substrate was sequentially sonicated in acetone, isopropyl alcohol, and pure water for about 15 minutes in each solvent, and cleaned by exposure to ultraviolet rays (UV) with ozone for 30 minutes. A next layer was deposited on the ITO electrode (anode) on the glass substrate with a vacuum deposition apparatus.

First, HAT-CN (manufactured by e-Ray) was deposited on the ITO electrode to form a hole injection layer having a film thickness of 10 nm. Subsequently, Compound HT01 was deposited on the hole injection layer to form a hole transport layer having a thickness of 140 nm. Subsequently, Compound H-H1 was deposited on the hole transport layer to form an electron blocking layer having a thickness of 5 nm to thereby form a hole transport region.

Compound H-H1 as a host material (HT-Host Compound) having hole transportability, Compound H-E1 as a host material (ET-Host Compound) having electron transportability, and Compound 1-4 obtained as above were co-deposited on the hole transport region to form an emission layer having a film thickness of 40 nm.

Here, the mass ratio of Compound H-H1 to Compound H-E1 in the emission layer was as shown in Table 6.

Compound H-E1 was vacuum-deposited on the emission layer to form a hole blocking layer having a film thickness of 5 nm. Subsequently, TRE314 (as an electron transport material manufactured by Toray Co., Ltd.) and LiQ were co-deposited at a mass ratio of 5:5 (parts by weight) on the hole blocking layer to form an electron transport layer having a film thickness of 30 nm. Subsequently, LiQ was deposited on the electron transport layer to form an electron injection layer having a film thickness of 1 nm to thereby form an electron transport region.

Al (cathode) was deposited on the electron injection layer to a film thickness of 100 nm, thereby manufacturing an organic light-emitting device.

The manufacture was performed by using a glass sealant tube containing a drying agent and an ultraviolet curable resin (product name WB90U manufactured by MORESCO) in a glove box under nitrogen atmosphere at a moisture concentration of 1 part per million (ppm) or less and at an oxygen concentration of 1 ppm or less. The organic light-emitting device was sealed to thereby complete the manufacture of organic light-emitting device.

The structures of Compounds HT01, H-H1 and H-E1 are as follows:

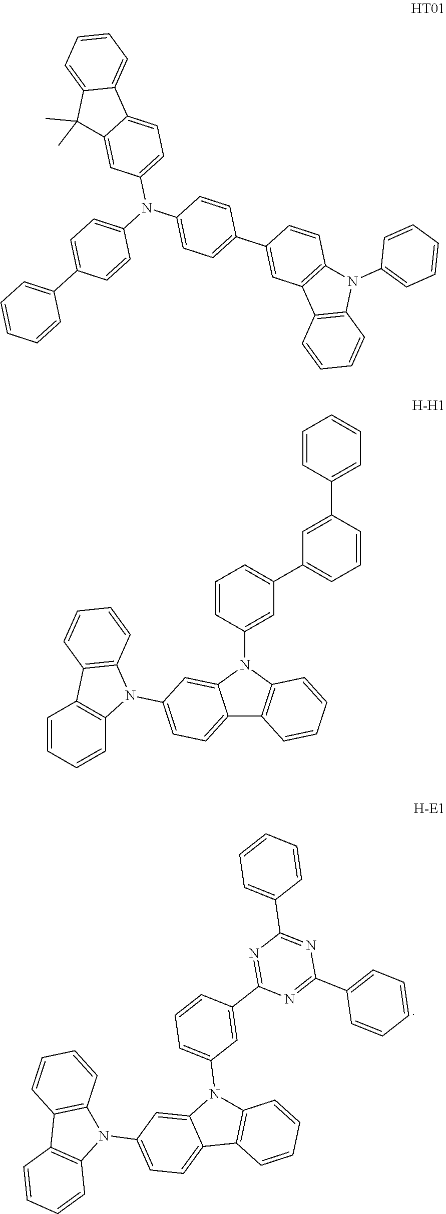 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2cccc(-c3cccc(-n4c5ccccc5c5ccc(-n6c7ccccc7c7ccccc76)cc54)c3)c2)cc1 ; c1ccc(-c2nc(-c3ccccc3)nc(-c3cccc(-n4c5ccccc5c5ccc(-n6c7ccccc7c7ccccc76)cc54)c3)n2)cc1]

The CIEx, CIEy, emission peak wavelength, and full width FWHM were evaluated according to the following methods.

The organic light-emitting device was allowed to emit light by continuously changing the voltage applied to the organic light-emitting device using a DC constant voltage power supply (2400 source meter from KEITHLEY), and the luminance, emission spectrum, CIEx, and CIEy at this time were measured with a luminance meter (a multi-channel spectrometer PMA12 available from Hamamatsu Photonics. Co., Ltd.). CIEx and CIEy respectively indicate the x value and y value of CIE chromaticity coordinates. In addition, the wavelength showing the maximum value in the electroluminescence (EL) intensity for the wavelength was defined as the emission peak wavelength (nm), and the wavelength width corresponding to a half of the emission peak wavelength (nm) was defined as the FWHM (nm).

### Examples 2 and 4 and Comparative Example 1

Organic light-emitting devices were manufactured in the same manner as in Example 1, except that compounds shown in Table 6 were used as a dopant material in formation of the emission layer. Evaluation was performed on the organic light-emitting devices.

In this evaluation, the emission peak wavelength may be about 450 nm or more and about 475 nm or less. In addition, the FWHM may be 30 nm or less. The CIEx was 0.160 or less, and the CIEy was 0.280 or less.

The evaluation results for the organic light-emitting devices of Examples 1 to 4 and Comparative Example 1 are shown in Table 6, and the emission spectrum is shown in FIG. 7.

TABLE 6

| | Emission layer | | | | | |
|---|---|---|---|---|---|---|
| | Host material | | | | Emission peak | Full width at |
| | composition | | | | wavelength | half maximum |
| | (mass ratio) | Dopant material | CIEₓ | CIE_{y} | (nm) | FWHM (nm) |
| Example 1 | H-H1:H-E1 = 6:4 | Compound 1-4 | 0.152 | 0.111 | 453.1 | 25.6 |
| Example 2 | H-H1:H-E1 = 6:4 | Compound 1-17 | 0.134 | 0.187 | 468.9 | 23.3 |
| Example 3 | H-H1:H-E1 = 6:4 | Compound 2-24 | 0.141 | 0.176 | 465.9 | 23.3 |
| Example 4 | H-H1:H-E1 = 6:4 | Compound 2-11 | 0.149 | 0.264 | 471.9 | 24.0 |
| Comparative | H-H1:H-E1 = 6:4 | Comparative | 0.281 | 0.439 | 523.0 | 135.7 |
| Example 1 | | Compound R2 | | | | |

In the organic light-emitting devices of Examples 1 to 4 using Compounds according to embodiments, the dopant emitted light. On the other hand, in Comparative Example 1 using Comparative Compound R2, emission of exciplex in addition to dopant was confirmed.

### Examples 5 to 8

Organic light-emitting devices were manufactured in the same manner as in Example 1, except that compounds shown in Table 7 were used as a dopant material in formation of the emission layer. Evaluation was performed on the organic light-emitting devices. In addition, the structure of Phosphorescent Complex Pt-1 is as follows.

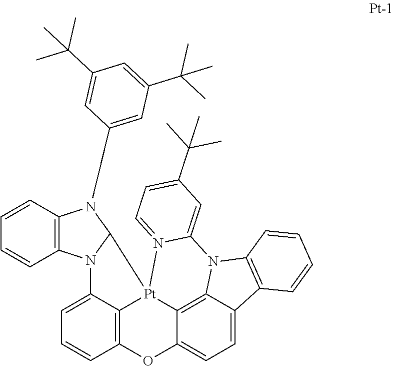

TABLE 7

| | Emission layer | | | | | |
|---|---|---|---|---|---|---|
| | Host material | | | | Emission peak | Full width at |
| | composition | | | | wavelength | half maximum |
| | (mass ratio) | Dopant material | CIEₓ | CIE_{y} | (nm) | FWHM (nm) |
| Example 5 | H-H1:H-E1 = 6:4 | Pt-1:Compound 1-4 = 32:1 | 0.168 | 0.221 | 462.1 | 24.1 |
| Example 6 | H-H1:H-E1 = 6:4 | Pt-1:Compound 1-17 = 32:1 | 0.164 | 0.218 | 465.1 | 23.3 |
| Example 7 | H-H1:H-E1 = 6:4 | Pt-1:Compound 2-24 = 32:1 | 0.167 | 0.227 | 462.9 | 22.6 |
| Example 8 | H-H1:H-E1 = 6:4 | Pt-1:Compound 2-11 = 32:1 | 0.168 | 0.242 | 465.9 | 25.5 |

The organic EL devices of Examples 5 to 8 using Compounds according to embodiments showed desirable peak wavelengths, FWHM, CIEx, and CIEy even when Phosphorescent Complex Pt-1 was combined with a dopant.

As apparent from the foregoing description, an organic light-emitting device including the condensed cyclic compound may have improved efficiency and/or colorimetric purity.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A condensed cyclic compound represented by Formula 1: 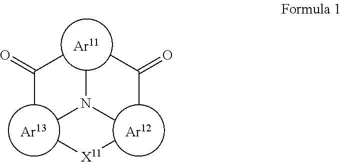
wherein, in Formula 1,
Ar¹¹, Ar¹², and Ar¹³ are each independently a substituted or unsubstituted group derived from an aromatic ring having 6 or more and 30 or less ring-forming atoms or a substituted or unsubstituted group derived from an aromatic ring having 5 or more and 30 or less ring-forming atoms,
X¹¹ is a single bond, —O—, —S—, —Se—, —NR^{X11}—, —CR^{X12}R^{X13}—, or —SiR^{X14}R^{X15}—,
when X¹¹ is —O—, —S—, or —CR^{X12}R^{X13}—, Ar¹¹ is a substituted group derived from an aromatic ring having 6 or more and 30 or less ring-forming atoms or a substituted or unsubstituted group derived from an aromatic ring having 5 or more and 30 or less ring-forming atoms,
R^{X11}, R^{X12}, R^{X13}, R^{X14}, and R^{X15} are each independently a hydrogen atom, a deuterium atom, a substituted or unsubstituted alkyl group, a substituted or unsubstituted alkenyl group, a substituted or unsubstituted alkynyl group, a substituted or unsubstituted aryl group, a substituted or unsubstituted arylalkyl group, or a substituted or unsubstituted heteroaryl group,
R^{X12} and R^{X13} are optionally bound to each other to form a ring, and R^{X14} and R^{X15} are optionally bound to each other to form a ring.

2. The condensed cyclic compound of claim 1, represented by Formula 1 and Formula 2: 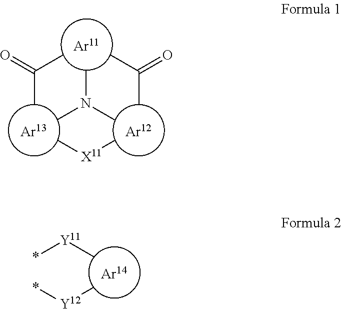
wherein, in Formulae 1 and 2,
Ar¹¹, Ar¹², Ar¹³, and Ar¹⁴ are each independently a substituted or unsubstituted group derived from an aromatic ring having 6 or more and 30 or less ring-forming atoms or a substituted or unsubstituted group derived from an aromatic ring having 5 or more and 30 or less ring-forming atoms,
X¹¹ is a single bond, —O—, —S—, —Se—, —NR^{X11}—, —CR^{X12}R^{X13}—, or —SiR^{X14}R^{X15}—,
R^{X11}, R^{X12}, R^{X13}, R^{X14}, and R^{X15} are each independently a hydrogen atom, a deuterium atom, a substituted or unsubstituted alkyl group, a substituted or unsubstituted alkenyl group, a substituted or unsubstituted alkynyl group, a substituted or unsubstituted aryl group, a substituted or unsubstituted arylalkyl group, or a substituted or unsubstituted heteroaryl group,
Y¹¹ and Y¹² are each a single bond, —O—, —S—, —Se—, —NR^{Y11}—, —CR^{Y12}R^{Y13}—, or —SiR^{Y14}R^{Y15}—,
R^{Y11}, R^{Y12}, R^{Y13}, R^{Y14}, and R^{Y15} are each independently a hydrogen atom, a deuterium atom, a substituted or unsubstituted alkyl group, a substituted or unsubstituted alkenyl group, a substituted or unsubstituted alkynyl group, a substituted or unsubstituted aryl group, a substituted or unsubstituted arylalkyl group, or a substituted or unsubstituted heteroaryl group,
at least one of Y¹¹ and Y¹² is a group other than a single bond, and
two * in Formula 2 are bound to a ring-forming atom of Ar¹¹ in Formula 1, a ring-forming atom of Ar¹², or a ring-forming atom of Ar¹³.

3. The condensed cyclic compound of claim 1 represented by Formula 3: 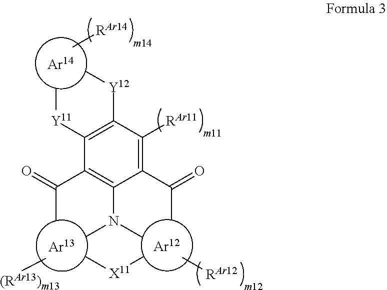
wherein, in Formula 3,
Ar¹², Ar¹³, and Ar¹⁴ are each independently a substituted or unsubstituted group derived from an aromatic ring having 6 or more and 30 or less ring-forming atoms or a substituted or unsubstituted group derived from an aromatic ring having 5 or more and 30 or less ring-forming atoms,
X¹¹ is a single bond, —O—, —S—, —Se—, —NR^{X11}—, —CR^{X12}R^{X13}—, or —SiR^{X14}R^{X15}—,
R^{X11}, R^{X12}, R^{X13}, R^{X14}, and R^{X15} are each independently a hydrogen atom, a deuterium atom, a substituted or unsubstituted alkyl group, a substituted or unsubstituted alkenyl group, a substituted or unsubstituted alkynyl group, a substituted or unsubstituted aryl group, a substituted or unsubstituted arylalkyl group, or a substituted or unsubstituted heteroaryl group,
Y¹¹ and Y¹² are each a single bond, —O—, —S—, —Se—, —NR^{Y11}—, —CR^{Y12}R^{Y13}—, or —SiR^{Y14}R^{Y15}—,
R^{Y11}, R^{Y12}, R^{Y13}, R^{Y14}, and R^{Y15} are each independently a hydrogen atom, a deuterium atom, a substituted or unsubstituted alkyl group, a substituted or unsubstituted alkenyl group, a substituted or unsubstituted alkynyl group, a substituted or unsubstituted aryl group, a substituted or unsubstituted arylalkyl group, or a substituted or unsubstituted heteroaryl group,
at least one of Y¹¹ and Y¹² is a group other than a single bond,
R^{Ar11}, R^{Ar12}, R^{Ar13}, and R^{Ar14} are each independently a deuterium atom, a halogen atom, a cyano group, an alkyl group, a haloalkyl group, an alkenyl group, an alkynyl group, —SiR¹¹R¹²R¹³, —NR¹⁴R¹⁵, an aryl group, an alkylaryl group, a heteroaryl group, an alkylheteroaryl group, an arylalkyl group, an alkoxy group, an aryloxy group, an alkylaryloxy group, a heteroaryloxy group, an alkylheteroaryloxy group, an alkylthio group, an arylthio group, an alkylarylthio group, a heteroarylthio group, or an alkylheteroarylthio group,
R¹¹, R¹², R¹³, R¹⁴, and R¹⁵ are each independently a hydrogen atom, a deuterium atom, an alkyl group, an alkenyl group, an alkynyl group, an aryl group, an arylalkyl group, or a heteroaryl group,
m11 is 0 or 1, and
m127 m137 and m14 are each independently 0, 1, 2, 3, or 4.

4. The condensed cyclic compound of claim 3, wherein m12, m13, and m14 are each independently 0 or 1.

5. The condensed cyclic compound of claim 1 represented by any one of Formulae 1-1 to 1-18:

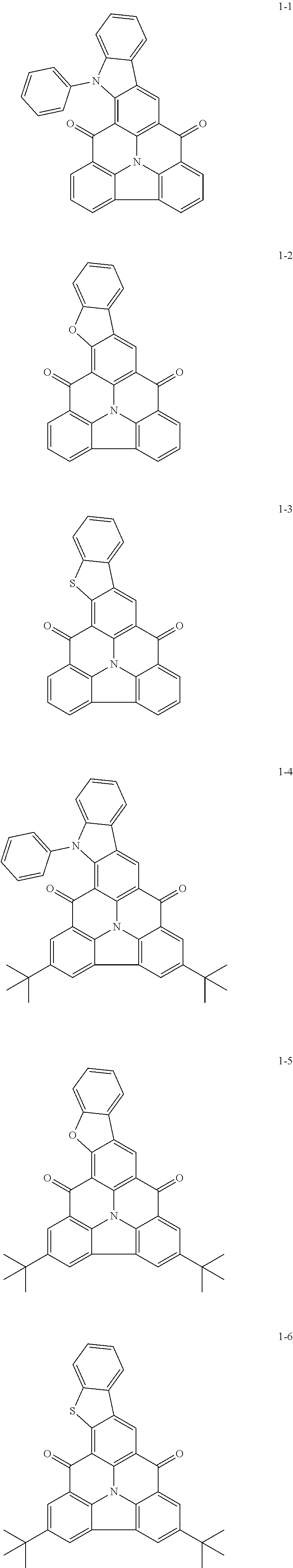

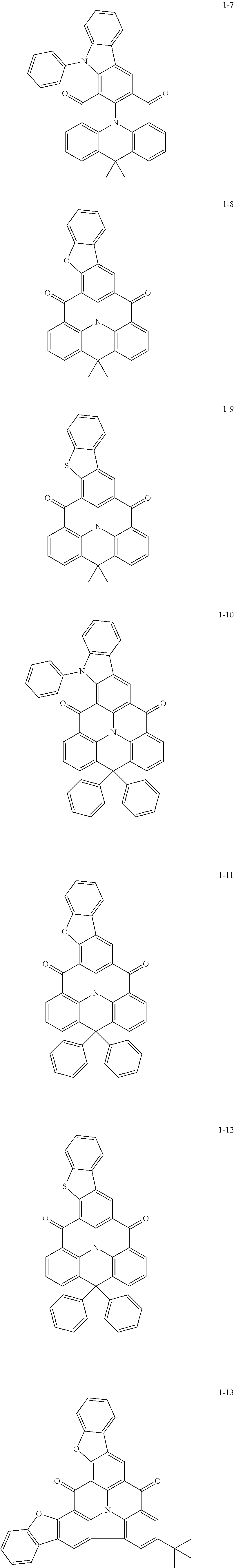

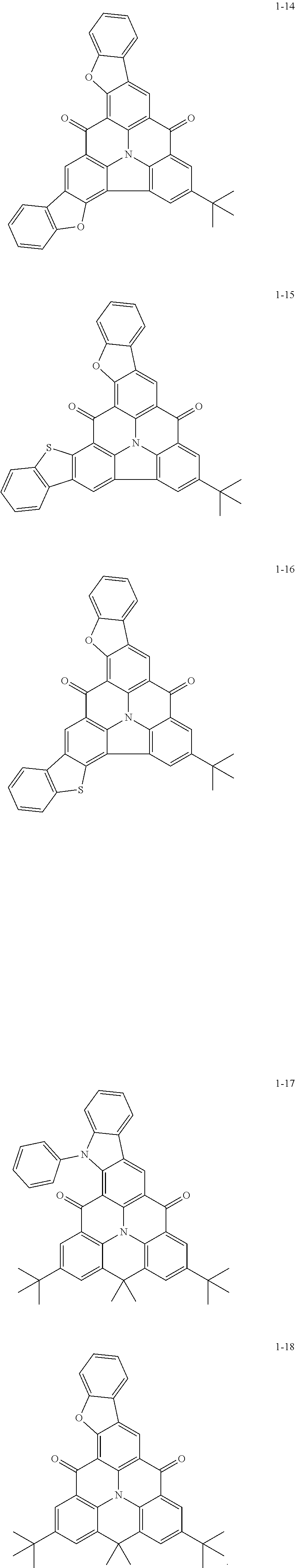

6. The condensed cyclic compound of claim 1 represented by Formula 4: 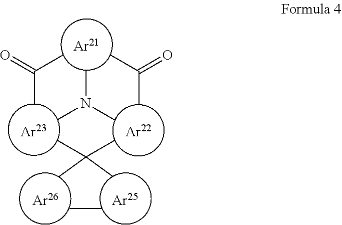
wherein, in Formula 4,
Ar²¹ is (a) a substituted group derived from an aromatic ring having 6 or more and 30 or less ring-forming atoms or (b) a substituted or unsubstituted group derived from a heteroaromatic ring having 5 or more and 30 or less ring-forming atoms,
wherein (i) at least one hydrogen atom of (a) a group derived from an aromatic ring or (b) a group derived from a heteroaromatic ring is substituted with an alkyl group, a haloalkyl group, —SiR²¹R²²R²³, —NR²⁴R²⁵, an alkylaryl group, an alkylheteroaryl group, an alkoxy group, an alkylaryloxy group, an alkylheteroaryloxy group, an alkylthio group, an alkylarylthio group, or an alkylheteroarylthio group (where R²¹, R²², R²³, R²⁴, and R²⁵ are each independently a hydrogen atom, a deuterium atom, an alkyl group, an alkenyl group, an alkynyl group, an aryl group, an arylalkyl group, or a heteroaryl group) is bound to two * in Formula 5, or (ii) a ring-forming atom of (a) a group derived from an aromatic ring or (b) a group derived from a heteroaromatic ring is bound to two * in Formula 5,
Ar²², Ar²³, Ar²⁵, and Ar²⁶ are each independently a substituted or unsubstituted group derived from an aromatic ring having 6 or more and 30 or less ring-forming atoms or a substituted or unsubstituted group derived from an aromatic ring having 5 or more and 30 or less ring-forming atoms, 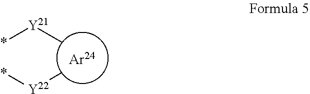
wherein, in Formula 5,
Ar²⁴ is a substituted or unsubstituted group derived from an aromatic ring having 6 or more and 30 or less ring-forming atoms or a substituted or unsubstituted group derived from an aromatic ring having 5 or more and 30 or less ring-forming atoms,
Y²¹ and Y²² are each a single bond, —O—, —S—, —Se—, —NR^{Y21}—, —CR^{Y22}R^{Y23}—, or —SiR^{Y24}R^{Y25}—,
R^{Y21}, R^{Y22}, R^{Y23}, R^{Y24}, and R^{Y25} are each independently a hydrogen atom, a deuterium atom, a substituted or unsubstituted alkyl group, a substituted or unsubstituted alkenyl group, a substituted or unsubstituted alkynyl group, a substituted or unsubstituted aryl group, a substituted or unsubstituted arylalkyl group, or a substituted or unsubstituted heteroaryl group, and
at least one of Y²¹ and Y²² is a group other than a single bond.

7. The condensed cyclic compound of claim 1, represented by Formula 6: 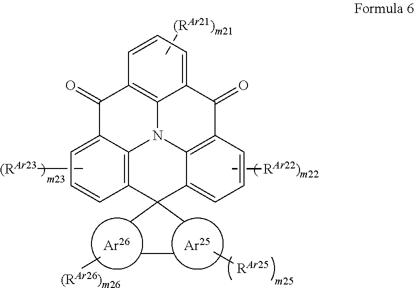
wherein, in Formula 6,
Ar²⁵ and Ar²⁶ are each independently a substituted or unsubstituted group derived from an aromatic ring having 6 or more and 30 or less ring-forming atoms or a substituted or unsubstituted group derived from an aromatic ring having 5 or more and 30 or less ring-forming atoms,
R^{Ar21} is an alkyl group, a haloalkyl group, —SiR²¹R²²R²³, —NR²⁴R²⁵, an alkylaryl group, an alkylheteroaryl group, an alkoxy group, an alkylaryloxy group, an alkylheteroaryloxy group, an alkylthio group, an alkylarylthio group, or an alkylheteroarylthio group,
R^{Ar22}, R^{Ar23}, R^{Ar25}, and R^{Ar26} are each independently a deuterium atom, a halogen atom, a cyano group, an alkyl group, a haloalkyl group, an alkenyl group, an alkynyl group, —SiR²¹R²²R²³, —NR²⁴R²⁵, an aryl group, an alkylaryl group, a heteroaryl group, an alkylheteroaryl group, an arylalkyl group, an alkoxy group, an aryloxy group, an alkylaryloxy group, a heteroaryloxy group, an alkylheteroaryloxy group, an alkylthio group, an arylthio group, an alkylarylthio group, a heteroarylthio group, or an alkylheteroarylthio group,
R²¹, R²², R²³, R²⁴, and R²⁵ are each independently a hydrogen atom, a deuterium atom, an alkyl group, an alkenyl group, an alkynyl group, an aryl group, an arylalkyl group, or a heteroaryl group,
m21 is 1, 2, or 3,
m22 and m23 are each independently 0, 1, 2, or 3, and
m25 and m26 are each independently 0, 1, 2, 3, or 4.

8. The condensed cyclic compound of claim 7, wherein m22, m23, m25, and m26 are each independently 0 or 1.

9. The condensed cyclic compound of claim 1, represented by Formula 7: 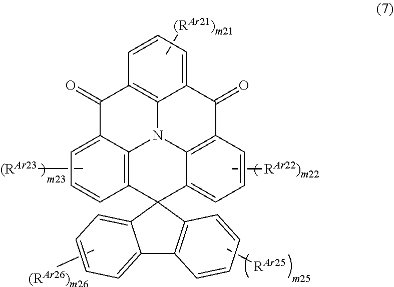
wherein, in Formula 7,
R^{Ar21} is an alkyl group, a haloalkyl group, —SiR²¹R²²R²³, —NR²⁴R²⁵, an alkylaryl group, an alkylheteroaryl group, an alkoxy group, an alkylaryloxy group, an alkylheteroaryloxy group, an alkylthio group, an alkylarylthio group, or an alkylheteroarylthio group,
R^{Ar22}, R^{Ar23}, R^{Ar25}, and R^{Ar26} are each independently a deuterium atom, a halogen atom, a cyano group, an alkyl group, a haloalkyl group, an alkenyl group, an alkynyl group, —SiR²¹R²²R²³, —NR²⁴R²⁵, an aryl group, an alkylaryl group, a heteroaryl group, an alkylheteroaryl group, an arylalkyl group, an alkoxy group, an aryloxy group, an alkylaryloxy group, a heteroaryloxy group, an alkylheteroaryloxy group, an alkylthio group, an arylthio group, an alkylarylthio group, a heteroarylthio group, or an alkylheteroarylthio group,
R²¹, R²², R²³, R²⁴, and R²⁵ are each independently a hydrogen atom, a deuterium atom, an alkyl group, an alkenyl group, an alkynyl group, an aryl group, an arylalkyl group, or a heteroaryl group,
m21 is 1, 2, or 3,
m22 and m23 are each independently 0, 1, 2, or 3, and
m25 and m26 are each independently 0, 1, 2, 3, or 4.

10. The condensed cyclic compound of claim 1, represented by any one of Formulae 2-1 to 2-29:

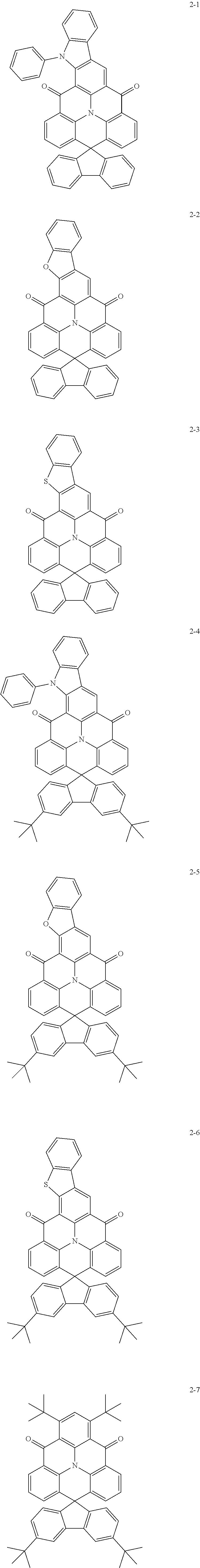

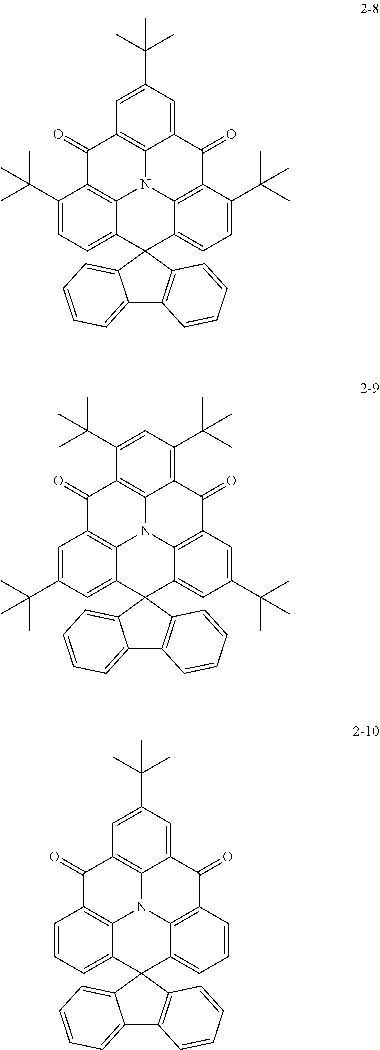

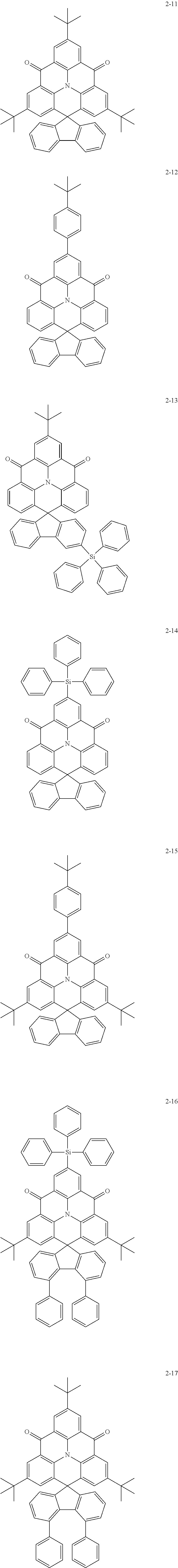

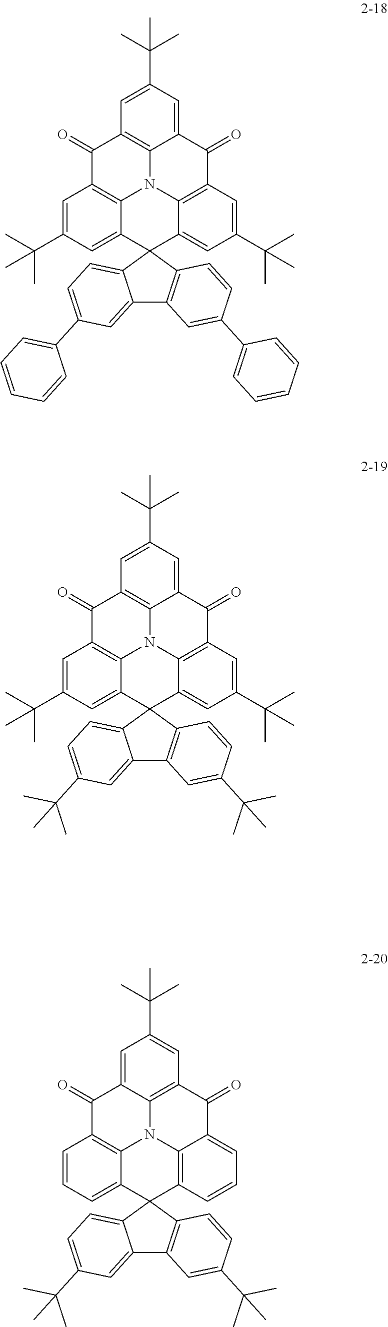

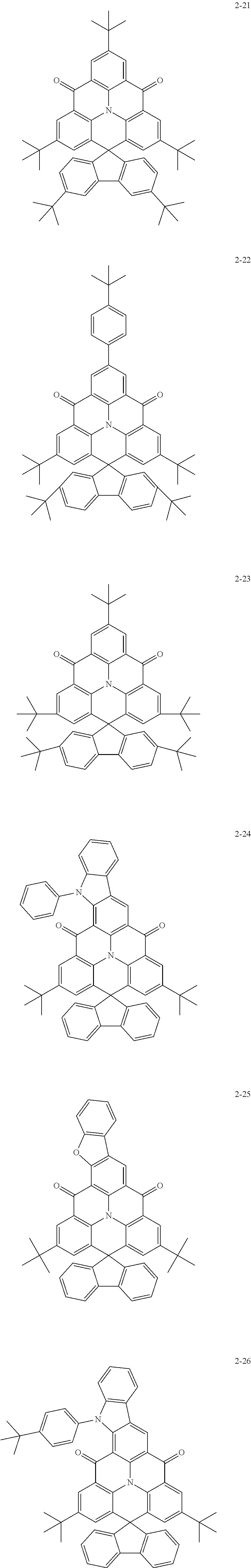

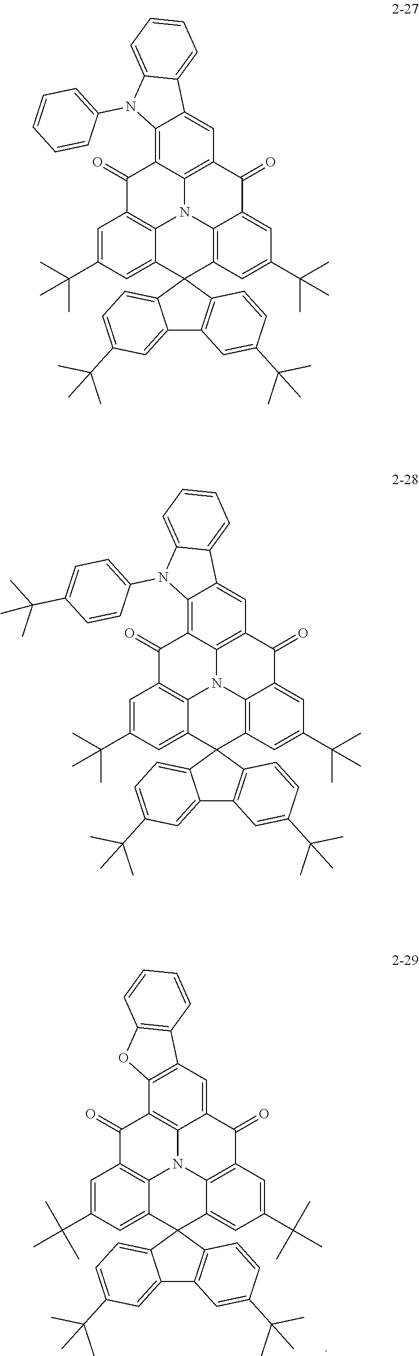

11. The condensed cyclic compound of claim 1, wherein
an energy level of a highest occupied molecular orbital (HOMO) of the condensed cyclic compound is about −5.80 eV or more and about −4.40 eV or less, and
an energy level of a lowest unoccupied molecular orbital (LUMO) of the condensed cyclic compound is about −2.40 eV or more and about −0.80 eV or less.

12. The condensed cyclic compound of claim 1, wherein
a peak of a fluorescence wavelength of the condensed cyclic compound is about 360 nanometers (nm) or more and about 515 nm or less, and
a half-width of a peak in a fluorescence emission spectrum of the condensed cyclic compound is 30 nm or less.

13. The condensed cyclic compound of claim 1, wherein a rearrangement energy of the condensed cyclic compound is about 0 eV or more and 0.110 eV or less.

14. An organic light-emitting device comprising: a first electrode; a second electrode; an organic layer between the first electrode and the second electrode and comprising an emission layer; and the condensed cyclic compound of claim 1.

15. The organic light-emitting device of claim 14, wherein the emission layer comprises the condensed cyclic compound.

16. The organic light-emitting device of claim 14, wherein
the emission layer further comprises a host,
the host and the condensed cyclic compound are different from each other, and
the emission layer consists of the host and the condensed cyclic compound.

17. The organic light-emitting device of claim 16, wherein the host does not emit light, and the condensed cyclic compound emits light.

18. The organic light-emitting device of claim 14, wherein
the emission layer further comprises a host and a dopant,
the host, the dopant, and the condensed cyclic compound are different from each other, and
the emission layer consists of the host, the dopant, and the condensed cyclic compound.

19. The organic light-emitting device of claim 18, wherein the host and the condensed cyclic compound each do not emit light, and the dopant emits light.

20. An electronic apparatus comprising the organic light-emitting device of claim 14.
